SEQUENCE LISTING (50 sequences)

SEQ ID NO: 1 - protein, 121 aa, synthetic construct
QSVEESGGRLVTPGTPLTLTCTVSGFSLNTYAMTWVRQAPGKGLEWIGIIVASGGTYYASWAKGRFTISRTSTTVVMKITSPTTEDTATYFCARGGMGSDWGESLDAFDPWGQGTLVTVSS

SEQ ID NO: 2 - protein, 110 aa, synthetic construct
DIVMTQTPASVEAAVGGTVTINCQASGNIYSFLAWYQQKPGQPPKLLIYTASNLETGVPSRFSGSGSGTEYTLTISGVQCDDAATYYCLGGLYSSDVSYAFGGGTEVVVK

SEQ ID NO: 3 - protein, 448 aa, synthetic construct
QSVEESGGRLVTPGTPLTLTCTVSGFSLNTYAMTWVRQAPGKGLEWIGIIVASGGTYYASWAKGRFTISRTSTTVVMKITSPTTEDTATYFCARGGMGSDWGESLDAFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 451 aa, synthetic construct
QSVEESGGRLVTPGTPLTLTCTVSGFSLNTYAMTWVRQAPGKGLEWIGIIVASGGTYYASWAKGRFTISRTSTTVVMKITSPTTEDTATYFCARGGMGSDWGESLDAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 217 aa, synthetic construct
DIVMTQTPASVEAAVGGTVTINCQASGNIYSFLAWYQQKPGQPPKLLIYTASNLETGVPSRFSGSGSGTEYTLTISGVQCDDAATYYCLGGLYSSDVSYAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 124 aa, synthetic construct
QVQLVESGGGVVQPGTSLRLSCAASGFSLNTYAMTWVRQAPGKGLEWVAIIVASGGTYYASWAKGRFTISRDTSKNTVVMQITSLRAEDTAVYFCARGGMGSDWGESLDAFDPWGQGTLVTVSS

SEQ ID NO: 7 - protein, 124 aa, synthetic construct
EVQLLESGGGLVQPGGSLRLSCAASGFSLNTYAMTWVRQAPGKGLEWVSIIVASGGTYYASWAKGRFTISRDNSKNTLYLQMNSLTVEDTAVYFCARGGMGSDWGESLDAFDPWGQGTLVTVSS

SEQ ID NO: 8 - protein, 124 aa, synthetic construct
EVQLVESGGDLAQPGGSLRLSCAVSGFSLNTYAMTWVRQAPGKGLEWVSIIVASGGTYYASWAKGRFTISRDNSKNTVYLQMTSLRAEDTALYFCARGGMGSDWGESLDAFDPWGQGTLVTVSS

SEQ ID NO: 9 - protein, 124 aa, synthetic construct
EVQLVESGGGLVQPGGSLRLSCAASGFSLNTYAMTWVRQAPGKGLEWISIIVASGGTYYASWAKGRFTISRDNAKSSVYLQLTSLTTEDTAVYYCARGGMGSDWGESLDAFDPWGQGTMVTVSS

SEQ ID NO: 10 - protein, 124 aa, synthetic construct
QVQLEESGGRLVQPGTSLRLSCAVSGFSLNTYAMTWVRQAPGKGLEWIGIIVASGGTYYASWAKGRFTISRDTSKNTLYLQITSLRAEDTATYFCARGGMGSDWGESLDAFDPWGQGTLVTVSS

SEQ ID NO: 11 - protein, 110 aa, synthetic construct
DIQMTQSPSTLSASVGDRVTITCQASGNIYSFLAWYQQKPGKAPKLLIYTASNLETGVPSRFSGSGSGTEYTLTISSLQPDDFATYYCLGGLYSSDVSYAFGPGTKVEIK

SEQ ID NO: 12 - protein, 110 aa, synthetic construct
DIQMTQSPSSLSASVGDRVTITCQASGNIYSFLAWYQQKPGKVPKLLIYTASNLETGVPSRFSGSGSGTDYTLTISSLQSEDAATYYCLGGLYSSDVSYAFGGGTKVVIK

SEQ ID NO: 13 - protein, 110 aa, synthetic construct
DIVMTQSPSSVSASVGDRVTITCQASGNIYSFLAWYQQKPGKAPKLLIYTASNLETGVPSRFSGSGSGTDFTLTISSLQPEDFATYYCLGGLYSSDVSYAFGGGTKVVIK

SEQ ID NO: 14 - protein, 110 aa, synthetic construct
DVVMTQSPSTVSASVGDRVTLTCQASGNIYSFLAWYQQKPGQPPKLLIYTASNLETGVPDRFSGSGSGTDYTLTISSLQADDFATYYCLGGLYSSDVSYAFGQGTKVEIK

SEQ ID NO: 15 - protein, 110 aa, synthetic construct
DIQMTQTPSTVEASVGGTVTITCQASGNIYSFLAWYQQKPGQPPKLLIYTASNLETGVPSRFSGSGSGTEYTLTISGLQSDDAATYYCLGGLYSSDVSYAFGGGTEVVVK

SEQ ID NO: 16 - protein, 8 aa, synthetic construct
GFSLNTYA

SEQ ID NO: 17 - protein, 7 aa, synthetic construct
IVASGGT

SEQ ID NO: 18 - protein, 18 aa, synthetic construct
ARGGMGSDWGESLDAFDP

SEQ ID NO: 19 - protein, 6 aa, synthetic construct
GNIYSF

SEQ ID NO: 20 - skipped

SEQ ID NO: 21 - protein, 12 aa, synthetic construct
LGGLYSSDVSYA

SEQ ID NO: 22 - protein, 5 aa, synthetic construct
TYAMT

SEQ ID NO: 23 - protein, 16 aa, synthetic construct
IIVASGGTYYASWAKG

SEQ ID NO: 24 - protein, 16 aa, synthetic construct
GGMGSDWGESLDAFDP

SEQ ID NO: 25 - protein, 11 aa, synthetic construct
QASGNIYSFLA

SEQ ID NO: 26 - protein, 7 aa, synthetic construct
TASNLET

SEQ ID NO: 27 - protein, 12 aa, synthetic construct
LGGLYSSDVSYA

SEQ ID NO: 28 - protein, 451 aa, synthetic construct
QVQLVESGGGVVQPGTSLRLSCAASGFSLNTYAMTWVRQAPGKGLEWVAIIVASGGTYYASWAKGRFTISRDTSKNTVVMQITSLRAEDTAVYFCARGGMGSDWGESLDAFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 451 aa, synthetic construct
EVQLLESGGGLVQPGGSLRLSCAASGFSLNTYAMTWVRQAPGKGLEWVSIIVASGGTYYASWAKGRFTISRDNSKNTLYLQMNSLTVEDTAVYFCARGGMGSDWGESLDAFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 451 aa, synthetic construct
EVQLVESGGDLAQPGGSLRLSCAVSGFSLNTYAMTWVRQAPGKGLEWVSIIVASGGTYYASWAKGRFTISRDNSKNTVYLQMTSLRAEDTALYFCARGGMGSDWGESLDAFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 451 aa, synthetic construct
EVQLVESGGGLVQPGGSLRLSCAASGFSLNTYAMTWVRQAPGKGLEWISIIVASGGTYYASWAKGRFTISRDNAKSSVYLQLTSLTTEDTAVYYCARGGMGSDWGESLDAFDPWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 451 aa, synthetic construct
QVQLEESGGRLVQPGTSLRLSCAVSGFSLNTYAMTWVRQAPGKGLEWIGIIVASGGTYYASWAKGRFTISRDTSKNTLYLQITSLRAEDTATYFCARGGMGSDWGESLDAFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 454 aa, synthetic construct
QVQLVESGGGVVQPGTSLRLSCAASGFSLNTYAMTWVRQAPGKGLEWVAIIVASGGTYYASWAKGRFTISRDTSKNTVVMQITSLRAEDTAVYFCARGGMGSDWGESLDAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 454 aa, synthetic construct
EVQLLESGGGLVQPGGSLRLSCAASGFSLNTYAMTWVRQAPGKGLEWVSIIVASGGTYYASWAKGRFTISRDNSKNTLYLQMNSLTVEDTAVYFCARGGMGSDWGESLDAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 454 aa, synthetic construct
EVQLVESGGDLAQPGGSLRLSCAVSGFSLNTYAMTWVRQAPGKGLEWVSIIVASGGTYYASWAKGRFTISRDNSKNTVYLQMTSLRAEDTALYFCARGGMGSDWGESLDAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 454 aa, synthetic construct
EVQLVESGGGLVQPGGSLRLSCAASGFSLNTYAMTWVRQAPGKGLEWISIIVASGGTYYASWAKGRFTISRDNAKSSVYLQLTSLTTEDTAVYYCARGGMGSDWGESLDAFDPWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 454 aa, synthetic construct
QVQLEESGGRLVQPGTSLRLSCAVSGFSLNTYAMTWVRQAPGKGLEWIGIIVASGGTYYASWAKGRFTISRDTSKNTLYLQITSLRAEDTATYFCARGGMGSDWGESLDAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 217 aa, synthetic construct
DIQMTQSPSTLSASVGDRVTITCQASGNIYSFLAWYQQKPGKAPKLLIYTASNLETGVPSRFSGSGSGTEYTLTISSLQPDDFATYYCLGGLYSSDVSYAFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 217 aa, synthetic construct
DIQMTQSPSSLSASVGDRVTITCQASGNIYSFLAWYQQKPGKVPKLLIYTASNLETGVPSRFSGSGSGTDYTLTISSLQSEDAATYYCLGGLYSSDVSYAFGGGTKVVIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 217 aa, synthetic construct
DIVMTQSPSSVSASVGDRVTITCQASGNIYSFLAWYQQKPGKAPKLLIYTASNLETGVPSRFSGSGSGTDFTLTISSLQPEDFATYYCLGGLYSSDVSYAFGGGTKVVIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 217 aa, synthetic construct
DVVMTQSPSTVSASVGDRVTLTCQASGNIYSFLAWYQQKPGQPPKLLIYTASNLETGVPDRFSGSGSGTDYTLTISSLQADDFATYYCLGGLYSSDVSYAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 217 aa, synthetic construct
DIQMTQTPSTVEASVGGTVTITCQASGNIYSFLAWYQQKPGQPPKLLIYTASNLETGVPSRFSGSGSGTEYTLTISGLQSDDAATYYCLGGLYSSDVSYAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 448 aa, synthetic construct
QVQLKESGPGLVAPSQSLSITCTVSGFSLTNYGVSWVRQPPGKGLEWLGIIWGDGSTNYHSALISRLSISKDNSKSQVFLKLNSLQADDTATYYCAKPNWDTYAMDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 214 aa, synthetic construct
DIQMTQTTSSLSASLGDRVTISCRASQDISNFLNWYQQKPDGTVKLLIYCTSKLHSGVPSRFSGSGSETDYSLTISNLEQEDIATYFCQQGNTLPPTFGGGTKLEIIRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 450 aa, synthetic construct
QIQLVQSGPELKKPGETVKISCKASGYTFTNYGMNWVKQAPGKGLKWMGWINTYIGEPIYADDFKGRFAFSLETSASTAYLQINNLKNEDMATYFCARRSDYDGYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 219 aa, synthetic construct
DIVMSQSPSSLAVSVGERVTMSCKSSQSLLYSGNQKNYLAWYQQKPGQSPKLLIYWASTRESGVPDRFTGSGSGTDFTLTISSVKAEDLAVYYCQQHDSYPTFGGGSRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 455 aa, synthetic construct
QVQLVQSGAEVKKPGASVKVSCKASGYTFTDYYINWVRQAPGQGLEWMGNVNPNDGGTTYNQKFKGRVTMTTDTSTSTAYMELRSLRSDDTAVYYCARREIYFYGTIYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 218 aa, synthetic construct
DIQLTQSPSFLSASVGDRVTITCRASESVDYYGNSFMYWYQQKPGKAPKLLIYFASNLESGVPSRFSGSGSGTEFTLTISSLQPEDFATYYCQQNNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 448 aa, synthetic construct
EVQLQQWGAGLLKPSETLSLTCAVYGGSFSGYYWSWIRQPPGKGLEWIGEINHAGSTNYNPSLKSRVTISVDTSKNQFSLKLSSVTAADTAVYYCARLPTRWVTTRYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 217 aa, synthetic construct
ESVLTQPPSVSGAPGQRVTISCTGSSSNIGAGYDVHWYQQLPGTAPKLLIYGDSNRPSGVPDRFSVSKSGASASLAITGLQAEDEADYYCQSFDNSLSAYVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2025/0002577 A1
(43) Pub. Date: Jan. 2, 2025

(54) LILRB2/1 ANTIBODY PRODUCTS AND METHODS

(71) Applicant: ONCORESPONSE, INC., Seattle, WA (US)

(72) Inventors: Meghan Zuck, Seattle, WA (US); Peter Probst, Seattle, WA (US); Thomas J. Graddis, Seattle, WA (US); Myriam N. Bouchlaka Bay, Seattle, WA (US); Kamal D. Puri, Seattle, WA (US)

(21) Appl. No.: 18/634,613

(22) Filed: Apr. 12, 2024

Related U.S. Application Data
(60) Provisional application No. 63/527,282, filed on Jul. 17, 2023.
(60) Provisional application No. 63/459,096, filed on Apr. 13, 2023.

(51) Int. Cl. C07K 16/28; A61K 47/68; A61P 35/00; G01N 33/68

(52) U.S. Cl. CPC C07K 16/2803; A61K 47/6849; A61P 35/00; G01N 33/6854; C07K 2317/31; G01N 2333/70503

(57) ABSTRACT

The disclosure relates to LILRB2/1 antibody products and methods of use thereof. The antibody products specifically bind to LILRB1 and LILRB2 on cells such as myeloid cells, lymphocytes, or cancer cells. The antibody products can be used in methods of treatment of disease, such as methods cancer immunotherapy.

### CROSS-REFERENCE TO RELATED APPLICATIONS

This application claims priority to U.S. Provisional Application No. 63/459,096, filed Apr. 13, 2023 and U.S. Provisional Application No. 63/527,282, filed Jul. 17, 2023, both of which are incorporated herein by reference in their entirety.

### INCORPORATION BY REFERENCE OF THE SEQUENCE LISTING

This application contains, as a separate part of disclosure, a Sequence Listing in computer-readable form (Filename: 59003_SeqListing.xml; 54,121 bytes—XML file created Apr. 8, 2024) which is incorporated by reference herein in its entirety.

### FIELD

The disclosure relates to LILRB2/1 antibody products and related methods. The products disclosed herein bind to certain LILRB family members expressed on human cells such as macrophages or cancer cells. The products can be used in methods of treatment of disease, such as in methods of treating cancer.

### BACKGROUND

Checkpoint inhibitor (CPI) therapy targets immune checkpoints to manipulate and/or modulate the immune system, including both innate immune responses and adaptive immune responses. The recent development of checkpoint inhibitor antibodies has transformed the way malignant solid tumors are treated. Despite the success of immunotherapy, CPIs have failed to improve the outcomes of those with tumors that are not infiltrated by immune cells, so-called ‘cold’ tumors. One cause for the failure of checkpoint inhibitors in cold tumors is the immunosuppressive nature of the tumor microenvironment (TME) [Heinhuis et al., JAMA OncoL 2020 6(1):100-7]. Immunosuppressive myeloid cells such as myeloid derived suppressor cells (MDSC) and tumor-associated macrophages (TAM) are important components of the TME and contribute to immune evasion by many solid tumors [Fleming et al., Front Immunol. 2018 9:398 (doi: 10.3389/fimmu.2018.00398); Garvin et al., J Cancer Res Clin Oncol. 2018 144(7):1253-63]. High levels of tumor infiltration by MDSCs and TAMs generally predict an unfavorable prognosis in solid tumor subjects [Ai et al., BMC Cancer. 2018 18(1):1220; Chen et al., J Biomed Sci. 2019 26(1):78].

The LILR family includes six stimulatory members and four inhibitory members, in two broad subfamilies, LILRA and LILRB, respectively. The LILRB subfamily members contain two or four extracellular immunoglobulin domains, a transmembrane domain, and two to four cytoplasmic immunoreceptor tyrosine-based inhibitory motifs (ITIMs). LILR can exert inhibitory or stimulatory immunomodulatory effects on a wide range of immune cells. See e.g., Lewis Marffy, Front Immunol. 2020 11:857; De Louche and Roghanian, JCI Insight 2022 7(2):e151553, doi:10.1172/jci.insight.151553.

Leukocyte immunoglobulin-like receptor B1 (LILRB1, also known as ILT2, LIR1, PIRB, MIR7, CD85j) and Leukocyte immunoglobulin-like receptor B2 (LILRB2, also known as ILT4, LIR2, MIR10, CD85d) are ITIM-containing immunosuppressive cell surface proteins expressed on immune cells and are known to inhibit the immune response. This inhibitory function is thought to control inflammatory responses and cytotoxicity, possibly contributing to limiting autoreactivity by the immune system. LILRB2 is primarily expressed on cells of the myeloid lineage (monocytes, macrophages, dendritic cells, and granulocytes) and LILRB1 is more broadly expressed, with expression on myeloid as well as lymphoid cells, including B cells and subsets of CD8 T cells and NK cells. In the TME, LILRB1 and LILRB2 are found on myeloid-derived suppressor cells (MDSCs) and tumor-supporting tumor-associated macrophages (TAMs) [Chen et al., J Clin Invest 2018 128(12):5647-62]. The presence of LILRB1 on additional immune cell types may impart greater functional differences between the two receptors, as LILRB1 expression is increased on NK cells of cancer patients and has been shown to correlate with resistance to NK cytotoxicity [Zhang, Mol Med Rep. 2012 5(4):910-6; Roberti et al., Eur J Immunol. 2015 45:1560-9; Chen et al. J Immunother Cancer. 2020 8(2):e000515]. NK cells play an important role in cancer immune responses through ADCC-mediated killing of cancer cells. Blockade of LILRB1 may contribute to lifting tumor-elicited immunosuppression of NK cell activity to potentiate the antitumor response.

Ligands of LILRB2 include, among others, HLA-A, -B, -C, -F, and -G, HLA-B27, CD1d and CD1c, and angiopoietin-like proteins 2 and 5. Ligands of LILRB1 include, among others, HLAA, B, C, F, and G, and HLA-B27 [Burshtyn and Morcos, J Immunol. 2016 196(3):947-55]. Trans or cis interactions of LILRB1 or LILRB2 with its ligands mediate immune suppression by myeloid cells and promote tumor immune evasion in the TME. The role of LILRB1 and LILRB2 interactions with nonclassical HLA-G has been studied extensively, as altered HLA-G expression has been reported within a tumor context and may be a marker of poor prognosis in cancer [Carosella et al., Trends Cancer. 2021 7(5):389-92; Chen et al., Int Immunopharmacol. 2022 109:108798; Lin and Yan, Mol Med. 2015 21(1):782-91]. See also PCT Pub. No. WO 2009/100135 A2.

LILRB1 is a type I transmembrane glycoprotein with four extracellular immunoglobulin-like (Ig-like) domains and four immunoreceptor tyrosine-based inhibitory motifs (ITIMs) within the cytoplasmic region for signal transduction [Cosman et al., Immunity. 1997 7:273-82]. LILRB1 is polymorphic, with a variety of transcript variants expressed within the human population [Liu et al., Immunogenetics. 2022 74(6):513-25]. The two membrane distal Ig domains (D1-D2) are responsible for interacting with HLA class I, with the D1 domain engaging with the α3 region of the HLA class I heavy chain, and the D2 domain interacting with beta 2 microglobulin (β2m) [Willcox et al., Nat Immunol. 2003 4(9):913-9.]. Since the α3 region of HLA class I antigens and β2m are relatively conserved, LILRB1 binds to a broad range of HLA class I antigens [Willcox et al., 2003. supra; Jones et al., J Immunol. 2011 186(5):2990-7; Liu et al., 2022, supra]. LILRB1 allotypes with amino acid changes in the D1-D2 region can be group into 9 variants, with variants 1, 2 and 3 representing the most common variants and the 9 variants showing frequency distribution in the populations [Liu et al., 2022, supra]. The effect of amino acid changes in the D1-D2 domains of LILRB1 on binding to select HLA class I antigens has been investigated [Yu et al., J Clin Invest 2018 128(4):1523-37, Kuroki et al., Hum Mol Genet 2005 14(16):2469-80]. The four most-studied amino acid changes in this region are L68P, A93T, I142T, and S155I, with the numbers corresponding to the full-length LILRB1 protein sequence. LILRB1 D1-D2 variants bind HLA Class I antigens with different avidities [Liu et al., 2022, supra]. These differences in binding avidities may be related to the diversity of immune responses among individuals, but how these polymorphisms affect LILRB1 activity and functionality are currently not well understood [Liu 2022, supra; Wang et al., Cell Mol Immunol. 2020 17:966-75 (2020)].

The combination of an anti-LILRB2 antibody with anti PD-1 treatment (TGI) in a humanized tumor model and with the combination of a LILRB1 blocking antibody with anti-PD-1 or anti-EGFR antibodies has been studied (Chen 2018, Mandel 2022). The effect of dual blockade of LILRB1 and LILRB2 has also been studied in tumor cells in vitro and [Hu et al., Cancer Res. 2022 82(12_Suppl):3433]. Antibodies that target and antagonize LILRB2 are currently being evaluated in clinical trials for the treatment of cancer, for example, MK-4830 (IgG4) (Agenus and Merck) and JTX-8064 (IgG4) (Jounce Therapeutics). See also Papadopoulos et al. ASCO abstract 2021 J Clin Oncol 39(15). Immune-Onc PCT Pub. Nos. WO 2022/087188 A1, Five Prime WO 2020/014132 A2, Mt. Sinai WO 2020_061059 A1, Jounce WO 2019/126514 A2, and Jounce WO 2016/111947 A2 disclose LILRB2 antibodies.

Merck PCT Pub. No. WO 2021/138079 A1 discloses use of a combination of a PD-1 antagonist, an ILT4 (LILRB2) antagonist, and lenvatinib (a kinase inhibitor) for the treatment of cancers. A LILRB2/PD-1 bispecific antibody, CTX-585 (Celldex Therapeutics), appears to be in preclinical development in the context of solid tumors. See also, PCT Pub. No. WO 2022/217019 A1.

Antibodies that target and antagonize both LILRB2 and LILRB1 are also in development, including NGM707 (NGM Biopharmaceuticals). See PCT Pub. No. WO 2021/222544 A1. PCT Pub. Nos. WO 2022/034524 A2 (Biond), WO 2022/026360 A2 (Univ. Texas), and LG Chemical WO 2022/025585 A1 disclose LILRB1 antibodies.

There remains a need in the art for LILRB antibody products and methods for their therapeutic use.

### SUMMARY

The present disclosure contemplates that the distinct pattern of expression and function of LILRB1 and LILRB2 allows complementary targeting approaches for cancer immunotherapy. Dual blockade of LILRB1 and LILRB2 receptors by a single antibody that restores both innate and adaptive immune responses is contemplated herein to enhance efficacy of checkpoint inhibitors. The disclosure provides LILRB2/1 antibody products (also sometimes referred to herein as “anti-LILRB2/1 antibody products”) and compositions comprising such antibody products, as well methods of their manufacture and methods for their use in the treatment of disease, including cancer. Treatment modalities blocking LILRB1 and LILRB2 binding to HLA ligands in the TME are provided to enhance efficacy of T cell checkpoint inhibitors.

A LILRB2/1 antibody product provided herein can be an intact antibody or can comprise an immunologically functional antibody fragment and thus LILRB2/1 antibody products include antibodies with a naturally occurring structure or recombinant structure, as well as other polypeptides, such as antibody fragments, that have an antigen-binding domain. Nucleic acids, molecules, vectors, and host cells useful in the production of the antibody products are also provided. The antibody products and compositions thereof can be used to prevent or treat a variety of different disease conditions including, but not limited to, preventing or treating disease conditions such as cancer. LILRB2/1 antibodies provided herein have a useful half-life and display anti-tumor activity in humanized-mouse models. Humanized LILRB2/1 antibodies provided herein restore effector function of activated and exhausted T cells from M2c-mediated immune suppression and enhance secretion of proinflammatory cytokines by M0 macrophages or LPS stimulated PBMCs.

It is believed that the LILRB2/1 antibody products provided herein bind LILRB1 and LILRB2 proteins at epitopes that are distinct from those bound by other antibodies currently in development or in the clinic. In preclinical studies, the antibody products enhanced LPS-induced IFN-γ production and reduced IL-10 release by peripheral blood mononuclear cells (PBMCs) and relieved immune suppression of pro-tumoral macrophages to induce activation of T cells, shown by enhanced proliferation, IFN-γ secretion and perforin release. A representative antibody product has demonstrated the ability to restore the ability of exhausted T cells to secrete IFN-γ in the presence of pro-tumoral macrophages and has also significantly enhanced the activity of pembrolizumab in combination studies. Importantly, a chimeric antibody product demonstrated superior anti-tumor activity over the comparator with significant tumor growth inhibition and tumor regression in a humanized mouse tumor model.

The LILRB2/1 antibody products do not cross-react with non-human primate LILRB2, show minimal binding to cynomolgus LILRB1, and show no cross reactivity to other inhibitory or activating LILR family members. Treatment with representative antibody products did not trigger inflammatory cytokine release in human whole blood.

Without wishing to be bound by any particular theory, it is contemplated herein that the immune stimulatory activity of the antibodies provided appears to arise from two distinct but coordinated mechanisms: variable region engagement of LILRB1/LILRB2 along with heavy chain engagement of Fc receptors.

On the one hand, it is believed that LILRB1 and LILRB2 expressed on myeloid cells negatively regulate anti-tumor immunity via binding to HLA-G on tumor cells and HLA Class 1 on myeloid cells. Binding of LILRB1 and LILRB2 to these ligands promotes immunosuppressive myeloid cells in the tumor microenvironment via inhibition of Ca²⁺ signaling across the cell membranes of the myeloid cells, recruitment of Src-homology domain 2 containing protein tyrosine phosphatase-1 (SHP1) and SHP2 phosphatases within the myeloid cell membranes, and cytokine production by the myeloid cells that can prevent checkpoint inhibitor (CPI) therapies from potentiating anti-tumor T cell responses. Individually or collectively, these phenomena can result in reduced killing of cancer cells. It is also believed that LILRB1 and LILRB2 further impede tumor cell killing by competing with cytotoxic T lymphocytes for binding to HLA class I.

On the other hand, activating Fc receptors (FcγRIA, FcγRIIA, and FcγRIIIA) on myeloid cells is known to play an essential role in promoting cell activation, differentiation, and induction of adaptive immune responses through the modulation of antigen presentation. These receptors signal through an immunoreceptor tyrosine-based activation motif via SRC family kinases and spleen tyrosine kinase leading to transcriptional activation of several pro-inflammatory cytokines and chemokines driving cell mobilization, migration, differentiation, and survival.

The antibodies disclosed herein appear to enable coupling of LILRB2/1 antagonism with Fc receptor-mediated activation. The antibodies bind a unique epitope on LILRB2 to not only block myeloid cell interactions with tumor cell HLA-G, but also cis-interactions with HLA class I, thereby deactivating both inhibitory signals in the myeloid cells. This action can promote the polarization of tumor-infiltrating myeloid cells toward an inflammatory phenotype, while simultaneously freeing MHC Class I on these cells to engage receptors required for optimal activation of cytotoxic T lymphocytes. Moreover, the antibodies can enhance anti-tumor immunity by providing an immunostimulatory signal through engagement of activating FcγRIIIA. This dual, or co-engagement, mechanism provides these antibodies with novel attributes for targeting myeloid cells to reverse CPI resistance, enhance tumor cell killing, and improve patient outcomes.

The disclosure provides an antibody product comprising a variable region that specifically binds human LILRB2 and LILRB1, the variable region comprising: a first domain comprising a CDR-H1 set forth in SEQ ID NO: 16, a CDR-H2 set forth in SEQ ID NO: 17, and a CDR-H3 set forth in SEQ ID NO: 18, and a second domain comprising a CDR-L1 set forth in SEQ ID NO: 19, a CDR-L2 set forth in SEQ ID NO: 20, and a CDR-L3 set forth in SEQ ID NO: 21, as specified using the IMGT CDR definition; or a first domain comprising a CDR-H1 set forth in SEQ ID NO: 22, a CDR-H2 set forth in SEQ NO: 23, and a CDR-H3 set forth in SEQ ID NO: 24, and a second domain comprising a CDR-L1 set forth in SEQ ID NO: 25, a CDR-L2 set forth in SEQ ID NO: 26, and a CDR-L3 set forth in SEQ ID NO: 27, as specified using the Kabat CDR definition.

The variable region can comprise a heavy chain variable domain comprising: an amino acid sequence at least 80% identical to SEQ ID NO: 1, 6, 7, 8, 9 or 10; or an amino acid sequence set forth in SEQ ID NO: 1, 6, 7, 8, 9 or 10.

The variable region can comprise a light chain variable domain comprising: an amino acid sequence at least 80% identical to SEQ ID NO: 2, 11, 12, 13, 14 or 15; or an amino acid sequence set forth in SEQ ID NO: 2, 11, 12, 13, 14 or 15.

The variable region can comprise: a heavy chain variable domain comprising SEQ ID NO: 1; and a light chain variable domain SEQ ID NO: 2; a heavy chain variable domain comprising SEQ ID NO: 6; and a light chain variable domain comprising SEQ ID NO: 11; a heavy chain variable domain comprising SEQ ID NO: 6; and a light chain variable domain comprising SEQ ID NO: 12; a heavy chain variable domain comprising SEQ ID NO: 6; and a light chain variable domain comprising SEQ ID NO: 13; a heavy chain variable domain comprising SEQ ID NO: 6; and a light chain variable domain comprising SEQ ID NO: 14; a heavy chain variable domain comprising SEQ ID NO: 6; and a light chain variable domain comprising SEQ ID NO: 15; a heavy chain variable domain comprising SEQ ID NO: 7; and a light chain variable comprising SEQ ID NO: 11; a heavy chain variable domain comprising SEQ ID NO: 7; and a light chain variable domain comprising SEQ ID NO: 12; a heavy chain variable domain comprising SEQ ID NO: 7; and a light chain variable domain comprising SEQ ID NO: 13; a heavy chain variable domain comprising SEQ ID NO: 7; a light chain variable domain comprising SEQ ID NO: 14; a heavy chain variable domain comprising SEQ ID NO: 7; and a light chain variable domain comprising SEQ ID NO: 15; a heavy chain variable domain comprising SEQ ID NO: 8; and a light chain variable domain comprising SEQ ID NO: 11; a heavy chain variable domain comprising SEQ ID NO: 8; and a light chain variable domain comprising SEQ ID NO: 12; a heavy chain variable domain comprising SEQ ID NO: 8; and a light chain variable domain comprising SEQ ID NO: 13; a heavy chain variable domain comprising SEQ ID NO: 8; and a light chain variable domain comprising SEQ ID NO: 14; a heavy chain variable domain comprising SEQ ID NO: 8; and a light chain variable domain comprising SEQ ID NO: 15; a heavy chain variable domain comprising SEQ ID NO: 9; a light chain variable domain comprising SEQ ID NO: 11; a heavy chain variable domain comprising SEQ ID NO: 9; and a light chain variable domain comprising SEQ ID NO: 12; a heavy chain variable domain comprising SEQ ID NO: 9; and a light chain variable domain comprising SEQ ID NO: 13; a heavy chain variable domain comprising SEQ ID NO: 9; and a light chain variable domain comprising SEQ ID NO: 14; a heavy chain variable domain comprising SEQ ID NO: 9; and a light chain variable domain comprising SEQ ID NO: 15; a heavy chain variable domain comprising SEQ ID NO: 10; and a light chain variable domain comprising SEQ ID NO: 11; a heavy chain variable domain comprising SEQ ID NO: 10; and a light chain variable domain comprising SEQ ID NO: 12; a heavy chain variable domain comprising SEQ ID NO: 10; and a light chain variable domain comprising SEQ ID NO: 13; a heavy chain variable domain comprising SEQ ID NO: 10; and a light chain variable domain comprising SEQ ID NO: 14; or a heavy chain variable domain comprising SEQ ID NO: 10; and a light chain variable domain comprising SEQ ID NO: 15.

An antibody product provided herein can comprise a heavy chain comprising a heavy chain variable domain (VH) and a human heavy chain constant domain (CH).

An antibody product provided herein can comprise a light chain comprising a light chain variable domain (VL) and a human light chain constant domain (CL).

An antibody product provided herein can comprise a heavy chain comprising a heavy chain variable domain (VH) and a human heavy chain constant domain (CH) and a light chain comprising a light chain variable domain (VL) and a human light chain constant domain (CL).

A heavy chain constant domain of an antibody product provided herein can comprise an IgA, IgD, IgE, IgG, or IgM heavy chain constant domain. The heavy chain constant domain can be an IgG1 constant domain, an IgG2 constant domain, or an IgG4 constant domain. The heavy chain constant domain can be an IgG1 constant domain. The heavy chain amino acid sequence can comprise: an amino acid sequence at least 80% identical to SEQ ID NO: 3, SEQ ID NO: 28, SEQ ID NO: 29, SEQ ID NO: 30, SEQ ID NO: 31, or SEQ ID NO: 32; or an amino acid sequence set forth in SEQ ID NO: 3, SEQ ID NO: 28, SEQ ID NO: 29, SEQ ID NO: 30, SEQ ID NO: 31, or SEQ ID NO: 32. The heavy chain constant domain can be an IgG4 constant domain. The heavy chain amino acid sequence can comprise: an amino acid sequence at least 80% identical to SEQ ID NO: 4, SEQ ID NO: 33, SEQ ID NO: 34, SEQ ID NO: 35, SEQ ID NO: 36, or SEQ ID NO: 37; or an amino acid sequence set forth in SEQ ID NO: 4, SEQ ID NO: 33, SEQ ID NO: 34, SEQ ID NO: 35, SEQ ID NO: 36, or SEQ ID NO: 37.

An antibody product provided herein can comprise a light chain comprising a light chain variable region (VL) and a human light chain constant region (CL).

A human light chain constant region (CL) can comprise a kappa domain or a fragment thereof. The light chain amino acid sequence can comprise: an amino acid sequence at least 80% identical to SEQ ID NO: 5, SEQ ID NO: 38, SEQ ID NO: 39, SEQ ID NO: 40, SEQ ID NO: 41, or SEQ ID NO: 42; or an amino acid sequence set forth in SEQ ID NO: 5, SEQ ID NO: 38, SEQ ID NO: 39, SEQ ID NO: 40, SEQ ID NO: 41, or SEQ ID NO: 42.

The disclosure provides an antibody product that binds human LILRB2, comprising: an IgG1 heavy chain having an amino acid sequence comprising SEQ ID NO: 1; and a kappa light chain having an amino acid sequence comprising SEQ ID NO: 2; an IgG1 heavy chain having an amino acid sequence comprising SEQ ID NO: 6; and a kappa light chain having an amino acid sequence comprising SEQ ID NO: 11; an IgG1 heavy chain having an amino acid sequence comprising SEQ ID NO: 6; and a kappa light chain having an amino acid sequence comprising SEQ ID NO: 12; an IgG1 heavy chain having an amino acid sequence comprising SEQ ID NO: 6; and a kappa light chain having an amino acid sequence comprising SEQ ID NO: 13; an IgG1 heavy chain having an amino acid sequence comprising SEQ ID NO: 6; and a kappa light chain having an amino acid sequence comprising SEQ ID NO: 14; an IgG1 heavy chain having an amino acid sequence comprising SEQ ID NO: 6; and a kappa light chain having an amino acid sequence comprising SEQ ID NO: 15; an IgG1 heavy chain having an amino acid sequence comprising SEQ ID NO: 7; and a kappa light chain having an amino acid sequence comprising SEQ ID NO: 11; an IgG1 heavy chain having an amino acid sequence comprising SEQ ID NO: 7; and a kappa light chain having an amino acid sequence comprising SEQ ID NO: 12; an IgG1 heavy chain having an amino acid sequence comprising SEQ ID NO: 7; and a kappa light chain having an amino acid sequence comprising SEQ ID NO: 13; an IgG1 heavy chain having an amino acid sequence comprising SEQ ID NO: 7; and a kappa light chain having an amino acid sequence comprising SEQ ID NO: 14; an IgG1 heavy chain having an amino acid sequence comprising SEQ ID NO: 7; and a kappa light chain having an amino acid sequence comprising SEQ ID NO: 15; an IgG1 heavy chain having an amino acid sequence comprising SEQ ID NO: 8; and a kappa light chain having an amino acid sequence comprising SEQ ID NO: 11; an IgG1 heavy chain having an amino acid sequence comprising SEQ ID NO: 8; and a kappa light chain having an amino acid sequence comprising SEQ ID NO: 12; an IgG1 heavy chain having an amino acid sequence comprising SEQ ID NO: 8; and a kappa light chain having an amino acid sequence comprising SEQ ID NO: 13; an IgG1 heavy chain having an amino acid sequence comprising SEQ ID NO: 8; and a kappa light chain having an amino acid sequence comprising SEQ ID NO: 14; an IgG1 heavy chain having an amino acid sequence comprising SEQ ID NO: 8; and a kappa light chain having an amino acid sequence comprising SEQ ID NO: 15; an IgG1 heavy chain having an amino acid sequence comprising SEQ ID NO: 9; and a kappa light chain having an amino acid sequence comprising SEQ ID NO: 11; an IgG1 heavy chain having an amino acid sequence comprising SEQ ID NO: 9; and a kappa light chain having an amino acid sequence comprising SEQ ID NO: 12; an IgG1 heavy chain having an amino acid sequence comprising SEQ ID NO: 9; and a kappa light chain having an amino acid sequence comprising SEQ ID NO: 13; an IgG1 heavy chain having an amino acid sequence comprising SEQ ID NO: 9; and a kappa light chain having an amino acid sequence comprising SEQ ID NO: 14; an IgG1 heavy chain having an amino acid sequence comprising SEQ ID NO: 9; and a kappa light chain having an amino acid sequence comprising SEQ ID NO: 15; an IgG1 heavy chain having an amino acid sequence comprising SEQ ID NO: 10; and a kappa light chain having an amino acid sequence comprising SEQ ID NO: 11; an IgG1 heavy chain having an amino acid sequence comprising SEQ ID NO: 10; and a kappa light chain having an amino acid sequence comprising SEQ ID NO: 12; an IgG1 heavy chain having an amino acid sequence comprising SEQ ID NO: 10; and a kappa light chain having an amino acid sequence comprising SEQ ID NO: 13; an IgG1 heavy chain having an amino acid sequence comprising SEQ ID NO: 10; and a kappa light chain having an amino acid sequence comprising SEQ ID NO: 14; or an IgG1 heavy chain having an amino acid sequence comprising SEQ ID NO: 10; and a kappa light chain having an amino acid sequence comprising SEQ ID NO: 15.

The disclosure provides an antibody product that binds human LILRB2, comprising: an IgG4 heavy chain having an amino acid sequence comprising SEQ ID NO: 1; and a kappa light chain having an amino acid sequence comprising SEQ ID NO: 2; an IgG4 heavy chain having an amino acid sequence comprising SEQ ID NO: 6; and a kappa light chain having an amino acid sequence comprising SEQ ID NO: 11; an IgG4 heavy chain having an amino acid sequence comprising SEQ ID NO: 6; and a kappa light chain having an amino acid sequence comprising SEQ ID NO: 12; an IgG4 heavy chain having an amino acid sequence comprising SEQ ID NO: 6; and a kappa light chain having an amino acid sequence comprising SEQ ID NO: 13; an IgG4 heavy chain having an amino acid sequence comprising SEQ ID NO: 6; and a kappa light chain having an amino acid sequence comprising SEQ ID NO: 14; an IgG4 heavy chain having an amino acid sequence comprising SEQ ID NO: 6; and a kappa light chain having an amino acid sequence comprising SEQ ID NO: 15; an IgG4 heavy chain having an amino acid sequence comprising SEQ ID NO: 7; and a kappa light chain having an amino acid sequence comprising SEQ ID NO: 11; an IgG4 heavy chain having an amino acid sequence comprising SEQ ID NO: 7; and a kappa light chain having an amino acid sequence comprising SEQ ID NO: 12; an IgG4 heavy chain having an amino acid sequence comprising SEQ ID NO: 7; and a kappa light chain having an amino acid sequence comprising SEQ ID NO: 13; an IgG4 heavy chain having an amino acid sequence comprising SEQ ID NO: 7; and a kappa light chain having an amino acid sequence comprising SEQ ID NO: 14; an IgG4 heavy chain having an amino acid sequence comprising SEQ ID NO: 7; and a kappa light chain having an amino acid sequence comprising SEQ ID NO: 15; an IgG4 heavy chain having an amino acid sequence comprising SEQ ID NO: 8; and a kappa light chain having an amino acid sequence comprising SEQ ID NO: 11; an IgG4 heavy chain having an amino acid sequence comprising SEQ ID NO: 8; and a kappa light chain having an amino acid sequence comprising SEQ ID NO: 12; an IgG4 heavy chain having an amino acid sequence comprising SEQ ID NO: 8; and a kappa light chain having an amino acid sequence comprising SEQ ID NO: 13; or an IgG4 heavy chain having an amino acid sequence comprising SEQ ID NO: 8; and a kappa light chain having an amino acid sequence comprising SEQ ID NO: 14.

An antibody product provided herein can be a monoclonal antibody, a human antibody, a chimeric antibody, a humanized antibody, or a single chain antibody.

An antibody product provided herein can be a monospecific, bispecific, trispecific, or multispecific antibody.

An antibody product provided herein can specifically bind human LILRB2 expressed by myeloid cells or cells of a cancer. The antibody product can specifically bind to human LILRB2 with a K_{D} below 1 pM to about 1000 pM. The antibody product can specifically bind human LILRB1 expressed by myeloid cells, lymphocytes, or cells of a cancer. The antibody product can specifically bind to human LILRB1 with a K_{D} from about 0.1 pM to about 300 nM.

An antibody product provided herein can bind to a human immunosuppressive myeloid cell. The antibody product can bind to a human immunosuppressive myeloid cell in a tumor microenvironment. The immunosuppressive myeloid cell can be a macrophage, a myeloid dendritic cell, or a myeloid-derived suppressor cell. The immunosuppressive myeloid cell can be an M2a, M2b, M2c, or M2d macrophage. The antibody product can specifically bind to human M2c macrophages with a K_{D} from 0.05 nM to 50 nM. Binding of the antibody product to the immunosuppressive myeloid cell may or may not be cytotoxic to the cell as needed.

An antibody product provided herein can bind to a human lymphocyte. The antibody product can bind to a human lymphocyte in a tumor microenvironment. The lymphocyte can be a human B lymphocyte, a human T lymphocyte, or an NK cell. The antibody product can specifically bind to human lymphocytes with a K_{D} from 0.001 nM to 300 nM. Binding of the antibody product to a lymphocyte may or may not be cytotoxic to the lymphocyte as needed.

An antibody product provided herein can inhibit immunosuppressive interaction of LILRB1 or LILRB2 expressed on a myeloid cell with HLA-G expressed on a cell in the tumor microenvironment, such as a tumor cell, cancer-associated fibroblast, other immune-suppressive myeloid cell, or lymphocyte.

An antibody product provided herein can inhibit immunosuppressive interaction of LILRB1 expressed on a lymphocyte with HLA-G expressed on a tumor cell, cancer-associated fibroblast as well as immune suppressive myeloid cells or lymphocytes.

An antibody product provided herein can inhibit immunosuppressive interaction of LILRB1 or LILRB2 expressed on a myeloid cell with HLA class I expressed on the same myeloid cell, or on a tumor cell, cancer-associated fibroblast, other immune-suppressive myeloid cell, or lymphocyte.

An antibody product provided herein can inhibit immunosuppressive interaction of LILRB1 expressed on a lymphocyte with HLA-class I expressed on the same lymphocyte or on a tumor cell, cancer-associated fibroblast, other immune-suppressive myeloid cell, or lymphocyte.

An antibody product provided herein can be bound by an Fc receptor expressed on an immunosuppressive macrophage or other myeloid cell or a lymphocyte. The antibody product can be bound by CD16 (FcγRIIIa), CD32 (FcγRII), or CD64 (FcγRI) expressed on an immunosuppressive macrophage or other myeloid cell or a lymphocyte. The antibody product can bind to LILRB1 or LILRB2 expressed on the myeloid cell and is bound by CD16 (FcγRIIIa), CD32 (FcγRII), or CD64 (FcγRI) expressed on the same myeloid cell. The antibody product can bind to LILRB1 expressed on the lymphocyte and is bound by CD16 (FcγRIIIa), CD32 (FcγRII), or CD64 (FcγRI) expressed on the same lymphocyte. The antibody product can bind to LILRB1 or LILRB2 expressed on a first cell and is bound by CD16 (FcγRIIIa), CD32 (FcγRII), or CD64 (FcγRI) expressed on a second cell.

The disclosure provides a method of cancer immunotherapy for a subject in need thereof, in which the cancer is associated with a presence of immunosuppressive macrophages, the method comprising administering to the subject a therapeutically effective amount of a pharmaceutical composition comprising an antibody product provided herein. The method can comprise administering an amount of the pharmaceutical composition effective to inhibit immunosuppressive interaction of LILRB1 or LILRB2 expressed on a myeloid cell with HLA-G or HLA class I expressed on the myeloid cell or on a tumor cell, cancer-associated fibroblast, other immune-suppressive myeloid cell, or lymphocyte. The method can comprise administering an amount of the pharmaceutical composition effective to inhibit immunosuppressive interaction of LILRB1 expressed on a lymphocyte with HLA-G or HLA-class I expressed by the lymphocytes or on a tumor cell, cancer-associated fibroblast, other immune-suppressive myeloid cell, or lymphocyte. The antibody product can bind to a macrophage and binding of the antibody product to a macrophage can result in at least one of the following effects: promotes activation of a CD4⁺ T cell, CD8⁺ T cell, NK cell, or any combination thereof; promotes proliferation of a CD4⁺ T cell, CD8⁺ T cell, NK cell, or any combination thereof; prevents polarization of macrophages to immunosuppressive macrophages; and enhances an innate anti-tumor response. The activation of a CD4⁺ T cell, CD8⁺ T cell, NK cell, or any combination thereof can be measured as an enhanced level of IFN-γ, TNF-α, or perforin, or any combination thereof. Binding of the antibody product to a macrophage may or may not be cytotoxic to the macrophage as needed. Binding of the antibody product to a macrophage can result in: internalization of the antibody product by the macrophage; secretion of TNFα, IL-6, perforin, or any combination thereof; reduced secretion of IL-10; activation of a CD4⁺ T cell, CD8⁺ T cell, NK cell, or any combination thereof; proliferation of a CD4⁺ T cell, CD8⁺ T cell, NK cell, or any combination thereof; or promotion of tumor cell killing in a tumor microenvironment.

In cancer immunotherapy methods provided herein, binding of the antibody product to a macrophage can increase an immunostimulatory activity in a tumor microenvironment.

In cancer immunotherapy methods provided herein, binding of the antibody product to a macrophage can reduce an immunosuppressive activity of the macrophage.

In cancer immunotherapy methods provided herein, binding of the antibody product to a macrophage can reduce a tumor-promoting activity of the macrophage.

In cancer immunotherapy methods provided herein, binding of the antibody product to a macrophage can promote CD4⁺ T cell activation, CD4⁺ T cell proliferation, or both CD4⁺ T cell activation and proliferation.

In cancer immunotherapy methods provided herein, binding of the antibody product to a macrophage can promote CD8⁺ T cell activation, CD8⁺ T cell proliferation, or both CD8⁺ T cell activation and proliferation.

In cancer immunotherapy methods provided herein, binding of the antibody product to a macrophage can promote cytotoxic lymphocyte-mediated killing of cancer cells.

In cancer immunotherapy methods provided herein, binding of the antibody product to a macrophage can promote NK cell-mediated tumor cell killing.

In cancer immunotherapy methods provided herein, binding of the antibody product can reduce suppression of cytotoxic T cell-mediated killing of tumor cells in the tumor microenvironment.

In cancer immunotherapy methods provided herein in which the antibody product binds to a lymphocyte, binding of the antibody product to a lymphocyte can: promotes NK cell-mediated killing of tumor cells and enhances IFN-γ secretion by and cytotoxic activity of CD8⁺ T cells; activates NK cells and CD8⁺ T cells; or blocks LILRB1 mediated suppression of lymphocytes.

In cancer immunotherapy methods provided herein, the cancer can be, for example, a sarcoma, carcinoma, or blood-borne cancer. The cancer can glioblastoma multiforme, head and neck cancer, kidney renal clear cell cancer, pancreatic adenocarcinoma, skin cutaneous melanoma, stomach adenocarcinoma, testicular germ cell cancer, gastric cancer, Merkel cell carcinoma, dendritic sarcoma, non-small cell lung cancer, papillary thyroid cancer, cutaneous squamous cell carcinoma, or ovarian cancer. The cancer can be a B-cell malignancy selected from among B-cell lymphoma (such as diffuse large B-cell lymphoma, small lymphocytic lymphoma or chronic lymphocytic leukemia, non-Hodgkin lymphoma, mantle cell lymphoma, marginal zone lymphoma, Burkitt lymphoma, follicular lymphoma, lymphoplasmacytic lymphoma), B cell leukemia, acute myeloid leukemia, and multiple myeloma.

In cancer immunotherapy methods provided herein, the cells of the cancer express LILRB2 or LILRB1. The cells of the cancer can overexpress LILRB2 or LILRB1.

In cancer immunotherapy methods provided herein, the methods can further comprise administering to the subject an effective amount of an anti-cancer therapeutic product. The anti-cancer therapeutic product can comprise an immune checkpoint inhibitor. The immune checkpoint inhibitor can be administered in an amount effective to relieve immunosuppression of T cells. The immune checkpoint inhibitor can be a PD-1 antagonist, PD-L1 antagonist, or CLTA-4 antagonist. The immunosuppression of T cells can comprise immunosuppression mediated through interaction of the T cells with myeloid cells expressing PD-L1.

The disclosure provides an isolated nucleic acid, comprising a nucleotide sequence encoding a heavy chain variable domain provided herein.

The disclosure provides an isolated nucleic acid, comprising a nucleotide sequence encoding a light chain variable domain provided herein.

The disclosure provides an expression vector comprising a nucleic acid provided herein.

The disclosure provides a host cell comprising a nucleic acid or expression vector provided herein.

The disclosure provides a method of producing a protein comprising an immunoglobulin heavy chain variable domain or an immunoglobulin light chain variable domain. Such a method can comprise: growing a host cell provided herein encoding the antibody product under conditions such that the host cell expresses the protein comprising the immunoglobulin heavy chain variable domain or the immunoglobulin light chain variable domain; and purifying the protein comprising the immunoglobulin heavy chain variable domain or the immunoglobulin light chain variable domain.

The disclosure provides a method of producing an antibody product that binds human LILRB2. Such a method can comprise: growing a host cell comprising an expression vector provided herein encoding the antibody product under conditions such that the host cell expresses a protein comprising the immunoglobulin heavy chain variable domain and the immunoglobulin light chain variable domain, thereby producing the antibody product; and purifying the antibody product.

The disclosure provides a composition comprising an antibody product provided herein and an excipient.

The disclosure provides an article of manufacture, comprising the composition provided herein and a container.

The disclosure provides a use of the antibody product or the composition provided herein, for manufacture of a medicament for treatment of cancer in a subject in need thereof.

The disclosure provides a pharmaceutical composition, comprising an antibody product provided herein and a pharmaceutically acceptable excipient.

The disclosure provides an antibody product or pharmaceutical composition provided herein, for use in treating a subject having a cancer expressing LILRB2.

The disclosure provides a use of an antibody product in the manufacture of a medicament for treating a subject having a cancer expressing LILRB2.

The disclosure provides a method of detecting LILRB2 in a sample, tissue, or cell using the antibody product provided herein, comprising contacting the sample, tissue or cell with the antibody product and detecting the antibody product.

The disclosure provides a method of reducing a biological activity of LILRB1 or LILRB2 in a subject in need thereof, comprising administering a therapeutically effective amount of the antibody product or pharmaceutical composition provided herein. The antibody product can mediate depletion of at least one cancer cell expressing LILRB2.

The disclosure provides a method of promoting an immune response in a subject in need thereof, comprising administering a therapeutically effective amount of the antibody product or pharmaceutical composition provided herein.

The disclosure provides a method of cancer immunotherapy for a subject in need thereof, in which cells of the cancer express LILRB2, the method comprising administering to the subject a therapeutically effective amount of an antibody product provided herein.

The method of 90, comprising administering an amount the antibody product effective to mediate killing of cells of the cancer through antibody-dependent cellular toxicity. The method can comprise administering an amount of the antibody product effective to relieve LILRB1- or LILRB2-mediated suppression of T cells in the subject. The method can further comprise administering to the subject a PD-1 antagonist or a PD-L1 antagonist in an amount sufficient to relieve PD-1/PD-L1 axis-mediated immunosuppression of T cells in the subject, or a CTLA-4 antagonist in an amount sufficient to relieve CTLA-4 axis-mediated immunosuppression of T cells in the subject. The PD-1 antagonist can comprise a PD-1 antibody product. The PD-L1 antagonist can comprise a PD-L1 antibody product. The CTLA-4 antagonist can comprise a CTLA-4 antibody product.

An antibody product provided herein can be detectably labeled or comprise a conjugated toxin, drug, receptor, enzyme, receptor ligand. The antibody product can comprise a therapeutic or cytotoxic moiety.

A pharmaceutical composition provided herein can comprise an antibody product provided herein that reduces or prevents binding of LILRB1 or LILRB2 to a ligand thereof and/or reduces or prevents LILRB1- or LILRB2-mediated signal transduction and a physiologically acceptable carrier or excipient. The ligand can be, for example, a human leukocyte antigen A, human leukocyte antigen B, human leukocyte antigen C, human leukocyte antigen G, angiopoietin-like protein 2, angiopoietin-like protein 5, or a combination thereof. The ligand can be expressed on the surface of a myeloid cell, a lymphocyte, or a tumor cell.

A method of treating cancer or a tumor in a subject in need thereof provided herein can comprise administering to the subject an effective amount of a pharmaceutical composition provided herein. The subject can have cancer or a tumor comprising cells expressing or over-expressing a ligand of LILRB2. The antibody product or antigen binding fragment thereof in the pharmaceutical composition increases an immune response, retards or prevents tumor growth, inhibits tumor-mediated immune suppression, eliminate tumors, depletes or blocks the activity of tumor-associated macrophages so as to alter their activity, decreases tumor-associated macrophage-mediated immune suppression, reduces or reverses T cell suppression, or a combination thereof. The cancer or tumor can comprise macrophages expressing LILRB2. The method can further comprise administering to the subject a therapeutic amount of a second therapeutic product. The second therapeutic product can comprise an immune checkpoint inhibitor. The second therapeutic product can comprise a PD-1 antagonist, PD-L1 antagonist, or CTLA-4 antagonist.

An antibody product provided herein can comprise: a heavy chain comprising SEQ ID NO: 1; and a light chain comprising SEQ ID NO: 2; a heavy chain comprising SEQ ID NO: 6; and a light chain comprising SEQ ID NO: 11; a heavy chain comprising SEQ ID NO: 6; and a light chain comprising SEQ ID NO: 12; a heavy chain comprising SEQ ID NO: 6; and a light chain comprising SEQ ID NO: 13; a heavy chain comprising SEQ ID NO: 6; and a light chain comprising SEQ ID NO: 14; a heavy chain comprising SEQ ID NO: 6; and a light chain comprising SEQ ID NO: 15; a heavy chain comprising SEQ ID NO: 7; and a light chain variable comprising SEQ ID NO: 11; a heavy chain comprising SEQ ID NO: 7; and a light chain comprising SEQ ID NO: 12; a heavy chain comprising SEQ ID NO: 7; and a light chain comprising SEQ ID NO: 13; a heavy chain comprising SEQ ID NO: 7; a light chain comprising SEQ ID NO: 14; a heavy chain comprising SEQ ID NO: 7; and a light chain comprising SEQ ID NO: 15; a heavy chain comprising SEQ ID NO: 8; and a light chain comprising SEQ ID NO: 11; a heavy chain comprising SEQ ID NO: 8; and a light chain comprising SEQ ID NO: 12; a heavy chain comprising SEQ ID NO: 8; and a light chain comprising SEQ ID NO: 13; a heavy chain comprising SEQ ID NO: 8; and a light chain comprising SEQ ID NO: 14; a heavy chain comprising SEQ ID NO: 8; and a light chain comprising SEQ ID NO: 15; a heavy chain comprising SEQ ID NO: 9; a light chain comprising SEQ ID NO: 11; a heavy chain comprising SEQ ID NO: 9; and a light chain comprising SEQ ID NO: 12; a heavy chain comprising SEQ ID NO: 9; and a light chain comprising SEQ ID NO: 13; a heavy chain comprising SEQ ID NO: 9; and a light chain comprising SEQ ID NO: 14; a heavy chain comprising SEQ ID NO: 9; and a light chain comprising SEQ ID NO: 15; a heavy chain comprising SEQ ID NO: 10; and a light chain comprising SEQ ID NO: 11; a heavy chain comprising SEQ ID NO: 10; and a light chain comprising SEQ ID NO: 12; a heavy chain comprising SEQ ID NO: 10; and a light chain comprising SEQ ID NO: 13; a heavy chain comprising SEQ ID NO: 10; and a light chain comprising SEQ ID NO: 14; or a heavy chain comprising SEQ ID NO: 10; and a light chain comprising SEQ ID NO: 15.

An antibody product provided herein can comprise: a heavy chain comprising SEQ ID NO: 1; and a light chain comprising SEQ ID NO: 2; a heavy chain comprising SEQ ID NO: 28 or SEQ ID NO: 33; and a light chain comprising SEQ ID NO: 38; a heavy chain comprising SEQ ID NO: 28 or SEQ ID NO: 33; and a light chain comprising SEQ ID NO: 39; a heavy chain comprising SEQ ID NO: 28 or SEQ ID NO: 33; and a light chain comprising SEQ ID NO: 40; a heavy chain comprising SEQ ID NO: 28 or SEQ ID NO: 33; and a light chain comprising SEQ ID NO: 41; a heavy chain comprising SEQ ID NO: 28 or SEQ ID NO: 33; and a light chain comprising SEQ ID NO: 42; a heavy chain comprising SEQ ID NO: 29 or SEQ ID NO: 34; and a light chain comprising SEQ ID NO: 38; a heavy chain comprising SEQ ID NO: 29 or SEQ ID NO: 34; and a light chain comprising SEQ ID NO: 39; a heavy chain comprising SEQ ID NO: 29 or SEQ ID NO: 34; and a light chain comprising SEQ ID NO: 40; a heavy chain comprising SEQ ID NO: 29 or SEQ ID NO: 34; and a light chain comprising SEQ ID NO: 41; a heavy chain comprising SEQ ID NO: 29 or SEQ ID NO: 34; and a light chain comprising SEQ ID NO: 42; a heavy chain comprising SEQ ID NO: 30 or SEQ ID NO: 35; and a light chain comprising SEQ ID NO: 38; a heavy chain comprising SEQ ID NO: 30 or SEQ ID NO: 35; and a light chain comprising SEQ ID NO: 39; a heavy chain comprising SEQ ID NO: 30 or SEQ ID NO: 35; and a light chain comprising SEQ ID NO: 40; a heavy chain comprising SEQ ID NO: 30 or SEQ ID NO: 35; and a light chain comprising SEQ ID NO: 41; a heavy chain comprising SEQ ID NO: 30 or SEQ ID NO: 35; and a light chain comprising SEQ ID NO: 42; a heavy chain comprising SEQ ID NO: 31 or SEQ ID NO: 36; and a light chain comprising SEQ ID NO: 38; a heavy chain comprising SEQ ID NO: 31 or SEQ ID NO: 36; and a light chain comprising SEQ ID NO: 39; a heavy chain comprising SEQ ID NO: 31 or SEQ ID NO: 36; and a light chain comprising SEQ ID NO: 40; a heavy chain comprising SEQ ID NO: 31 or SEQ ID NO: 36; and a light chain comprising SEQ ID NO: 41; a heavy chain comprising SEQ ID NO: 31 or SEQ ID NO: 36; and a light chain comprising SEQ ID NO: 42; a heavy chain comprising SEQ ID NO: 32 or SEQ ID NO: 37; and a light chain comprising SEQ ID NO: 38; a heavy chain comprising SEQ ID NO: 32 or SEQ ID NO: 37; and a light chain comprising SEQ ID NO: 39; a heavy chain comprising SEQ ID NO: 32 or SEQ ID NO: 37; and a light chain comprising SEQ ID NO: 40; a heavy chain comprising SEQ ID NO: 32 or SEQ ID NO: 37; and a light chain comprising SEQ ID NO: 41; or a heavy chain comprising SEQ ID NO: 32 or SEQ ID NO: 37; and a light chain comprising SEQ ID NO: 42.

The following Drawings and Detailed Description (including the Examples) illustrate various non-limiting aspects of the subject matter contemplated herein.

### BRIEF DESCRIPTION OF THE DRAWINGS

Aspects of the disclosure are illustrated by the following figures.

FIG. 1A-1B: LILRB2/1 chimera and humanized variant binding to LILRB1 expressing 721.221 B lymphoma and HEK-293 cells expressing LILRB2

FIG. 2A-2B: Blocking of LILRB2-Fc and LILRB1-Fc binding to HLA-G with LILRB2/1 chimera IgG1 and humanized variants

FIG. 3A-3C: Binding of LILRB2/1 antibodies to human monocytes, M0, M2c macrophages

FIG. 4A-4H: LILRB2/1 antibody binding to human monocyte and lymphocytes populations of PBMC from three subjects

FIG. 5A-5D: IFN-γ secretion by PBMC (from four subjects) stimulated with LPS following treatment with anti-LILRB2/1 chimera IgG1 and humanized variants in a dose titration

FIG. 6A-6B: LILRB2/1 Antibody effect on TNF-α secretion in CD40-activated macrophages (from two subjects)

FIG. 7A-7B: Rescue of (A) CD8⁺ T cell proliferation and (B) IFN-γ secretion with humanized variants (from three subjects) in M2c and CD8⁺ T cell coculture assay

FIG. 8A-8C: Rescue of IFN-γ release by exhausted T cells (from three subjects) from M2c-mediated immune suppression by LILRB2/1 chimera and humanized variants

FIG. 9A-9B: Enhancement of NK cell cytotoxicity by LILRB2/1 chimera IgG1 and humanized variants

FIG. 10A-10H: LILRB2/1 chimera and humanized variants induction of IFN-γ and IL-6 release in whole blood from healthy subjects

FIG. 11A-11C: LILRB2/1 antibody binding to myeloid cell subsets and lymphocytes (from three subjects)

FIG. 12A-12C: Pharmacokinetic profiling of LILRB2/B1 chimera and humanized variants in FcRn mice

FIG. 13: Anti-tumor efficacy of chimera LILRB2/1 antibody in humanized NSG-SGM3 mice and subcutaneous SK-MEL-5 human melanoma tumor model

FIG. 14A-14B: LILRB2/1 humanized antibody variants relieve HLA-G-mediated suppression of phagocytosis of tumor cells by M2 macrophages.

### DETAILED DESCRIPTION

Unless otherwise defined herein, scientific and technical terms used herein have the meanings that are commonly understood by those of ordinary skill in the art to which the claimed subject matter belongs.

### Antibody Products

The disclosure provides antibody products that specifically bind to certain human LILRB proteins, particularly LILRB1 and LILRB2, relative to other LILR family members.

“LILRB2/1 antibody product” means an antibody or fragment thereof that binds to LILRB1 and LILRB2, possibly with variable affinities between family members, but specifically relative to binding to other LILR family members. In some embodiments, LILRB2/1 antibody products bind with affinities that allow the antibody product to block or substantially impair binding of LILRB1 and LILRB2 to natural ligands, or to modulate activities mediated by LILRB1 and LILRB2. LILRB2/1 antibodies can be capable of LILRB2 antagonism and LILRB1 antagonism. As such, they may be referred to as “LILRB2/1 antagonists.”

“LILRB1 antagonist” means any chemical compound or biological molecule that blocks or substantially impairs binding of LILRB1 to any of its natural ligands, including, for example, HLA-A, —B, —C, —F, and -G, and HLA-B27. LILRB1 antagonists may block or impair interaction with ligands on another cell, thereby blocking or inhibiting functional activity associated with such “trans” interactions. LILRB1 antagonists may block or impair interaction with ligands on the same cell, thereby blocking or inhibiting functional activity associated with such “cis” interactions. An antibody that specifically binds LILRB1 can be an LILRB1 antagonist. Accordingly, a “LILRB1 antibody” or “LILRB1 antibody product” binds LILRB1 specifically relative to binding to other LILR family members.

“LILRB2 antagonist” means any chemical compound or biological molecule that blocks or substantially impairs binding of LILRB2 to any of its natural ligands, including, for example, members of the human leukocyte antigen family (including HLA-G, HLA-A, HLA-B, HLA-F) or an angiopoietin-like protein (ANGPTL, such as ANGPTL2, or ANGPTL5). LILRB2 antagonists may block or impair interaction with ligands on another cell, thereby blocking or inhibiting functional activity associated with such “trans” interactions. LILRB2 antagonists may block or impair interaction with ligands on the same cell, thereby blocking or inhibiting functional activity associated with such “cis” interactions. An antibody that specifically binds LILRB2 can be an LILRB2 antagonist. Accordingly, a “LILRB2 antibody” or “LILRB2 antibody product” binds LILRB1 specifically relative to binding to other LILR family members.

Detailed and reviewed genetic information for human LILRB2 is readily available, e.g., at www.ncbi.nlm.nih.gov/gene/10288.

Detailed and reviewed genetic information for human LILRB1 is readily available, e.g., at www.ncbi.nlm.nih.gov/gene/10859.

The terms “polypeptide” and “protein” are used interchangeably herein in the conventional way to refer to a molecule formed of amino acids. The polypeptides are not limited to a specific length. Peptides are included within polypeptides, unless specifically indicated otherwise. The terms neither specify nor exclude post-expression modifications of the polypeptide, for example, glycosylation, acetylation, phosphorylation, and the like, as well as other modifications known in the art, both naturally occurring and non-naturally occurring. Polypeptides of interest in the context of the antibodies of this disclosure include, but are not limited to, polypeptide fragments comprising CDRs which are capable of binding LILRB1 or LILRB2 proteins expressed by myeloid cells, lymphoid cells, or cancer cells.

The term “polypeptide fragment” refers to a polypeptide that has an amino-terminal deletion, a carboxyl-terminal deletion, and/or an internal deletion as compared with the full-length native protein. Such fragments can also contain modified amino acids as compared with the native protein. Fragments are about 5 to 500 amino acids long. For example, fragments can be at least 5, 6, 8, 10, 14, 20, 50, 70, 100, 110, 150, 200, 250, 300, 350, 400, or 450 amino acids long. Polypeptide fragments include immunologically functional fragments of antibodies, including binding domains. In the case of LILRB2/1 antibodies disclosed herein, useful fragments include, but are not limited, to a CDR region, a variable domain of a heavy or light chain, a portion of an antibody chain or just its variable region including two CDRs, and the like.

The term “isolated protein” referred to herein means that a subject protein (1) is free of at least some other proteins with which it would normally be found, (2) is essentially free of other proteins from the same source, (3) is expressed by a cell from a different species, (4) has been separated from at least about 50 percent of polynucleotides, lipids, carbohydrates, or other materials with which it is associated in nature, (5) is operably associated (by covalent or noncovalent interaction) with a polypeptide with which it is not associated in nature, or (6) does not occur in nature. Genomic DNA, cDNA, mRNA, or other RNA, of synthetic origin, or any combination thereof can encode such an isolated protein. Preferably, the isolated protein is substantially free from proteins or polypeptides or other contaminants that are found in its natural environment that would interfere with its therapeutic, diagnostic, prophylactic, research or other use.

A “variant” of a polypeptide (e.g., an antibody) comprises an amino acid sequence in which one or more amino acid residues are inserted into, deleted from and/or substituted into the amino acid sequence relative to another polypeptide sequence. Variants include fusion proteins.

A “derivative” of a polypeptide is a polypeptide (e.g., an antibody) that has been chemically modified in some manner distinct from insertion, deletion, or substitution variants, e.g., via conjugation to another chemical moiety.

The term “antibody” generally includes an immunoglobulin protein that comprises one or more polypeptide chains and that is immunologically functional, including specifically binding to an antigen. In humans, antibodies typically comprise four linked polypeptide chains, a “tetramer” including two identical “heavy” chains and two identical smaller “light” chains. The two heavy chains are each linked to one light chain, are also linked to one another in parallel. The linkages impart a roughly Y-shaped structure to the antibody, such that the linked portions of the heavy chains form the leg of the “Y”, and each light chain (with the portion of the heavy chain to which it is linked) forms an arm of the “Y”. Each arm of the antibody contains an antigen binding site, so the typical antibody can bind two of antigens. In humans there exist five basic types or classes of antibodies, differentiated by the structure of the heavy regions and by their functional purpose: IgG, IgA, IgE, IgD, IgM. Intact antibodies in some classes in humans can differ from the typical tetrameric “Y” structural unit, such as circulating IgM antibodies that contain five such units linked at their bases in a roughly circular array. More detail on antibody structure and function is provided elsewhere herein.

In a typical antibody, each pair or couplet in the tetrameric unit includes one full-length “light” chain (about 25 kDa) and one full-length “heavy” chain (about 50-70 kDa). Each individual immunoglobulin chain is composed of several “immunoglobulin domains,” each consisting of roughly 90 to 110 amino acids and expressing a characteristic folding pattern. These domains are the basic units of which antibody polypeptide chains are composed. The amino-terminal portion of each chain typically includes a variable domain that is responsible for antigen recognition. The carboxy-terminal portion is more conserved evolutionarily than the amino-terminal end of the chain and is referred to as the “constant region” or “C region.”

The term “heavy chain” includes a full-length immunoglobulin heavy chain and fragments thereof having sufficient variable domain sequence to confer binding specificity, either alone or together with a light chain variable domain. Heavy chains are typically classified as mu (μ), delta (δ), gamma (γ), alpha (α), or epsilon (ε) chains, and these define the antibody's isotype as IgM, IgD, IgG, IgA, and IgE, respectively. IgG has several subtypes, including, but not limited to, IgG1, IgG2, IgG3, and IgG4. IgM subtypes include IgM, and IgM2. IgA subtypes include IgA1 and IgA2. In humans, the IgA and IgD isotypes contain four heavy chains and four light chains; the IgG and IgE isotypes contain two heavy chains and two light chains; and the IgM isotype contains five heavy chains and five light chains. The heavy chain C region typically comprises one or more domains that can be responsible for effector function. The number of heavy chain constant region domains will depend on the isotype. Full-length IgG heavy chains, for example, each contain three C region domains known as CH1, CH2 and CH3, with the CH3 being closest to the carboxy terminus. The antibody products that are provided can have any of these isotypes and subtypes. For example, an LILRB2/1 antibody product can be an intact antibody of the IgG1 or IgG4 subtype.

The term “light chain” includes a full-length immunoglobulin light chain and fragments thereof having sufficient variable domain sequence to confer binding specificity, either alone or together with a heavy chain variable domain. Human light chains generally are classified as kappa (κ) or lambda (λ) light chains. A full-length light chain includes an amino-terminal variable domain (VL) and a carboxy terminal constant domain (CL).

In the light and heavy chains, the variable and constant domains are naturally joined by a “J” region of about twelve or more amino acids, with the heavy chain further including a “D” region of about ten more amino acids. See, e.g., Fundamental Immunology, 2nd ed., Ch. 7 (Paul, ed.) 1989, New York: Raven Press.

Variable domains of immunoglobulin chains generally exhibit the same overall structure, comprising relatively conserved framework regions (FR) joined by three hypervariable regions, more often called “complementarity determining regions” or CDRs. The CDRs from the two chains of each heavy chain/light chain pair mentioned above typically are aligned by the framework regions to form a structure that binds specifically with a particular epitope on the target protein (e.g., LILRB2 or LILRB1). From N-terminal to C-terminal, naturally occurring light and heavy chain variable regions both typically conform with the following order of these elements: FR1, CDR1, FR2, CDR2, FR3, CDR3 and FR4. Numbering systems have been devised for assigning numbers to amino acids that occupy positions in each of these domains.

The current art utilizes various numbering schemes with different definitions of CDR lengths and positions. For example, the Kabat numbering scheme is based on sequence alignment and uses “variability parameter” of a given amino acid position (the number of different amino acids at a given position divided by the frequency of the most occurring amino acid at that position) to predict CDRs [Kabat et al. in Sequences of Proteins of Immunological Interest, 5th Ed., US Dept. of Health and Human Services, PHS, NIH, NIH Pub. no. 91-3242 (1991)]. The Chothia numbering scheme, on the other hand, is a structure-based numbering scheme where antibody crystal structures are aligned as define the loop structures as CDRs [Chothia and Lesk, J Mol Biol. 1987 196:901-17; Chothia et al., Nature. 1989 342:878-83]. The Martin numbering scheme focuses on the structure alignment of different framework regions of unconventional lengths [Martin, “Protein Sequence and Structure Analysis of Antibody Variable Domains,” in: Kontermann and Dubel, eds. Antibody Engineering. Springer; Berlin, Germany: 2014. pp. 33-51]. The ImMunoGeneTics (IMGT) numbering scheme is a standardized numbering system based on alignments of sequences from a complete reference gene database including the whole immunoglobulin superfamily [Lefranc et al., Dev Comp Immunol. 2003 27(1):55-77; (www.imgt.org/IMGTScientificChart/Nomenclature/IMGT-FRCDRdefinition.html)]. The Honneger numbering scheme (AHo) is based on structural alignments of the 3D structure of the variable regions and uses structurally conserved Ca positions to deduce framework and CDR lengths [Honegger et al., J Mol Biol. 2001 309(3):657-70]. One of ordinary skill in the art understands that the definition of a CDR will vary based on the method used.

Some of the antibody products that are provided have the structure typically associated with naturally occurring antibodies. Thus, the term “antibody product” includes an intact antibody of any class or subclass, or a fragment thereof that can compete with the intact antibody for specific binding to the target antigen, and includes chimeric, humanized, fully human, and bispecific antibodies, as well as other forms. As noted, an intact antibody generally will comprise at least two full-length heavy chains and two full-length light chains, but in some instances can include fewer chains such as antibodies naturally occurring in camelids, which can comprise only heavy chains, and V NAR domains from sharks. Antibody products can be derived solely from a single source, or can be “chimeric,” that is, different portions of the antibody can be derived from two different antibodies. For example, the complementarity determining regions, which impart the binding specificity of an antibody, can be derived from a rat or murine source, while the framework portion of the variable regions is derived from a different species source, such as a human. In other chimeric forms, the light and heavy variable domains (optionally with a constant domain) can be derived from one species and one or more constant domains from another species. See, e.g., U.S. patent Ser. No. 11/352,444. The antibody products provided can be produced in hybridomas, by recombinant DNA techniques, or by enzymatic or chemical cleavage of intact antibodies. Unless otherwise indicated, the term “antibody product” includes, in addition to antibodies comprising two full-length heavy chains and two full-length light chains (such as IgG antibodies), antibodies of other isotypes, derivatives, variants, and fragments thereof. The antibody products provided include, but are not limited to, monoclonal antibodies, human antibodies, chimeric antibodies, and humanized antibodies. Immunologically functional antibody fragments provided include, but are not limited to, scFv, Fab, Fab′, F(ab′)₂, and domain antibody products.

An “immunologically functional fragment” (or simply “fragment”) of an immunoglobulin, as used herein, refers to a portion of an antibody, comprising a light chain or a heavy chain (or both) and is capable of binding specifically to an antigen, but the light chain or heavy chain (or both) lacks at least some of the amino acids present in a full-length chain. Such fragments are biologically active in that they bind specifically to the target antigen and can compete with intact antibodies for specific binding to a given epitope. Such a fragment will retain at least one CDR present in the full-length light or heavy chain and can comprise a single heavy chain and/or light chain or portion thereof. These biologically active fragments can be produced by recombinant DNA techniques or can be produced by enzymatic or chemical cleavage of intact antibodies. Immunologically functional immunoglobulin fragments include, but are not limited to, Fab, Fab′, F(ab′)₂, Fv, domain antibodies and single-chain antibodies, and can be derived from any mammalian source, including but not limited to human, mouse, rat, camelid or rabbit. It is contemplated further that a functional portion of the inventive antibodies, for example, one or more CDRs, could be covalently bound to a second protein or to a small molecule to create a therapeutic agent directed to a particular target in the body, possessing bifunctional therapeutic properties, or having a prolonged serum half-life.

A “Fab fragment” comprises one light chain (VL+CL) and a portion of a heavy chain that includes the variable domain and the CH1 domain (VH+CH1). The heavy chain of a Fab molecule cannot form a disulfide bond with another heavy chain molecule.

An “Fc” region contains two heavy chain fragments each comprising the CH2 and CH3 domains of an antibody and in some cases the lower hinge region. The two heavy chain fragments are held together by two or more disulfide bonds (typically in the hinge region) and by hydrophobic interactions of the CH3 domains.

A “Fab′ fragment” contains one light chain and a portion of one heavy chain that contains the VH domain and the CH1 domain and also the region between the CH1 and CH2 domains, such that an interchain disulfide bond can be formed between the two heavy chains of two Fab′ fragments to form a F(ab′)₂ molecule.

A “F(ab′)₂ fragment” contains two light chains and two heavy chains each containing a portion of the constant region between the CH1 and CH2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable domains from both the heavy and light chains, but lacks the constant domains.

“Single-chain antibodies” are Fv molecules in which the heavy and light chain variable domains have been connected by a flexible linker to form a single polypeptide chain, which forms an antigen-binding region. Single chain antibodies are discussed in detail, for example, in PCT Pub. No. WO 88/01649 and U.S. Pat. Nos. 4,946,778 and 5,260,203.

A “domain antibody” is an immunologically functional immunoglobulin fragment containing only the variable domain of a heavy chain or the variable domain of a light chain. In some instances, two or more VH domains are covalently joined with a peptide linker to create a bivalent domain antibody. The two VH domains of a bivalent domain antibody can target the same or different antigens.

A “bivalent antibody” comprises two antigen binding sites. In some instances, the two binding sites have the same antigen specificities. However, bivalent antibodies can be bispecific (see below).

A “multispecific antibody” is one that targets more than one antigen or epitope.

A “bispecific,” “dual-specific” or “bifunctional” antibody is a hybrid antibody having two different antigen binding sites. Bispecific antibodies are a species of multispecific antibody and can be produced by a variety of methods including, but not limited to, fusion of hybridomas or linking of Fab′ fragments. See, e.g., Songsivilai and Lachmann, Clin Exp Immunol. 1990 79:315-21; Kostelny et al., J Immunol. 1992 148:1547-53. The two binding sites of a bispecific antibody will bind to two different epitopes, which can reside on the same or different protein targets. A “trispecific” antibody has three different antigen binding sites. See also, Merchant et al., Nat Biotechnol. 1998 16:677-81.

The term “neutralizing antibody” refers to an antibody that binds to a ligand, prevents binding of the ligand to its binding partner and interrupts the biological response that otherwise would result from the ligand binding to its binding partner. In assessing the binding and specificity of an antibody or immunologically functional fragment thereof, an antibody or fragment will substantially inhibit binding of a ligand to its binding partner when an excess of antibody reduces the quantity of binding partner bound to the ligand by at least about 20%, 30%, 40%, 50%, 60%, 70%, 80%, 85%, 90%, 95%, 97%, 99% or more (as measured in an in vitro competitive binding assay). In the case of antibody products that bind to LILRB1 or LILRB2 proteins, a neutralizing antibody product will diminish the ability of a LILRB1 protein or a LILRB2 protein (or both) to bind to one or more of its (their) ligands thereby inhibiting the LILRB-mediated activity (e.g., as shown in the Examples herein).

The term “competition” when used in the context of antibody products that compete for the same epitope means competition between antibodies is determined by an assay in which the antibody product under test prevents or inhibits specific binding of a reference antibody product to a common antigen (e.g., LILRB2 or LILRB1 or a fragment thereof). Numerous types of competitive binding assays can be used, for example: solid phase direct or indirect radioimmunoassay (RIA), solid phase direct or indirect enzyme immunoassay (EIA), sandwich competition assay [e.g., Stahli et al., Methods Enzymol 1983 9:242-53]; solid phase direct biotin-avidin EIA [e.g., Kirkland et al., J Immunol 1986 137:3614-9]; solid phase direct labeled assay, solid phase direct labeled sandwich assay [e.g., Harlow and Lane, Antibodies, A Laboratory Manual, Cold Spring Harbor Press (1988)]; solid phase direct label RIA using I-125 label [e.g., Morel et al., Molec Immunol. 1988 25:7-15]; solid phase direct biotin-avidin EIA [e.g., Cheung et al., Virology. 1990 176:546-52]; and direct labeled RIA [Moldenhauer et al., Scand J Immunol. 1990 32:77-82]. Typically, such an assay involves the use of purified antigen bound to a solid surface or cells bearing either of these, an unlabeled test antibody and a labeled reference antibody. Competitive inhibition is measured by determining the amount of label bound to the solid surface or cells in the presence of the test antibody. Usually, the test antibody is present in excess. Antibodies identified by competition assay (competing antibodies) include antibodies binding to the same epitope as the reference antibody and antibodies binding to an epitope sufficiently proximal to the epitope bound by the reference antibody for steric hindrance to occur. Usually, when a competing antibody is present in excess, it will inhibit specific binding of a reference antibody to a common antigen by at least 40%, 45%, 50%, 55%, 60%, 65%, 70% or 75%. In some instance, binding is inhibited by at least 80%, 85%, 90%, 95%, or 97% or more by a selective binding agent, such as an antibody, and additionally capable of being used in an animal to produce antibodies capable of binding to that antigen. An antigen can possess one or more epitopes that can interact with different antibodies.

The term “epitope” includes any determinant capable of specifically binding to an antibody or to a T-cell receptor. An epitope is a region of an antigen that is bound by an antibody that specifically targets that antigen, and when the antigen is a protein, includes specific amino acids that directly contact the antibody. Most often, epitopes reside on proteins, but in some instances can reside on other kinds of molecules, such as nucleic acids. Epitope determinants can include chemically active surface groupings of molecules such as amino acids, sugar side chains, phosphoryl or sulfonyl groups, and can have specific three-dimensional structural characteristics, and/or specific charge characteristics. Generally, antibodies specific for a particular target antigen will preferentially recognize an epitope on the target antigen in a complex mixture of proteins and/or macromolecules.

An antibody product “specifically binds” its target antigen when the dissociation constant (K_{d}) is less than 100 nM. The antibody specifically binds antigen with “high affinity” when the K_{d} is less than 10 nM and with “very high affinity” when the K_{d} is less than 0.5 nM. The antibody product can have a K_{d} in the range from about 0.5 nM to about 500 nM. The antibody product can have a K_{d} in the range from about 100 to about 500 nM. One of skill in the art will recognize that specifically binding does not mean exclusive binding, rather it allows for some degree of non-specific binding as is typical in biological reactions between groups with affinity to one another.

As used herein, the term “affinity” refers to the equilibrium constant for the reversible binding of two agents and can be expressed as the equilibrium dissociation constant, K_{D}, a calculated ratio of the dissociation constant and the association constant (K_{off}/Kₒₙ), between the antibody and its antigen. Affinity can also be expressed as the association constant, K_{A}, which is the reciprocal of K_{D}. The antibody products disclosed herein exhibit binding affinity as measured by K_{D} for human LILRB2 and for human LILRB1 in the range of 10⁻⁴ M or less, or ranging down to 10⁻¹⁶ M or lower, (e.g., about 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, 10⁻¹⁵, 10⁻¹⁶ M or less). Antibodies described herein can specifically bind to a human LILRB2 polypeptide or a human LILRB1 polypeptide with a K_{D} of less than or equal to 10⁻⁴ M, less than or equal to about 10⁻⁵ M, less than or equal to about 10⁻⁶ M, less than or equal to 10⁻⁷ M, or less than or equal to 10⁻⁸ M. Methods for determining the affinity of two molecules are well known in the art and include, for example, equilibrium dialysis, surface plasmon resonance (SPR), bio-layer interferometry (BLI), and the like.

As used herein, an antibody product is said to be “immunospecific” or “specific” for, or to “specifically bind” when that an antibody product forms a complex with an antigen that is relatively stable under physiologic conditions. The terms “preferentially binds” or “specifically binds” mean that the antibodies or fragments thereof bind to an epitope with greater affinity than it binds unrelated amino acid sequences, and, if cross-reactive to other polypeptides containing the epitope, are not toxic at the levels at which they are formulated for use in administration to human subjects. The terms are also applicable where for example, an antibody product is specific for a particular epitope that is carried by more than one antigen, in which case the antibody or antigen-binding fragment thereof carrying the antigen-binding domain will be able to specifically bind to the epitope found in the different antigens. The measured relative affinity of antibodies for one epitope compared to another may differ between comparators, or between variants of the same comparator, or across experimental conditions. The LILRB2/1 antibodies disclosed herein preferentially bind LILRB1 and LILRB2 as compared to other LILRA and LILRB family members. Such relative affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, at least 200-fold greater, at least 250-fold greater, at least 500-fold greater, or at least 1000-fold greater than the affinity of the antibody product for amino acid sequences derived from other LILR family members.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) must be addressed by a particular mathematical model or computer program (i.e., an “algorithm”). Methods that can be used to calculate the identity of the aligned nucleic acids or polypeptides include those described in Computational Molecular Biology, (Lesk, Ed.), 1988, New York: Oxford University Press; Biocomputing Informatics and Genome Projects, (Smith, ed.), 1993, New York: Academic Press; Computer Analysis of Sequence Data, Part I, (Griffin and Griffin, eds.), 1994, New Jersey: Humana Press; Sequence Analysis in Molecular Biology, (von Heinje), 1987, New York: Academic Press; Sequence Analysis Primer, (Gribskov and Devereux, eds.), 1991, New York: M. Stockton Press; and Carillo et al., SIAM J Applied Math. 1988 48(5):1073-82.

In calculating percent identity, the sequences being compared are aligned in a way that gives the largest match between the sequences. An exemplary computer program used to determine percent identity is the GCG program package, which includes GAP (Devereux et al., Nucl Acid Res. 1984 12:387-95; Genetics Computer Group, University of Wisconsin, Madison, Wisc.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3 times (3×) the average diagonal, in which the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix [e.g., Dayhoff et al., Atlas of Protein Sequence and Structure, 5:345-352 (1978) for the PAM 250 comparison matrix; Henikoff et al., Proc Natl Acad Sci USA. 1992 89:10915-9 for the BLOSUM 62 comparison matrix] can also be used by the algorithm.

Recommended parameters for determining percent identity for polypeptides or nucleotide sequences using the GAP program are the following: Algorithm: Needleman et al., J Mol Biol. 1970 48:443-53; Comparison matrix: BLOSUM 62 from Henikoff et al., 1992, supra; Gap Penalty: 12 (but with no penalty for end gaps); Gap Length Penalty: 4; Threshold of Similarity: 0.

Certain alignment schemes for aligning two amino acid sequences can result in matching of only a short region of the two sequences, and this small aligned region can have very high sequence identity even though there is no significant relationship between the two full-length sequences. Accordingly, the selected alignment method (GAP program) can be adjusted if so desired to result in an alignment that spans at least 50 contiguous amino acids of the target polypeptide.

Other exemplary programs that compare and align pairs of sequences include, but are not limited to, ALIGN (Myers and Miller, Comput Appl Biosci. 1988 4(1):11-7); FASTA (Pearson and Lipman, Proc Natl Acad Sci USA. 1988 85(8):2444-8; Pearson, Methods Enzymol. 1990 183:63-98); and gapped BLAST (Altschul et al., Nucleic Acids Res 1997 25(17):3389-402), BLASTP, BLASTN, or GCG (Devereux et al., Nucleic Acids Res. 1984 12(1 Pt 1):387-95)).

“Amino acid” includes its normal meaning in the art. The twenty naturally occurring amino acids and their abbreviations follow conventional usage. See Immunology—A Synthesis, 2nd ed. (Golub and Gren, Eds.), Sinauer Associates: Sunderland, Mass. (1991). Stereoisomers (e.g., D-amino acids) of the twenty conventional amino acids, unnatural amino acids such as α-, α-disubstituted amino acids, N-alkyl amino acids, and other unconventional amino acids can be suitable components. Examples of unconventional amino acids include: 4-hydroxyproline, gamma-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxyl-terminal direction, in accordance with standard usage and convention.

Table 1 below sets out heavy and light chain variable domains for an LILRB-specific antibody designated B21A, and corresponding full-length heavy (hIgG1 and hIgG4) and light (kappa) chains provided herein. CDRs of the heavy chain variable (VH) domain (SEQ ID NO: 1) and the light chain variable (VL) domain (SEQ ID NO: 2) are indicated: IMGT in double underline; Kabat in bold. The heavy (H) and light (L) chain IMGT and Kabat CDRs are presented in Table 2.

TABLE 1
Variable Domains and Full Length Kappa Light and IgG1 and IgG4 Heavy Chains

| SEQ ID NO: | Name | Sequence |
|---|---|---|
| 1 | B21A-VH | QSVEESGGRLVTPGTPLTLTCTVSGFSLNTYAMTWVRQAPGKGLEWIGIIVASGGTYY |
| | | ASWAKGRFTISRTSTTVVMKITSPTTEDTATYFCARGGMGSDWGESLDAFDPWGQGTL |
| | | VTVSS |
| 2 | B21A-VL | DIVMTQTPASVEAAVGGTVTINCQASGNIYSFLAWYQQKPGQPPKLLIYTASNLETGV |
| | | PSRFSGSGSGTEYTLTISGVQCDDAATYYCLGGLYSSDVSYAFGGGTEVVVK |
| 3 | B21A-HC1 | QSVEESGGRLVTPGTPLTLTCTVSGFSLNTYAMTWVRQAPGKGLEWIGIIVASGGTYY |
| | (IgG1) | ASWAKGRFTISRTSTTVVMKITSPTTEDTATYFCARGGMGSDWGESLDAFDPWGQGTL |
| | | VTVSSASTKGPSVFPLAPSSKSTSGGTAALGCLVKDYFPEPVTVSWNSGALTSGVHTF |
| | | PAVLQSSGLYSLSSVVTVPSSSLGTQTYICNVNHKPSNTKVDKRVEPKSCDKTHTCPP |
| | | CPAPELLGGPSVFLFPPKPKDTLMISRTPEVTCVVVDVSHEDPEVKFNWYVDGVEVHN |
| | | AKTKPREEQYNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKTTPPVLDSDG |
| | | SFFLYSKLTVDKSRWQQGNVFSCSVMHEALHNHYTQKSLSLSPGK |
| 4 | B21A-HC4 | QSVEESGGRLVTPGTPLTLTCTVSGFSLNTYAMTWVRQAPGKGLEWIGIIVASGGTYY |
| | (IgG4) | ASWAKGRFTISRTSTTVVMKITSPTTEDTATYFCARGGMGSDWGESLDAFDPWGQGTL |
| | | VTVSSASTKGPSVFPLAPCSRSTSESTAALGCLVKDYFPEPVTVSWNSGALTSGVHTF |
| | | PAVLQSSGLYSLSSVVTVPSSSLGTKTYTCNVDHKPSNTKVDKRVESKYGPPCPPCPA |
| | | PEFLGGPSVFLFPPKPKDTLMISRTPEVTCVVVDVSQEDPEVQFNWYVDGVEVHNAKT |
| | | KPREEQFNSTYRVVSVLTVLHQDWLNGKEYKCKVSNKGLPSSIEKTISKAKGQPREPQ |
| | | VYTLPPSQEEMTKNQVSLTCLVKGFYPSDIAVEWESNGQPENNYKTTPPVLDSDGSFF |
| | | LYSRLTVDKSRWQEGNVFSCSVMHEALHNHYTQKSLSLSLGK |
| 5 | B21A-LC | DIVMTQTPASVEAAVGGTVTINCQASGNIYSFLAWYQQKPGQPPKLLIYTASNLETGV |
| | (Kappa) | PSRFSGSGSGTEYTLTISGVQCDDAATYYCLGGLYSSDVSYAFGGGTEVVVKRTVAAP |
| | | SVFIFPPSDEQLKSGTASVVCLLNNFYPREAKVQWKVDNALQSGNSQESVTEQDSKDS |
| | | TYSLSSTLTLSKADYEKHKVYACEVTHQGLSSPVTKSFNRGEC |

TABLE 2
Complementarity Determining Regions

| SEQ ID NO: | Description | Sequence |
|---|---|---|
| 16 | B21A CDR-H1 IMGT | GFSLNTYA |
| 17 | B21A CDR-H2 IMGT | IVASGGT |
| 18 | B21A CDR-H3 IMGT | ARGGMGSDWGESLDAFDP |
| 19 | B21A CDR-L1 IMGT | GNIYSF |
| 20 | B21A CDR-L2 IMGT | TAS |
| 21 | B21A CDR-L3 IMGT | LGGLYSSDVSYA |
| 22 | B21A CDR-H1 Kabat | TYAMT |
| 23 | B21A CDR-H2 Kabat | IIVASGGTYYASWAKG |
| 24 | B21A CDR-H3 Kabat | GGMGSDWGESLDAFDP |
| 25 | B21A CDR-L1 Kabat | QASGNIYSFLA |
| 26 | B21A CDR-L2 Kabat | TASNLET |
| 27 | B21A CDR-L3 Kabat | LGGLYSSDVSYA |

Humanized variants of the B21A antibody were prepared as described in the Examples. Variable domains of representative humanized heavy and light chains are presented in Table 3.

TABLE 3
Humanized Variable Domains

| SEQ ID NO: | Name | Sequence |
|---|---|---|
| 6 | B21H-VH1 | QVQLVESGGGVVQPGTSLRLSCAASGFSLNTYAMTWVRQAPGKGLEWVAIIVASGG |
| | | TYYASWAKGRFTISRDTSKNTVVMQITSLRAEDTAVYFCARGGMGSDWGESLDAFD |
| | | PWGQGTLVTVSS |
| 7 | B21H-VH2 | EVQLLESGGGLVQPGGSLRLSCAASGFSLNTYAMTWVRQAPGKGLEWVSIIVASGG |
| | | TYYASWAKGRFTISRDNSKNTLYLQMNSLTVEDTAVYFCARGGMGSDWGESLDAFD |
| | | PWGQGTLVTVSS |
| 8 | B21H-VH3 | EVQLVESGGDLAQPGGSLRLSCAVSGFSLNTYAMTWVRQAPGKGLEWVSIIVASGG |
| | | TYYASWAKGRFTISRDNSKNTVYLQMTSLRAEDTALYFCARGGMGSDWGESLDAFD |
| | | PWGQGTLVTVSS |
| 9 | B21H-VH4 | EVQLVESGGGLVQPGGSLRLSCAASGFSLNTYAMTWVRQAPGKGLEWISIIVASGG |
| | | TYYASWAKGRFTISRDNAKSSVYLQLTSLTTEDTAVYYCARGGMGSDWGESLDAFD |
| | | PWGQGTMVTVSS |
| 10 | B21H-VH5 | QVQLEESGGRLVQPGTSLRLSCAVSGFSLNTYAMTWVRQAPGKGLEWIGIIVASGG |
| | | TYYASWAKGRFTISRDTSKNTLYLQITSLRAEDTATYFCARGGMGSDWGESLDAFD |
| | | PWGQGTLVTVSS |
| 11 | B21H-VL1 | DIQMTQSPSTLSASVGDRVTITCQASGNIYSFLAWYQQKPGKAPKLLIYTASNLET |
| | | GVPSRFSGSGSGTEYTLTISSLQPDDFATYYCLGGLYSSDVSYAFGPGTKVEIK |
| 12 | B21H-VL2 | DIQMTQSPSSLSASVGDRVTITCQASGNIYSFLAWYQQKPGKVPKLLIYTASNLET |
| | | GVPSRFSGSGSGTDYTLTISSLQSEDAATYYCLGGLYSSDVSYAFGGGTKVVIK |
| 13 | B21H-VL3 | DIVMTQSPSSVSASVGDRVTITCQASGNIYSFLAWYQQKPGKAPKLLIYTASNLET |
| | | GVPSRFSGSGSGTDFTLTISSLQPEDFATYYCLGGLYSSDVSYAFGGGTKVVIK |
| 14 | B21H-VL4 | DVVMTQSPSTVSASVGDRVTLTCQASGNIYSFLAWYQQKPGQPPKLLIYTASNLET |
| | | GVPDRFSGSGSGTDYTLTISSLQADDFATYYCLGGLYSSDVSYAFGQGTKVEIK |
| 15 | B21H-VL5 | DIQMTQTPSTVEASVGGTVTITCQASGNIYSFLAWYQQKPGQPPKLLIYTASNLET |
| | | GVPSRFSGSGSGTEYTLTISGLQSDDAATYYCLGGLYSSDVSYAFGGGTEVVVK |

The skilled artisan recognizes that antibody products, such as full-length intact LILRB2/1 antibodies and LILRB2- and LILRB1-binding antibody fragments, can be prepared based the heavy chain and light chain variable domains given in Tables 1 and 3, or on the CDRs given in Table 2. To demonstrate, as described in the Examples, in addition to the full-length antibody chains shown in Table 1, full-length IgG1 or IgG4 heavy chains were prepared comprising the VH variable domains shown in Table 3 (SEQ ID NOs: 6-10), in combination with kappa light chains comprising the VL domains shown in Table 3 (SEQ ID NOs: 11-15). Full length IgG4 heavy chain sequences are presented as SEQ ID NOS: 28-32. Full length IgG1 heavy chain sequences are presented as SEQ ID NOS: 33-37. Full length kappa light chains sequences are presented as SEQ ID NOS: 38-42. LILRB2/1-binding antibody products were prepared using all combinations of the heavy and light chains, and the testing of some of these in various non-clinical assays is described in the Examples.

Antibody products can comprise a light chain variable domain comprising a sequence of amino acids that differs from the sequence of a light chain variable domain described herein at only 1, 2, 3, 4, 5, 6, 7, 8, 9, 10, 11, 12, 13, 14, or 15 amino acid residues, in which each such sequence difference is independently either a deletion, insertion or substitution of one amino acid. The light chain variable region in some antibodies comprises a sequence of amino acids that has at least 70%, 75%, 80%, 85%, 90%, 95%, 96%, 97%, 98%, or 99% sequence identity to an amino acid sequence of a light chain variable region in Table 1 or in Table 3.

Antibody products can comprise a heavy chain variable domain comprising a sequence of amino acids that differs from the sequence of a heavy chain variable domain provided herein at only 1, 2, 3, 4, 5, 6, 7, 8, 9, 10, 11, 12, 13, 14 or 15 amino acid residues, in which each such sequence difference is independently either a deletion, insertion or substitution of one amino acid. The heavy chain variable region in some antibodies comprises a sequence of amino acids that has at least 70%, 75%, 80%, 85%, 90%, 95%, 96%, 97%, 98% or 99% sequence identity to the amino acid sequence of the heavy chain variable region in Table 1 or in Table 3 Still other antibody products include variant forms of a variant light chain and a variant heavy chain as just described.

The antibody products that are provided can include one, two, three, four, five or all six CDRs. Some antibody products include both the light chain CDR3 and the heavy chain CDR3. Certain antibody products have variant forms of the CDRs, with one or more (i.e., 2, 3, 4, 5 or 6) of the CDRs each having at least 80%, 85%, 90%, 95%, 96%, 97%, 98% or 99% sequence identity to a CDR sequence. For example, the antibody product can include both a light chain CDR3 and a heavy chain CDR3 that each have at least 80%, 85%, 90%, 95%, 96%, 97%, 98% or 99% sequence identity to the light chain CDR3 sequence and the heavy chain CDR3, respectively. The CDR sequences of the antibody products that are provided can also differ from the CDR sequences in Table 11 or in Table 3 such that the amino acid sequence for any given CDR differs from the sequence listed in Table 1 or in Table 3 by no more than one, two, three, four or five amino acid residues. Differences from the listed sequences are conservative substitutions.

When an antibody product is said to bind an epitope within LILRB2, what is meant is that the antibody product specifically binds to a polypeptide consisting of the specified residues (e.g., a specified segment of LILRB2). Such an antibody does not necessarily contact every residue within LILRB2. Nor does every single amino acid substitution or deletion within LILRB2 necessarily significantly affect binding affinity. Exact epitope specificity of an antibody can be determined in variety of ways. One approach, for example, involves testing a collection of overlapping peptides of about fifteen amino acids spanning the sequence of LILRB2 and differing in increments of a small number of amino acids (e.g., three amino acids). The peptides are immobilized within the wells of a microtiter dish. Immobilization can be achieved by biotinylating one terminus of the peptides. Optionally, different samples of the same peptide can be biotinylated at the N and C terminus and immobilized in separate wells for purposes of comparison. This is useful for identifying end-specific antibodies. Optionally, additional peptides can be included terminating at a particular amino acid of interest. This approach is useful for identifying end-specific antibodies to internal fragments of LILRB2. An antibody product is screened for specific binding to each of the various peptides. The epitope is defined as occurring within a segment of amino acids that is common to all peptides to which the antibody shows specific binding.

Antibody products that compete with one of the exemplified antibodies for specific binding to LILRB2 are also provided. Such antibody products can also bind to the same epitope as one of the exemplified antibodies. Antibody products that compete with or bind to the same epitope as the exemplified antibody or fragment are expected to show similar functional properties. The exemplified antibody products include those with the heavy and light chains, variable domains and CDRs provided in Tables 1, 2, or 3. Competing antibody products can include those that bind to the epitope described in the section on antibodies and epitopes above.

The antibody products provided include monoclonal antibodies that bind to LILRB2. Monoclonal antibodies can be produced using any technique known in the art, e.g., by immortalizing spleen cells harvested from the transgenic animal after completion of the immunization schedule. The spleen cells can be immortalized using any technique known in the art, e.g., by fusing them with myeloma cells to produce hybridomas. Myeloma cells for use in hybridoma-producing fusion procedures preferably are non-antibody-producing, have high fusion efficiency, and enzyme deficiencies that render them incapable of growing in certain selective media that support the growth of only the desired fused cells (hybridomas). Examples of suitable cell lines for use in mouse fusions include Sp-20, P3-X63/Ag8, P3-X63-Ag8.653, NS1/1.Ag 4 1, Sp210-Ag14, FO, NSO/U, MPC-11, MPC11-X45-GTG 1.7. and S194/5XXO Bul; examples of cell lines used in rat fusions include R210.RCY3, Y3-Ag 1.2.3, IR983F, and 4B210. Other cell lines useful for cell fusions are U-266, GM1500-GRG2, LICR-LON-HMy2, and UC729-6.

In some instances, a hybridoma cell line is produced by immunizing an animal (e.g., a transgenic animal having human immunoglobulin sequences) with a LILRB2 immunogen; harvesting spleen cells from the immunized animal; fusing the harvested spleen cells to a myeloma cell line, thereby generating hybridoma cells; establishing hybridoma cell lines from the hybridoma cells, and identifying a hybridoma cell line that produces an antibody that binds a LILRB2 polypeptide. Hybridoma cell lines, and LILRB2 monoclonal antibodies produced by them, are provided herein.

Monoclonal antibodies secreted by a hybridoma cell line can be purified using any useful technique known in the antibody arts. Hybridomas or monoclonal antibodies can be further screened to identify monoclonal antibodies with particular properties. Examples of such screens are provided in the Examples below.

Chimeric and humanized antibodies based upon the foregoing sequences are also provided. Monoclonal antibodies for use as therapeutic agents can be modified in various ways prior to use. One example is a “chimeric” antibody, which is an antibody composed of protein segments from different antibodies that are covalently joined to produce functional immunoglobulin light or heavy chains or immunologically functional portions thereof. Generally, a portion of the heavy chain and/or light chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. For methods relating to chimeric antibodies, see, for example, U.S. Pat. No. 4,816,567; and Morrison et al., Proc Natl Acad Sci USA. 1985 81:6851-5. CDR grafting is described, for example, in U.S. Pat. Nos. 6,180,370, 5,693,762, 5,693,761, 5,585,089, and 5,530,101.

Generally, the goal of making a chimeric antibody is to create a chimera in which the number of amino acids from the intended patient species is maximized. One example is the “CDR-grafted” antibody, in which the antibody comprises one or more complementarity determining regions (CDRs) from a particular species or belonging to a particular antibody class or subclass, while the remainder of the antibody chain(s) is/are identical with or homologous to a corresponding sequence in antibodies derived from another species or belonging to another antibody class or subclass. For use in humans, the V region or selected CDRs from a rodent antibody often are grafted into a human antibody, replacing the naturally occurring V regions or CDRs of the human antibody.

“Humanized” antibody products are provided. Generally, a humanized antibody is produced from a monoclonal antibody raised initially in a non-human animal. Certain amino acid residues in this monoclonal antibody, typically from non-antigen recognizing portions of the antibody, are modified to be homologous to corresponding residues in a human antibody of corresponding isotype. Humanization can be performed, for example, using various methods by substituting at least a portion of a rodent variable region with the corresponding regions of a human antibody [e.g., U.S. Pat. Nos. 5,585,089, and 5,693,762; Jones et al., Nature. 1986 321:522-5; Riechmann et al., Nature. 1988 332:323-7; Verhoeyen et al., Science. 1988 239:1534-6]. Constant regions from species other than human can be used along with the human variable region(s) to produce hybrid antibodies.

Fully human antibodies are also provided. Methods are known for making fully human antibodies specific for a given antigen without exposing human beings to the antigen (“fully human antibodies”). One means for implementing the production of fully human antibodies is the “humanization” of the mouse humoral immune system. Introduction of human immunoglobulin (Ig) loci into mice in which the endogenous Ig genes have been inactivated is one means of producing fully human monoclonal antibodies (MAbs) in mouse, an animal that can be immunized with any desirable antigen. Using fully human antibodies can minimize the immunogenic and allergic responses that can sometimes be caused by administering mouse or mouse-derivatized monoclonal antibodies to humans as therapeutic agents.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, for example, Jakobovits et al., Proc Natl Acad Sci USA. 1993 90:2551-5; Jakobovits et al., Nature. 1993 362:255-8; and Bruggemann et al., Year Immunol. 1993 7:33-40. In one example of such a method, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then cross-bred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, WO96/33735 and WO94/02602. Additional methods relating to transgenic mice for making human antibodies are described in U.S. Pat. Nos. 5,545,807; 6,713,610; 6,673,986; 6,162,963; 5,545,807; 6,300,129; 6,255,458; 5,877,397; 5,874,299 and 5,545,806; in PCT Pub. Nos. WO 91/10741, WO 90/04036, and in EPO Pub. No. EP 546073B1. The transgenic mice, referred to herein as “HuMab” mice, contain a human immunoglobulin gene minilocus that encodes unrearranged human heavy (g and gamma) and kappa light chain immunoglobulin sequences, together with targeted mutations that inactivate the endogenous gamma and kappa chain loci (Lonberg et al., Nature. 1994 368:856-9). Accordingly, the aforementioned mice exhibit reduced expression of mouse IgM or kappa and in response to immunization the introduced human heavy and light chain transgenes undergo class switching and somatic mutation to generate high affinity human IgG kappa monoclonal antibodies. An exemplary mouse with the entire human immunoglobulin locus in its germline is the XenoMouse (Abgenix). Another is the VelociImmune mouse (Regeneron Pharmaceuticals). Others are the RenMab mouse and the RenLite mouse (Biocytogen), and, more recently the AlivaMab mouse (Ablexis) and ATX-GX mouse (Alloy Therapeutics).

Using hybridoma technology, antigen-specific human monoclonal antibodies with the desired specificity can be produced and selected from the transgenic mice such as those described above. Such antibodies can be cloned and expressed using a suitable vector and host cell, or the antibodies can be harvested from cultured hybridoma cells.

Fully human antibodies can also be derived from phage-display libraries (as disclosed in Hoogenboom and Winter, J Mol Biol. 1992 227(2):381-8; and Marks et al., J Mol Biol. 1991 222:581-97. Phage display techniques mimic immune selection through the display of antibody repertoires on the surface of filamentous bacteriophage, and subsequent selection of phage by their binding to an antigen of choice. One such technique is described in PCT Pub. No. WO 99/10494, which describes the isolation of high affinity and functional agonistic antibodies for MPL- and msk-receptors using such an approach.

Single chain antibodies are provided. Single chain antibodies are formed by linking heavy and light chain variable domain (Fv region) fragments (such as those shown in Table 1 or in Table 3) via an amino acid bridge (short peptide linker), resulting in a single polypeptide chain. Such single-chain Fvs (scFvs) can be prepared by fusing DNA encoding a peptide linker between DNAs encoding the two variable domain polypeptides (VL and VH). The resulting polypeptides can fold back on themselves to form antigen-binding monomers, or they can form multimers (e.g., dimers, trimers, or tetramers), depending on the length of a flexible linker between the two variable domains. Techniques developed for the production of single chain antibodies include those described in U.S. Pat. No. 4,946,778; Bird et al., Science. 1988 242:423-6; Huston et al., Proc Natl Acad Sci USA. 1988 85:5879-83; Ward et al., Nature. 1989 334:544-6; and de Graaf et al., Methods Mol Biol. 2002 178:379-87. A “diabody” is a dimer of scFV.

Antibodies provided herein that are of one subclass can be changed to antibodies from a different subclass using subclass switching methods. For example, the variable domains depicted in Table 1 or in Table 3 can be attached to constant domains of any desired Ig subtype. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques can be employed. Cloned DNA encoding particular antibody polypeptides can be employed in such procedures, e.g., DNA encoding the constant domain of an antibody of the desired isotype. See, e.g., Lantto et al., Methods Mol Biol. 2002 178:303-16. Accordingly, the antibodies that are provided include a desired isotype (for example, IgA, IgG1, IgG2, IgG3, IgG4, IgE, and IgD).

Antibody products provided can include one or more of the CDRs of any of the heavy chain variable domains exemplified herein, where such CDRs are determined according to IMGT, Kabat or other method: (i) a CDR-H1 with at least 80% sequence identity to the CDR-H1 of SEQ ID NO: 1, 6, 7, 8, 9, or 10; (ii) a CDR-H2 with at least 80% sequence identity to the CDR-H2 of SEQ ID NO: SEQ ID NO: 1, 6, 7, 8, 9, or 10; and (iii) a CDR-H3 with at least 80% sequence identity to the CDR-H3 of SEQ ID NO: SEQ ID NO: 1, 6, 7, 8, 9, or 10. Antibody products provided can include one or more of the CDRs of any of the light chain variable domains exemplified herein, where such CDRs are determined according to IMGT, Kabat or other method: (i) a CDR-L1 with at least 80% sequence identity to the CDR-L1 of SEQ ID NO: 2, 11, 12, 13, 14, or 15; (ii) a CDR-L2 with at least 80% sequence identity to the CDR-L2 of SEQ ID NO: SEQ ID NO: 2, 11, 12, 13, 14, or 15; and (iii) a CDR-L3 with at least 80% sequence identity to the CDR-L3 of SEQ ID NO: SEQ ID NO: 2, 11, 12, 13, 14, or 15. In some embodiments, the CDRs will have at least 85%, at least 90%, at least 95%, or least 99% identity to the determined CDR sequences. Antibody products can include one, two, three, four, five or all six of the foregoing CDRs, as long as they specifically bind hLILRB2.

Antibody products provided can include one or more of the following exemplary heavy chain IMGT CDRs: (i) a CDR-H1 with at least 80% sequence identity to SEQ ID NO: 16; (ii) a CDR-H2 with at least 80% sequence identity to SEQ ID NO: 17; and (iii) a CDR-H3 with at least 80% sequence identity to SEQ ID NO: 18. Antibody products provided can include one or more of the following light chain CDRs: (i) a CDR-L1 with at least 80% sequence identity to SEQ ID NO: 19; (ii) a CDR-L2 with at least 80% sequence identity to SEQ ID NO: 20; and (iii) a CDR-L3 with at least 80% sequence identity to SEQ ID NO: 21. In some embodiments, the CDRs will have at least 85%, at least 90%, at least 95%, or least 99% identity to the specified CDR sequences. Antibody products can include one, two, three, four, five or all six of the foregoing CDRs, as long as they specifically bind hLILRB2.

Antibody products provided can include one or more of the following exemplary heavy chain Kabat CDRs: (i) a CDR-H1 with at least 80% sequence identity to SEQ ID NO: 22; (ii) a CDR-H2 with at least 80% sequence identity to SEQ ID NO: 23; and (iii) a CDR-H3 with at least 80% sequence identity to SEQ ID NO: 24. Antibody products provided can include one or more of the following light chain CDRs: (i) a CDR-L1 with at least 80% sequence identity to SEQ ID NO: 25; (ii) a CDR-L2 with at least 80% sequence identity to SEQ ID NO: 26; and (iii) a CDR-L3 with at least 80% sequence identity to SEQ ID NO: 27. In some embodiments, the CDRs will have at least 85%, at least 90%, at least 95%, or least 99% identity to the specified CDR sequences. Antibody products can include one, two, three, four, five or all six of the foregoing CDRs, as long as they specifically bind hLILRB2.

Antibody products provided can include (a) a heavy chain variable region having 80%, 85%, 90%, 92%, 95%, 96%, 97%, 98%, 99% or greater sequence identity with SEQ ID NO: 1, 6, 7, 8, 9, or 10; (b) a light chain variable region having at least 80%, 85%, 90%, 92%, 95%, 96%, 97%, 98%, 99% or greater sequence identity with SEQ ID NOs: 2, 11, 12, 13, 14, or 15; or (c) a heavy chain variable region of (a) and a light chain variable region of (b).

Other antibody products provided compete with an antibody such as those described above for specific binding to a LILRB2 polypeptide. For example, antibody products are provided that compete with an antibody that consists of two identical heavy chains and two identical light chains, in which the heavy chains comprise SEQ ID NO: 1, 6, 7, 8, 9, or 10, and the light chains comprise SEQ ID NO: 2, 11, 12, 13, 14, or 15.

LILRB2/1 antibody products are provided that have a half-life of at least one day in vitro or in vivo (e.g., when administered to a human subject). The antibody product can have a half-life of at least three days. The antibody product can have a half-life of four days or longer. The antibody product can have a half-life of eight days or longer.

### Variants

Variant forms of LILRB2/1 antibody products disclosed herein (e.g., variant forms of antibody products having sequences listed in Tables 10 and 12) are provided. For example, antibody products can have one or more conservative amino acid substitutions in one or more of the heavy or light chain variable regions, or CDRs, listed in Tables 10 and 12.

Naturally occurring amino acids can be divided into classes based on common side chain properties: 1) hydrophobic: norleucine, Met, Ala, Val, Leu, Ile; 2) neutral hydrophilic: Cys, Ser, Thr, Asn, Gln; 3) acidic: Asp, Glu; 4) basic: His, Lys, Arg; 5) residues that influence chain orientation: Gly, Pro; and 6) aromatic: Trp, Tyr, Phe. Conservative amino acid substitutions can involve exchange of a member of one of these classes with another member of the same class. Conservative amino acid substitutions can encompass non-naturally occurring amino acid residues, which are typically incorporated by chemical peptide synthesis rather than by synthesis in biological systems. These include peptidomimetics and other reversed or inverted forms of amino acid moieties.

Non-conservative substitutions can involve the exchange of a member of one of the above classes for a member from another class. Such substituted residues can be introduced into regions of the antibody product that are homologous with human antibodies, or into the non-homologous regions of the molecule.

In making such changes, the hydropathic index of amino acids can be considered. The hydropathic profile of a protein is calculated by assigning each amino acid a numerical value (“hydropathy index”) and then repetitively averaging these values along the peptide chain. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic profile in conferring interactive biological function on a protein is understood in the art [e.g., Kyte and Doolittle, J Mol Biol, 157(1):105-132 (1982)]. It is known that certain amino acids can be substituted for other amino acids having a similar hydropathic index or score and still retain a similar biological activity. In making changes based upon the hydropathic index, the substitution of amino acids whose hydropathic indices are within ±0.2 can be made. The substitution of amino acids whose hydropathic indices are within ±0.1 can also be made. The substitution of amino acids whose hydropathic indices are within ±0.5 can also be made.

It is also understood in the art that the substitution of like residues in amino acid sequences can be made effectively on the basis of relative hydrophilicity or hydrophobicity of the residues, particularly where the biologically functional protein or peptide thereby created is intended for use in immunological molecules, as in the present case. The greatest local average hydropathic character of a protein, as governed by the hydrophilicity of its adjacent amino acids, can correlate with its immunogenicity and antigen-binding or immunogenicity, that is, with a biological property of the protein.

Various methods are known for estimating the hydrophilicity or hydrophobicity of amino acid residues in proteins. A comparative survey of such methods is given at Biswas et al., J Chromatogr A. 1000(1-2):637-55. Hopp and Woods (Mol Immunol. 1983 20(4):483-9) assigned the hydrophilicity values amino acid residues: arginine (+3.0); lysine (+3.0); aspartate (+3.0±0.1); glutamate (+3.0±0.1); serine (+0.3); asparagine (+0.2); glutamine (+0.2); glycine (0); threonine (−0.4); proline (−0.5±0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In this ranking system, more hydrophilic residues are assigned positive values, and less hydrophilic residues negative. In making changes based upon similar hydrophilicity values, the substitution of amino acids whose hydrophilicity values are within ±0.2 is included, otherwise those which are within ±0.1 are included or those within ±0.5 are included. In some instances, one can also identify epitopes from primary amino acid sequences on the basis of hydrophilicity. These regions are also referred to as “epitopic core regions.”

A skilled artisan will be able to determine suitable variants of polypeptides as set forth herein using well-known techniques. One skilled in the art can identify suitable areas of the molecule that can be changed without destroying activity by targeting regions not believed to be important for activity. The skilled artisan also will be able to identify residues and portions of the molecules that are conserved among similar polypeptides. Even areas that can be important for biological activity or for structure can be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a protein that correspond to amino acid residues important for activity or structure in similar proteins. One skilled in the art can opt for chemically similar amino acid substitutions for such predicted important amino acid residues.

One skilled in the art can also analyze the three-dimensional structure and amino acid sequence in relation to that structure in similar polypeptides. In view of such information, one skilled in the art can predict the alignment of amino acid residues of an antibody with respect to its three-dimensional structure. One skilled in the art can choose not to make radical changes to amino acid residues predicted to be on the surface of the protein, since such residues can be involved in important interactions with other molecules. Moreover, one skilled in the art can generate test variants containing a single amino acid substitution at each desired amino acid residue. These variants can then be screened using assays for LILRB1 and/or LILRB2 binding activity (see Examples below), thus yielding information regarding which amino acids can be changed and which must not be changed. In other words, based on information gathered from such routine experiments, one skilled in the art can readily determine the amino acid positions where further substitutions should be avoided either alone or in combination with other mutations.

Substantial modifications in the functional and/or biochemical characteristics of the antibody products described herein can be achieved by creating substitutions in the amino acid sequence of the heavy and light chains that differ significantly in their effect on maintaining (a) the structure of the molecular backbone in the area of the substitution, for example, as a sheet or helical conformation, (b) the charge or hydrophobicity of the molecule at the target site, or (c) the bulkiness of the side chain. A “conservative amino acid substitution” can involve a substitution of a native amino acid residue with a normative residue that has little or no effect on the polarity or charge of the amino acid residue at that position. Furthermore, any native residue in the polypeptide can also be substituted with alanine, as has been previously described for alanine scanning mutagenesis.

Amino acid substitutions (whether conservative or non-conservative) of the subject antibodies can be implemented by those skilled in the art by applying routine techniques. Amino acid substitutions include, but are not limited to, substitutions that: (1) reduce susceptibility to proteolysis, (2) reduce susceptibility to oxidation, (3) alter binding affinity for forming protein complexes, (4) alter ligand or antigen binding affinities, and/or (4) confer or modify other physicochemical or functional properties on such polypeptides. For example, single or multiple amino acid substitutions (e.g., conservative amino acid substitutions) can be made in the naturally occurring sequence. Substitutions can be made in that portion of the antibody that lies outside the domain(s) forming intermolecular contacts). Conservative amino acid substitutions can be used that do not substantially change the structural characteristics of the parent sequence (e.g., one or more replacement amino acids that do not disrupt the secondary structure that characterizes the parent or native antibody). Examples of art-recognized polypeptide secondary and tertiary structures are described in Proteins, Structures and Molecular Principles (Creighton, ed.), 1984, New York: W. H. Freeman and Company; Introduction to Protein Structure (Branden and Tooze, eds.), 1991, New York: Garland Publishing; and Thornton et al., Nature. 1991 354(6349):105, which are each incorporated herein by reference.

Glycosylation variants of the antibody products are provided in which the number and/or type of glycosylation site(s) has been altered compared to the amino acid sequences of the parent polypeptide. Antibody product variants can comprise a greater or a lesser number of N-linked glycosylation sites than the native antibody. An N-linked glycosylation site is characterized by the sequence: Asn-X-Ser or Asn-X-Thr, in which the amino acid residue designated as X can be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. For example, one or more new N-linked sites are created. Antibodies typically have a N-linked glycosylation site in the Fc region.

Additional antibody product variants include cysteine variants in which one or more cysteine residues in the parent or native amino acid sequence are deleted from or substituted with another amino acid (e.g., serine). Cysteine variants are useful, inter alia when antibodies must be refolded into a biologically active conformation. Cysteine variants can have fewer cysteine residues than the native antibody, and typically have an even number to minimize interactions resulting from unpaired cysteines.

### Effector Functions

Antibody structure affects the role that the antibody plays in the immune system and the effects that the antibody can induce or influence. See, e.g., Vidarsson et al., Front Immunol. 2014 5(Art. 5):1-17. Examples of antibody effector functions include: C1q binding and complement dependent cytotoxicity; Fc receptor binding; antibody-dependent cell-mediated cytotoxicity (ADCC); phagocytosis; down-regulation of cell surface receptors (e.g., B cell receptor); and B cell activation. Typically, the Fc-mediated functions involve binding of the Fc portion of an antibody by specialized receptor molecules, “Fc receptors” or “FcR,” expressed by the cell whose function is to be affected.

IgG is considered the most versatile immunoglobulin because it carries out all of the functions of immunoglobulin molecules in some embodiments. IgG is the major Ig in serum, and the only class of Ig that crosses the placenta. IgG also fixes complement, although the IgG4 subclass does not. Macrophages, monocytes, polymorphonuclear leukocytes (PMNs), and some lymphocytes have receptors for the Fc region of IgG. Not all subclasses bind equally well: IgG2 and IgG4 do not bind to Fc receptors. A consequence of binding to the Fc receptors on PMNs, monocytes, and macrophages is that the cell now internalizes the antigen better in some cases. IgG is an opsonin that enhances phagocytosis. Binding of IgG to Fc receptors on other types of cells results in the activation of other functions.

In certain embodiments, the FcR is a native sequence human FcR. Moreover, a preferred FcR is one that binds an IgG antibody (a gamma (“γ”) receptor) and includes receptors of the FcγRI (CD64), FcγRII (CD32), and FcγRIII (CD16) subclasses, including allelic variants and alternatively spliced forms of these receptors FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain.

“Antibody-dependent cell-mediated cytotoxicity” or “ADCC” refers to a form of cytotoxicity in which secreted Ig bound to Fc receptors (FcRs) present on certain cytotoxic cells (e.g., Natural Killer (NK) cells (also known as large granular lymphocytes (LGL)), neutrophils, and macrophages) enable these cytotoxic effector cells to bind specifically to an antigen-bearing target cell and subsequently kill the target cell with cytotoxins. The antibodies “arm” the cytotoxic cells and are required for such killing. The primary cells for mediating ADCC, NK cells, express FcγRIII only, whereas monocytes express FcγRI, FcγRII, and FcγRIII. To assess ADCC activity of a molecule of interest, an in vitro ADCC assay is performed in some embodiments. Useful effector cells for such assays include peripheral blood mononuclear cells (PBMC) and Natural Killer (NK) cells.

Alternatively, or additionally, in some embodiments, ADCC activity of the molecule of interest is assessed in vivo, e.g., in an animal model.

For certain therapeutic applications, the internalization process is employed for purposes of killing or decreasing the activity or proliferation of a target cell that expresses a LILRB2 protein. The number of antibody molecules internalized will be sufficient or adequate to kill a cell or inhibit its growth. Depending on the potency of an antibody or antibody conjugate, in some instances, the uptake of a single antibody molecule into the cell is sufficient to kill the target cell to which the antibody binds. For example, certain toxins are highly potent in killing such that internalization of one molecule of the toxin conjugated to the antibody is sufficient to kill the targeted cell.

In some embodiments, the LILRB2/1 antibody or antigen-binding fragment provided herein is conjugated or linked to a therapeutic moiety, an imaging or detectable moiety, or an affinity tag. Methods for conjugating or linking polypeptides are well known in the art. Associations (binding) between compounds and labels include any means known in the art including, but not limited to, covalent and non-covalent interactions, chemical conjugation, as well as recombinant techniques. An antibody or antigen-binding fragment thereof is conjugated to, or recombinantly engineered with, an affinity tag (e.g., a purification tag), in some embodiments. Affinity tags such as, for example, poly-histidine (e.g., His6) tags are conventional in the art.

In some embodiments, the LILRB2/1 antibody or antigen-binding fragment further comprises a detectable moiety. Detections accomplished, for example, in vitro, in vivo, or ex vivo. In vitro assays for the detection and/or determination (quantification, qualification, etc.) of, e.g., hLILRB2 protein expressed by macrophages using the antibodies or antigen-binding fragments thereof include but are not limited to, for example, ELISAs, RIAs, and western blots. In some embodiments, in vitro detection, diagnosis, or monitoring of the antigen of the antibodies occurs by obtaining a sample (e.g., a blood sample) from a subject and testing the sample in, for example, a standard ELISA assay.

### Derivatives

Derivatives of the LILRB2/1 antibody products described herein are also provided. The derivatized antibody product can comprise any molecule or substance that imparts a desired property to the antibody product, such as increased half-life in a particular use. The derivatized antibody product can comprise, for example, a detectable (or labeling) moiety (e.g., a radioactive, colorimetric, antigenic or enzymatic molecule, a detectable bead (such as a magnetic or electrodense (e.g., gold) bead), or a molecule that binds to another molecule (e.g., biotin or streptavidin)), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses). Examples of molecules that can be used to derivatize an antibody product include albumin (e.g., human serum albumin) and polyethylene glycol (PEG). Albumin-linked and PEGylated derivatives of antibody products can be prepared using techniques well known in the art. The antibody can be conjugated or otherwise linked to transthyretin (TTR) or a TTR variant. The TTR or TTR variant can be chemically modified with, for example, a chemical selected from the group consisting of dextran, poly(n-vinyl pyrrolidone), polyethylene glycols, polypropylene glycol homopolymers, polypropylene oxide/ethylene oxide co-polymers, polyoxyethylated polyols and polyvinyl alcohols.

Other derivatives include covalent or aggregative conjugates of LILRB2/1 antibody products, with other proteins or polypeptides, such as by expression of recombinant fusion proteins comprising heterologous polypeptides fused to the N-terminus or C-terminus of an LILRB2/1 antibody product. For example, the conjugated peptide can be a heterologous signal (or leader) polypeptide, e.g., the yeast alpha-factor leader, or a peptide such as an epitope tag. LILRB2/1 antibody product-containing fusion proteins can comprise peptides added to facilitate purification or identification of the LILRB2/1 antibody product (e.g., poly-His). An LILRB2/1 antibody product also can be linked to the FLAG peptide as described in Hopp et al., Biol Technology 1988 6:1204-10, and U.S. Pat. No. 5,011,912. The FLAG peptide is highly antigenic and provides an epitope reversibly bound by a specific monoclonal antibody (mAb), enabling rapid assay and facile purification of expressed recombinant protein. Reagents useful for preparing fusion proteins in which the FLAG peptide is fused to a given polypeptide are commercially available (Sigma, St. Louis, Mo.).

Oligomers that contain one or more LILRB2/1 antibody products can be employed as LILRB2 antagonists. Oligomers can be in the form of covalently linked or non-covalently linked dimers, trimers, or higher. Oligomers comprising two or more LILRB2/1 antibody products are contemplated for use, with one example being a homodimer. Other oligomers include heterodimers, homotrimers, heterotrimers, homotetramers, heterotetramers, etc.

Oligomers can comprise multiple LILRB2/1 antibody products joined via covalent or non-covalent interactions between peptide moieties fused to the LILRB2/1 antibody polypeptides. Such peptides can be peptide linkers (spacers), or peptides that have the property of promoting oligomerization. Leucine zippers and certain polypeptides derived from antibodies are among the peptides that can promote oligomerization of LILRB2/1 antibody products attached thereto, as described in more detail below.

Oligomers can comprise from two to four LILRB2/1 antibody products. The LILRB2 product moieties of the oligomer can be in any of the forms described above, e.g., variants or fragments. The oligomers comprise LILRB2/1 antibody products that have LILRB2 binding activity.

Preparation of fusion proteins comprising heterologous polypeptides fused to various portions of antibody-derived polypeptides (including the Fc domain) has been described, e.g., by Ashkenazi et al., Proc Natl Acad Sc/USA. 1991 88(23):10535-9; Byrn et al., Nature. 1990 344(6267):667-70; and Hollenbaugh and Aruffo, Curr Protoc Immunol. 2002 48(1):4:10.19.1-10.19.11.

Dimers are provided comprising two fusion proteins created by fusing a LILRB2 binding fragment of an LILRB2/1 antibody to the Fc region of an antibody. The dimer can be made by, for example, inserting a gene fusion encoding the fusion protein into an appropriate expression vector, expressing the gene fusion in host cells transformed with the recombinant expression vector, and allowing the expressed fusion protein to assemble much like antibody molecules, whereupon interchain disulfide bonds form between the Fc moieties to yield the dimer.

The term “Fc polypeptide” as used herein includes native and mutein forms of polypeptides derived from the Fc region of an antibody. Truncated forms of such polypeptides containing the hinge region that promotes dimerization also are included. Fusion proteins comprising Fc moieties (and oligomers formed therefrom) offer the advantage of facile purification by affinity chromatography over Protein A or Protein G columns.

One exemplary Fc polypeptide, described in PCT Pub. No. WO 93/10151 and U.S. Pat. Nos. 5,426,048 and 5,262,522 (each of which is hereby incorporated by reference), is a single chain polypeptide extending from the N-terminal hinge region to the native C-terminus of the Fc region of a human IgG1 antibody. Another exemplary Fc polypeptide is the Fc mutein described in U.S. Pat. No. 5,457,035 and in Baum et al., EMBO J. 1994 13:3992-4001. The amino acid sequence of this mutein is identical to that of the native Fc sequence presented in PCT Pub. No. WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors.

Alternatively, the oligomer is a fusion protein comprising multiple LILRB2/1 antibody polypeptides, with or without peptide linkers (spacer peptides). Among the suitable peptide linkers are those described in U.S. Pat. Nos. 4,751,180 and 4,935,233.

Another method for preparing oligomeric LILRB2/1 antibody product derivatives involves use of a leucine zipper. Leucine zipper domains are peptides that promote oligomerization of the proteins in which they are found. Examples of leucine zipper domains suitable for producing soluble oligomeric proteins are described in PCT Pub. No. WO 94/10308, and the leucine zipper derived from lung surfactant protein D (SPD) described in Hoppe et al., FEBS Lett. 1994 344:191-5. The use of a modified leucine zipper that allows for stable trimerization of a heterologous protein fused thereto is described in Fanslow et al., Semin Immunol. 1994 6:267-78. Generally, recombinant fusion proteins comprising an LILRB2/1 antibody fragment fused to a leucine zipper peptide are expressed in suitable host cells, and the soluble oligomeric LILRB2/1 antibody products that form are recovered from the culture supernatant.

LILRB2/1 antibody products described herein can also be derivatized or modified such that the products have a longer half-life as compared to the underivatized or unmodified antibody. For example, the antibody product can contain point mutations to increase serum half-life, such as described in PCT Pub. No. WO 00/09560.

### Nucleic Acids and Cells

Nucleic acids are provided that encode one or more chains of an antibody product herein, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing are also provided.

Nucleic acids provided encode antibody products disclosed herein, such as a light chain variable region shown in Table 1 or in Table 3, and/or a heavy chain variable region shown in Table 1 or in Table 3. Due to the degeneracy of the genetic code, each of the polypeptide sequences listed in Table 1 or in Table 3 is also encoded other nucleic acid sequences besides those listed in Table 1 or in Table 3. The present disclosure provides each degenerate nucleotide sequence encoding each antibody product.

The term “polynucleotide” or “nucleic acid” means single-stranded or double-stranded polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2″,3′-dideoxyribose, and internucleotide linkage modifications such as phosphorothioate, phosphorodithioate, phosphoroselenoate, phosphorodiselenoate, phosphoroanilothioate, phoshoraniladate, and phosphoroamidate. The term includes both single and double stranded forms.

An “isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences can include, in addition to the specified sequences, coding sequences for up to ten or even up to twenty other proteins or portions thereof, or can include operably linked regulatory sequences that control expression of the coding region of the recited nucleic acid sequences, and/or can include vector sequences.

Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences”.

The term “control sequence” refers to a polynucleotide sequence that can affect the expression and processing of coding sequences to which it is ligated. The nature of such control sequences can depend upon the host organism. For example, control sequences for eukaryotes can include promoters comprising one or a plurality of recognition sites for transcription factors, transcription enhancer sequences, and transcription termination sequence. “Control sequences” can include leader sequences and/or fusion partner sequences.

The term “vector” means any molecule or entity (e.g., nucleic acid, plasmid, bacteriophage, or virus) used to transfer protein coding information into a host cell.

The term “expression vector” or “expression construct” refers to a vector that is suitable for transformation of a host cell and contains nucleic acid sequences that direct and/or control (in conjunction with the host cell) expression of one or more heterologous coding regions operatively linked thereto. An expression construct can include, but is not limited to, sequences that affect or control transcription, translation, and, if introns are present, affect RNA splicing of a coding region operably linked thereto.

As used herein, “operably linked” means that the components to which the term is applied are in a relationship that allows them to carry out their inherent functions under suitable conditions. For example, a control sequence in a vector that is “operably linked” to a protein coding sequence is ligated thereto so that expression of the protein coding sequence is achieved under conditions compatible with the transcriptional activity of the control sequences.

The term “host cell” means a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

DNA encoding antibody polypeptides (e.g., heavy or light chain, variable domain only, or full length) can be isolated from B-cells of mice that have been immunized with LILRB2 or an immunogenic fragment thereof. The DNA can be isolated by conventional procedures such as polymerase chain reaction (PCR). Phage display is another example of a known technique whereby nucleotide sequences encoding antibody polypeptides can be selected.

Nucleic acids are provided that hybridize to other nucleic acids under particular hybridization conditions. Methods for hybridizing nucleic acids are well-known in the art. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C., in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequences that are at least 65, 70, 75, 80, 85, 90, 95, 96, 97, 98 or 99% identical to each other typically remain hybridized to each other.

The basic parameters affecting the choice of hybridization conditions and guidance for devising suitable conditions are set forth by, for example, Sambrook, Fritsch, and Maniatis (1989, Molecular Cloning: A Laboratory Manual, Cold Spring Harbor Laboratory Press, Cold Spring Harbor, N.Y., Chapters 9 and 11; and Current Protocols in Molecular Biology, 1995, Ausubel et al., eds., John Wiley & Sons, Inc., sections 2.10 and 6.3-6.4), and can be readily determined by those having ordinary skill in the art based on, for example, the length and/or base composition of the DNA.

Changes can be introduced by mutation into a nucleic acid, thereby leading to changes in the amino acid sequence of a polypeptide (e.g., an antibody or antibody derivative) that it encodes. Mutations can be introduced using any technique known in the art. One or more particular amino acid residues can be changed using, for example, a site-directed mutagenesis protocol. One or more randomly selected residue can be changed using, for example, a random mutagenesis protocol. However it is made, a mutant polypeptide can be expressed and screened for a desired property.

Polypeptides that are components of an antibody product of interest are expressed in any suitable recombinant expression system.

Expression vectors are provided comprising nucleic acid encoding a LILRB2/1 antibody product. Examples of vectors include, but are not limited to, plasmids, viral vectors, non-episomal mammalian vectors and expression vectors, for example, recombinant expression vectors.

Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element.

Optionally, the vector can contain a “tag”-encoding sequence, that is, an oligonucleotide molecule located at the 5′ or 3′ end of the coding sequence, the oligonucleotide sequence encoding polyHis (such as hexaHis), or another “tag” for which commercially available antibodies exist, such as FLAG, HA (hemagglutinin from influenza virus), or myc. The tag is typically fused to the antibody protein upon expression, and can serve as a means for affinity purification of the antibody from the host cell. Affinity purification can be accomplished, for example, by column chromatography using antibodies against the tag as an affinity matrix. Optionally, the tag can subsequently be removed from the purified antibody polypeptide by various means such as using certain peptidases for cleavage.

Flanking sequences in the expression vector can be homologous (i.e., from the same species and/or strain as the host cell), heterologous (i.e., from a species other than the host cell species or strain), hybrid (i.e., a combination of flanking sequences from more than one source), synthetic or native. As such, the source of a flanking sequence can be any prokaryotic or eukaryotic organism, any vertebrate or invertebrate organism, or any plant, provided that the flanking sequence is functional in, and can be activated by, the host cell machinery.

Expression and cloning vectors typically contain a promoter that is recognized by the host organism and operably linked to nucleic acid encoding the LILRB2/1 antibody product. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, initiate continuous gene product production; that is, there is little or no experimental control over gene expression. A large number of promoters, recognized by a variety of potential host cells, are well known. A suitable promoter is operably linked to the DNA encoding the LILRB2/1 antibody product by removing the promoter from the source DNA by restriction enzyme digestion or amplifying the promoter by polymerase chain reaction and inserting the desired promoter sequence into the vector.

Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus and—most preferably Simian Virus 40 (SV40). Other suitable mammalian promoters include heterologous mammalian promoters, for example, heat-shock promoters and the actin promoter.

Exemplary promoters useful in recombinant expression vectors include, but are not limited to: the SV40 early promoter region (Bemoist and Chambon, 1981, Nature, 290:304-10); the CMV promoter; the promoter contained in the 3′ long terminal repeat of Rous sarcoma virus (Yamamoto, et al., Cell. 1980 22:787-97); the herpes thymidine kinase promoter (Wagner et al., Proc Natl Acad Sci USA. 1981 78:1444-5); the regulatory sequences of the metallothionine gene (Brinster et al., Nature. 1982 296:39-42); prokaryotic expression vectors such as the beta-lactamase promoter (Villa-Komaroff et al., Proc Natl Acad Sci USA1978 75:3727-31); or the tac promoter (DeBoer et al., Proc Natl Acad Sci USA. 1983 80:21-5). Also available for use are the following animal transcriptional control regions, which exhibit tissue specificity and have been utilized in transgenic animals: the elastase I gene control region that is active in pancreatic acinar cells (Swift et al., Cell. 1984 38:639-46; Ornitz et al., Cold Spring Harb Symp Quant Biol. 1986 50:399-409; MacDonald, Hepatology. 1987 7:425-515); the insulin gene control region that is active in pancreatic beta cells (Hanahan, Nature. 1985 315:115-22); the mouse mammary tumor virus control region that is active in testicular, breast, lymphoid and mast cells (Leder et al., Cell. 1986 45:485-95); the albumin gene control region that is active in liver (Pinkert et al., Genes Dev 1987 1:268-76); the alpha-feto-protein gene control region that is active in liver (Krumlauf et al., Mol Cell Biol. 1985 5:1639-48; Hammer et al., Science. 1987 235:53-8); the alpha 1-antitrypsin gene control region that is active in the liver (Kelsey et al., Genes Dev. 1987 1:161-71); the beta-globin gene control region that is active in myeloid cells (Mogram et al., Nature. 1985 315:338-40; Kollias et al., Cell. 1986 46:89-94); the myelin basic protein gene control region that is active in oligodendrocyte cells in the brain (Readhead et al., Cell. 1987 48:703-12); the myosin light chain-2 gene control region that is active in skeletal muscle (Sani, Nature. 1985 314:283-6); the gonadotropic releasing hormone gene control region that is active in the hypothalamus (Mason et al., Science. 1986 234:1372-8); and most particularly the immunoglobulin gene control region that is active in lymphoid cells (Grosschedl et al., Cell. 1984 38:647-58; Adams et al., Nature. 1985 318:533-8; Alexander et al., Mol Cell Biol. 1987 7:1436-44).

An enhancer sequence can be inserted into the vector to increase the transcription in higher eukaryotes of a nucleic acid encoding an LILRB2/1 antibody product described herein. Various enhancer sequences available from mammalian genes are known (e.g., globin, elastase, albumin, alpha-feto-protein and insulin). An enhancer sequence from a virus also can be used. The SV40 enhancer, the cytomegalovirus early promoter enhancer, the polyoma enhancer, and adenovirus enhancers are exemplary enhancing elements for the activation of eukaryotic promoters. While an enhancer can be spliced into the vector at a position 5′ or 3′ to a nucleic acid molecule, it is typically placed at a site 5′ to the promoter.

In expression vectors, a transcription termination sequence is typically located 3′ of the end of a polypeptide-coding region and serves to terminate transcription. A transcription termination sequence used for expression in prokaryotic cells typically is a G-C rich fragment followed by a poly-T sequence. While the sequence is easily cloned from a library or even purchased commercially as part of a vector, it can also be readily synthesized using methods for nucleic acid synthesis such as those described herein.

Selection genes can be used to amplify the gene that will be expressed. Amplification is a process whereby genes that cannot in single copy be expressed at high enough levels to permit survival and growth of cells under certain selection conditions are reiterated in tandem within the chromosomes of successive generations of recombinant cells. Examples of suitable amplifiable selectable markers for mammalian cells include dihydrofolate reductase (DHFR) and promoterless thymidine kinase. In the use of these markers mammalian cell transformants are placed under selection pressure in which only the transformants are uniquely adapted to survive by virtue of the selection gene present in the vector. Selection pressure is imposed by culturing the transformed cells under conditions in which the concentration of selection agent in the medium is successively increased, thereby permitting survival of only those cells in which the selection gene has been amplified. Under these circumstances, DNA adjacent to the selection gene, such as DNA encoding an antibody, is co-amplified with the selection gene. As a result, increased quantities of LILRB2 polypeptide are synthesized from the amplified DNA.

A ribosome-binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the polypeptide to be expressed.

In some cases, for example where glycosylation is desired in a eukaryotic host cell expression system, various presequences can be manipulated to improve glycosylation or yield. For example, the peptidase cleavage site of a particular signal peptide can be altered, or pro-sequences added, which also can affect glycosylation. The final protein product can have, in the −1 position (relative to the first amino acid of the mature protein) one or more additional amino acids incident to expression, which may not have been totally removed. For example, the final protein product can have one or two amino acid residues found in the peptidase cleavage site, attached to the amino terminus. Alternatively, use of some enzyme cleavage sites can result in a slightly truncated yet active form of the desired polypeptide, if the enzyme cuts at such area within the mature polypeptide.

Where a commercially available expression vector lacks some of the desired flanking sequences as described above, the vector can be modified by individually ligating these sequences into the vector. After the vector has been chosen and modified as desired, a nucleic acid molecule encoding an LILRB2/1 antibody product is inserted into the proper site of the vector.

The completed vector containing sequences encoding the antibody product is inserted into a suitable host cell for amplification and/or polypeptide expression. The transformation of an expression vector for an LILRB2/1 antibody product into a selected host cell can be accomplished by well-known methods including methods such as transfection, infection, calcium chloride, electroporation, microinjection, lipofection, DEAE-dextran method, or other known techniques. The method selected will in part be a function of the type of host cell to be used. These methods and other suitable methods are well known to the skilled artisan.

Antibodies can be expressed in hybridoma cell lines or in cell lines other than hybridomas. Expression constructs encoding the antibodies can be used to transform a mammalian, insect or microbial host cell. Transformation can be performed using any known method for introducing polynucleotides into a host cell, including, for example packaging the polynucleotide in a virus or bacteriophage and transducing a host cell with the construct by transfection procedures known in the art, as exemplified by U.S. Pat. Nos. 4,399,216, 4,912,040, 4,740,461, and 4,959,455. The optimal transformation procedure used will depend upon which type of host cell is being transformed. Methods for introduction of heterologous polynucleotides into mammalian cells are well known in the art and include, but are not limited to, dextran-mediated transfection, calcium phosphate precipitation, polybrene mediated transfection, protoplast fusion, electroporation, encapsulation of the polynucleotide(s) in liposomes, mixing nucleic acid with positively charged lipids, and direct microinjection of the DNA into nuclei.

The transformed host cell, when cultured under appropriate conditions, synthesizes an LILRB2/1 antibody product that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Mammalian cell lines available as hosts for expression are well known in the art and include, but are not limited to, many immortalized cell lines available from the American Type Culture Collection (ATCC), such as Chinese hamster ovary (CHO) cells, HeLa cells, baby hamster kidney (BHK) cells, monkey kidney cells (COS), human hepatocellular carcinoma cells (e.g., Hep G2), human embryonic kidney cells (HEK) (e.g., HEK-293) and a number of other cell lines. The best cell line for expressing a particular DNA construct can be selected by testing various cell lines to determine which ones have the highest levels of expression levels and produce antibody products with the desired LILRB2 binding properties.

Nucleic acid molecules that are suitable for use as primers or hybridization probes for the detection of nucleic acid sequences are also provided. A nucleic acid molecule can comprise only a portion of a nucleic acid sequence encoding a full-length polypeptide, for example, a fragment that can be used as a probe or primer or a fragment encoding an active portion (e.g., a LILRB2 binding portion) of a polypeptide.

### Compositions

Compositions that include LILRB2/1 antibody products are also provided. Pharmaceutical compositions typically include one or more of a buffer, a pharmaceutically acceptable diluent, a carrier, a solubilizer, an emulsifier and a preservative. The use of the foregoing antibody products in the preparation of a pharmaceutical composition or medicament is also provided.

Acceptable formulation components for pharmaceutical preparations are nontoxic to recipients at the dosages and concentrations employed. In addition to the antibody products that are provided herein, compositions can contain components for modifying, maintaining or preserving, for example, the pH, osmolarity, viscosity, clarity, color, isotonicity, odor, sterility, stability, rate of dissolution or release, adsorption or penetration of the composition. Suitable materials for formulating pharmaceutical compositions include, but are not limited to, amino acids (such as glycine, glutamine, asparagine, arginine or lysine); antimicrobials; antioxidants (such as ascorbic acid, sodium sulfite or sodium hydrogen-sulfite); buffers (such as acetate, borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (see Remington's Pharmaceutical Sciences, 23rd ed., (Adejare, ed.), 2020, Elsevier Academic Press).

The primary vehicle or carrier in a pharmaceutical composition can be either aqueous or non-aqueous in nature. Suitable vehicles or carriers for such compositions include water for injection, physiological saline solution or artificial cerebrospinal fluid, possibly supplemented with other materials common in compositions for parenteral administration. Neutral buffered saline or saline mixed with serum albumin are further exemplary vehicles. Compositions comprising LILRB2/1 antibody product can be prepared for storage by mixing the selected composition having the desired degree of purity with optional formulation agents in the form of a lyophilized cake or an aqueous solution. Further, the LILRB2/1 antibody product can be formulated as a lyophilizate using appropriate excipients such as sucrose.

Formulation components are present in concentrations that are acceptable to the site of administration. Buffers are advantageously used to maintain the composition at physiological pH or at a slightly lower pH, typically within a pH range of from about 4.0 to about 8.5, or alternatively, between about 5.0 to 8.0. Pharmaceutical compositions can comprise TRIS buffer of about pH 6.5-8.5, or acetate buffer of about pH 4.0-5.5, which can further include sorbitol or a suitable substitute therefor.

Additional pharmaceutical compositions are in the form of sustained- or controlled-delivery formulations. Techniques for formulating a variety of other sustained- or controlled-delivery means, such as liposome carriers, bio-erodible microparticles or porous beads and depot injections can be used (see, e.g., PCT Pub. No. WO 93/15722 A1, which describes the controlled release of porous polymeric microparticles for the delivery of pharmaceutical compositions). Sustained-release preparations can include semipermeable polymer matrices in the form of shaped articles, e.g., films, or microcapsules, polyesters, hydrogels, polylactides (U.S. Pat. No. 3,773,919 and EP 058,481), copolymers of L-glutamic acid and gamma ethyl-L-glutamate (Sidman et al., Biopolymers. 1983 22:547-56), poly (2-hydroxyethyl-methacrylate) (Langer et al., J Biomed Mater Res. 1981 15:167-277) and Langer, Chem Tech. 1982 12:98-105), ethylene vinyl acetate (Langer et al., ibid.) or poly-D(−)-3-hydroxybutyric acid (EP 133,988). Sustained release compositions can also include liposomes, which can be prepared by any of several methods known in the art. See, e.g., Eppstein et al., Proc Natl Acad Sci USA. 1985 82:3688-92; EPO Pub. Nos. EP 036676; EP 088046, and EP 143949.

Once the pharmaceutical composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) that is reconstituted prior to administration.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Kits are provided for multi-dose or single-dose administration units. For example, kits can each contain both a first container having a dried protein and a second container having an aqueous diluent, including for example single and multi-chambered pre-filled syringes (e.g., liquid syringes, lyosyringes or needle-free syringes).

The pharmaceutical compositions can be delivered parenterally, typically by injection. Injections can be intraocular, intraperitoneal, intraportal, intramuscular, intravenous, intrathecal, intracerebral (intra-parenchymal), intracerebroventricular, intraarterial, intralesional, perilesional, or subcutaneous. Eye drops can be used for intraocular administration. In some instances, injections can be localized to the vicinity of a particular bone or bones to which the treatment is targeted. For parenteral administration, the antibodies can be administered in a pyrogen-free, parenterally acceptable aqueous solution comprising the desired LILRB2/1 antibody product in a pharmaceutically acceptable vehicle. A particularly suitable vehicle for parenteral injection is sterile distilled water in which the LILRB2/1 antibody product are formulated as a sterile, isotonic solution, properly preserved.

Pharmaceutical compositions comprising the subject LILRB2/1 antibody products can be administered by bolus injection or continuously by infusion, by implantation device, sustained release systems or other means for accomplishing prolonged release. The pharmaceutical composition also can be administered locally via implantation of a membrane, sponge or another appropriate material onto which the desired molecule has been absorbed or encapsulated. Where an implantation device is used, the device can be implanted into any suitable tissue or organ, and delivery of the desired molecule can be via diffusion, timed-release bolus, or continuous release. The preparation can be formulated with agents, such as injectable microspheres, bio-erodible particles, polymeric compounds (such as polylactic acid; polyglycolic acid; or copoly (lactic/glycolic) acid (PLGA), beads or liposomes, that can provide controlled or sustained release of the product which can then be delivered via a depot injection. Formulation with hyaluronic acid has the effect of promoting sustained duration in the circulation.

Subject compositions comprising a LILRB2/1 antibody product also can be used ex vivo. In such instances, cells, tissues, or organs that have been removed from the patient are exposed to or cultured with the LILRB2/1 antibody product. The cultured cells can then be implanted back into the patient or a different patient or used for other purposes.

A LILRB2/1 antibody product can be delivered by implanting certain cells that have been genetically engineered, using methods such as those described herein, to express and secrete the polypeptide. Such cells can be animal or human cells, and can be autologous, heterologous, or xenogeneic, or can be immortalized. In order to decrease the chance of an immunological response, the cells can be encapsulated to avoid infiltration of surrounding tissues. Encapsulation materials are typically biocompatible, semi-permeable polymeric enclosures or membranes that allow the release of the protein product(s) but prevent the destruction of the cells by the patient's immune system or by other detrimental factors from the surrounding tissues.

As used herein, “substantially pure” means that the described species of molecule is the predominant species present, that is, on a molar basis it is more abundant than any other individual species in the same mixture. A substantially pure molecule can be a composition in which the object species comprises at least 50% (on a molar basis) of all macromolecular species present. A substantially pure composition can comprise at least 80%, 85%, 90%, 95%, or 99% of all macromolecular species present in the composition. The object species can also be purified to essential homogeneity in which contaminating species cannot be detected in the composition by conventional detection methods and thus the composition consists of a single detectable macromolecular species.

### Dosages

The pharmaceutical compositions that are provided can be administered for prophylactic and/or therapeutic treatment.

As used herein, the terms “treatment,” “treating,” and the like, refer to administering an agent or carrying out a procedure, for the purposes of obtaining an effect. The effect is prophylactic in terms of completely or partially preventing a disease or symptom thereof and/or is therapeutic in terms of effecting a partial or complete cure for a disease and/or symptoms of the disease. “Treatment,” as used herein, includes treatment of a disease or disorder (e.g., cancer) in a mammal, particularly in a human, and includes: (a) preventing the disease or a symptom of a disease from occurring in a subject which is predisposed to the disease but has not yet been diagnosed as having it (e.g., including diseases that are associated with or caused by a primary disease); (b) inhibiting the disease, i.e., arresting its development; and (c) relieving the disease, i.e., causing regression of the disease. Treating refers to any clinical indicia of success in the treatment or amelioration or prevention, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters, including the results of an examination by a physician. Accordingly, the term “treating” includes the administration of the compounds or agents of the present disclosure to prevent or delay, to alleviate, or to arrest or inhibit development of the symptoms or conditions associated with diseases (e.g., cancer). The term “therapeutic effect” refers to the reduction, elimination, or prevention of the disease, symptoms of the disease, or side effects of the disease in the subject. For example, a subject is “treated” for a disease or disorder if, after receiving a therapeutic amount of a combination of a LILRB2/1 antibody product provided herein, the patient shows one or more observable and/or measurable changes in an endpoint or symptom of the disease condition.

An “effective response” in accordance with the present disclosure is achieved when the subject experiences partial or total alleviation or reduction of signs or symptoms of illness and, in the case of the treatment of cancer, specifically includes, without limitation, amelioration of symptoms, retarding progression, cure, remission, prolongation of survival, or other objective responses. The expected progression-free survival times can be measured in months to years, depending on prognostic factors including the number of relapses, stage of disease, and other factors. Prolonging survival includes without limitation times of at least 1 month (mo.), about at least 2 mos., about at least 3 mos., about at least 4 mos., about at least 6 mos., about at least 1 year, about at least 2 years, about at least 3 years, etc. Overall survival is also measured, for example, in months to years. Alternatively, an effective response can be that a subject's symptoms remain static.

Administration of a therapeutic agent in a prophylactic method occurs prior to the manifestation of symptoms of an undesired disease or disorder, such that the disease or disorder is prevented or, alternatively, delayed in its progression. Thus, when used in conjunction with prophylactic methods, the term “therapeutically effective” means that, after treatment, a smaller number of subjects (on average) develop the undesired disease or disorder or progress in severity of symptoms.

The terms “recipient,” “individual,” “subject,” “host,” and “patient,” are used interchangeably herein and refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys, etc. The mammal can be a human.

In general, toxicity and therapeutic efficacy of the antibody product can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with little or no toxicity. The dosage can vary within this range depending upon the dosage form employed and the route of administration utilized.

The effective amount of a pharmaceutical composition comprising a LILRB2/1 antibody product to be employed therapeutically or prophylactically will depend, for example, upon the therapeutic context and objectives. One skilled in the art will appreciate that the appropriate dosage levels for treatment, will thus vary depending, in part, upon the molecule delivered, the indication for which the LILRB2/1 antibody is being used, the route of administration, and the size (body weight, body surface or organ size) and/or condition (the age and general health) of the patient. A clinician can titer the dosage and modify the route of administration to obtain the optimal therapeutic effect. Typical dosages range from about 1 mg/kg to up to about 1600 mg/kg or more, depending on the factors mentioned above. The dosage can range from 1 mg/kg up to about 200 mg/kg; or 1 mg/kg up to about 1200 mg/kg; or 1 μg/kg up to about 1600 mg/kg.

The dosing frequency will depend upon the pharmacokinetic parameters of the LILRB2/1 antibody product in the formulation. For example, a clinician will administer the composition until a dosage is reached that achieves the desired effect. The composition can therefore be administered as a single dose, or as two or more doses (which can contain the same amount of the desired molecule) over time, or as a continuous infusion via an implantation device or catheter. Treatment can be continuous over time or intermittent. Further refinement of the appropriate dosage is routinely made by those of ordinary skill in the art and is within the ambit of tasks routinely performed by them. Appropriate dosages can be ascertained through use of appropriate dose-response data. An exemplary dosing schedule is every 2 to 3 weeks.

To treat a disease condition by targeting LILRB2, a composition comprising the LILRB2/1 antibody product is administered to the patient in an amount and for a time sufficient to induce a sustained improvement in at least one indicator that reflects the severity of the disorder. An improvement is considered “sustained” if the patient exhibits the improvement on at least two occasions separated by at least one to seven days, or in some instances one to six weeks. The appropriate interval will depend to some extent on what disease condition is being treated; it is within the purview of the skilled physician to determine the appropriate interval for determining whether the improvement is sustained. The degree of improvement is determined based on signs or symptoms, and can also employ questionnaires that are administered to the patient, such as quality-of-life questionnaires.

Various indicators that reflect the extent of the patient's illness can be assessed for determining whether the amount and time of the treatment is sufficient. The baseline value for the chosen indicator or indicators is established by examination of the patient prior to administration of the first dose of antibody. Preferably, the baseline examination is done within about 60 days of administering the first dose. If the antibody is being administered to treat acute symptoms, such as for example to treat a broken bone, the first dose is administered as soon as practically possible after the injury has occurred.

Improvement is induced by administering the LILRB2/1 antibody product until the patient manifests an improvement over baseline for the chosen indicator or indicators. In treating chronic conditions, this degree of improvement is obtained by repeatedly administering this medicament over a period of at least a month or more, e.g., for one, two, or three months or longer, or indefinitely. A period of one to six weeks, or even a single dose, often is sufficient for treating acute conditions. For injuries or acute conditions, a single dose can be sufficient.

Although the extent of the patient's illness after treatment may appear improved according to one or more indicators, treatment can be continued indefinitely at the same level or at a reduced dose or frequency. Once treatment has been reduced or discontinued, it later can be resumed at the original level if symptoms should reappear.

### Methods of Use

The LILRB2/1 antibody products disclosed herein have a variety of utilities. Some of the antibodies and fragments, for instance, are useful for specific binding assays, affinity purification of LILRB2 or its ligands, and screening assays to identify other antagonists of LILRB2 activity. The antibody products can be used to treat various diseases that are associated with the activity of LILRB2.

LILRB2/1 antibody products can be used to detect LILRB2 in biological samples. Such uses allow the identification of cells or tissues that produce the protein or serve as a diagnostic for detecting pathological conditions in which LILRB2 is overproduced or underproduced. The strong expression of LILRB2 by macrophages, osteoclasts, and other myeloid cells can be a marker of the activity of those cells, and detection of LILRB2 expression on myeloid cells can be used as a marker of a disease or disorder characterized by the cell type in question. Likewise, detection of LILRB2 expression by cancer cells can be used to identify subjects whose cancer may be amenable to treatment with therapeutic LILRB2/1 antibody product methods disclosed herein.

Accordingly, methods are provided of detecting cell activity in a biological sample, such as a sample of an in vitro medium or a tissue sample from a subject, or in vivo in a subject, in which the method comprises contacting the cell expressing LILRB1 and/or LILRB2 with an LILRB2/1 antibody product provided herein. The LILRB2/1 antibody product can be conjugated to a detectable moiety, and the method comprises detecting the moiety directly. The method can comprise detecting binding of the LILRB2/1 antibody product to the cell indirectly by way of a detectably moiety that binds to the antibody. For example, an IgG antibody conjugated to a detectable moiety can be used to bind to the LILRB2/1 antibody presented as an IgG isotype. The cell can be a tumor cell. The cell can be a myeloid cell (e.g., a monocyte, dendritic cell, macrophage, myeloid-derived suppressor cell, tumor-associated macrophage, immunosuppressive macrophage or M2-like macrophage) or an osteoclast.

The antibody products provided can also be used in methods to screen for a molecule that binds to LILRB2. A variety of competitive screening methods, for example, can be used. In some methods, a LILRB2 molecule or fragment thereof to which an LILRB2/1 antibody product binds, is contacted with an antibody product disclosed herein together with another molecule (i.e., a candidate molecule). A reduction in binding between the antibody product and LILRB2 is an indication that the candidate molecule binds LILRB2. Binding of the antibody product can be detected using a variety of methods, e.g., an ELISA. Detection of binding between the LILRB2/1 antibody product and LILRB2 can be simplified by detectably labeling the antibody. In some methods, a molecule that exhibits binding in the initial screen is further analyzed to determine whether it inhibits or modulates a LILRB2 activity.

The LILRB2/1 antibody products provided herein are useful for the treatment of human disease, including cancers.

LILRB2/1 antibody products described herein can be used for the treatment of cancer, either alone or in combination with another anti-cancer therapeutic. Cancers to be treated are those where the cancer cells are known to express LILRB2 or are of a type that has previously been observed to express LILRB2. Certain cancers that are EGFR-mutant have been found to more highly express LILRB2, and so are contemplated for treatment using the antibodies disclosed herein. In contrast, expression of LILRB2 in cancer cells has also been found to be inversely related to expression of PD-L1. Anti-LILRB2 treatment as described herein is thus also contemplated in cases where PD-L1 expression by the cancer cells is not observed. Such treatment is indicated when therapeutic intervention in the PD-1/PD-L1 axis is, or is expected to be, ineffective.

Provided herein are methods of treating a patient having a cancer, in which a LILRB2/1 antibody product mediates killing of the cells of the cancer.

As used herein, the term “cancer” has its general meaning in the art and includes, but is not limited to, solid tumors and blood-borne tumors. The term cancer includes diseases of the skin, tissues, organs, bone, cartilage, blood and vessels. The term “cancer” further encompasses both primary and metastatic cancers.

Examples of cancers that can be treated by methods and compositions provided herein include, but are not limited to, cancers of the bladder, blood, bone, bone marrow, brain, breast, cervix, colon, esophagus, gastrointestinal tract, rectum, head and neck, kidney, larynx, liver, lung, mouth nasopharynx, neck, ovary, pancreas, prostate, skin, stomach, testis, thyroid, tongue, and uterus.

Cancers to be treated include, for example, acute lymphoblastic leukemia, chronic lymphocytic leukemia, acute myeloid leukemia, myelodysplastic syndrome, chronic myelogenous leukemia, Hodgkin's disease; Hodgkin's lymphoma, non-Hodgkin's lymphoma, B cell lymphoma, Burkitt lymphoma, bladder cancer, breast cancer, cervical cancer, colorectal cancer, endometrial cancer, esophageal cancer, gallbladder cancer, hepatocellular cancer, head and neck cancer, kidney cancer, melanoma, malignant mesothelioma, nasopharyngeal cancer, neuroblastoma, glioblastoma, pancreatic cancer, multiple myeloma, prostate cancer, small cell lung cancer, non-small cell lung cancer, and metastatic cancers. Cancers to be treated include, for example, glioblastoma multiforme, head and neck cancer, kidney renal clear cell cancer, acute myeloid leukemia, pancreatic adenocarcinoma, skin cutaneous melanoma, stomach adenocarcinoma, testicular germ cell cancer, gastric cancer, Merkel cell carcinoma, dendritic sarcoma, non-small cell lung cancer, papillary thyroid cancer, cutaneous squamous cell carcinoma, or ovarian cancer.

In addition, the cancer can specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial adenomatous polyposis; solid carcinoma; carcinoid tumor, malignant; bronchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non-encapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; malignant cellular blue nevus; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides or cutaneous T-cell lymphoma; B cell lymphoma or other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

As an example, a method is provided for the treatment of a subject suffering from melanoma. As used herein, “melanoma” refers to a condition characterized by the growth of a tumor arising from the melanocytic system of the skin and other organs. Most melanocytes occur in the skin, but are also found in the meninges, digestive tract, lymph nodes and eyes. When melanoma occurs in the skin, it is referred to as cutaneous melanoma. Melanoma can also occur in the eyes and is called ocular or intraocular melanoma. Melanoma occurs rarely in the meninges, the digestive tract, lymph nodes or other areas where melanocytes are found.

Cells of a cancer treated in methods provided herein can express LILRB1 and LILRB2. Cells of the cancer can overexpress LILRB1 and LILRB2.

It is thought that some cytomegalovirus (CMV) infection may be a factor in tumorigenesis. CMV can cause cells to express UL18, a cytomegalovirus MHC class I homolog that binds LILRB1 (Yang and Bjorkman, Proc Natl Acad Sci USA. 2008 105(29):10095-100). The antibodies can be used in methods of treatment of cancers in patients who have CMV infection on whom HCMV DNA is detectable in tumor tissues, such as Hodgkin's and non-Hodgkin's lymphoma, colorectal cancer, cervical cancer, and breast cancer. CMV infection causing HLA-G mimic may contribute to immunosuppression in the TME via binding to LILRB1 and LILRB2. LILRB2/1 antibody products can block these interactions and limit or reduce, or reverse or prevent, immunosuppression.

A way by which a LILRB2/1 antibody product can mediate cancer cell killing is through antibody-dependent cellular toxicity (“ADCC”). ADCC is the process by which antibodies coat a target cell (e.g., a cancer cell or bacterial cell) and recruit effector cells to induce target cell death via non-phagocytic mechanisms.

As noted above, in treatment methods the LILRB2/1 antibody products provided herein can be used as a monotherapy or in a combination therapy. A “combination” therapy refers to administration of one treatment agent before, during, or after administration of the other treatment agent to the subject.

In a combination treatment for cancer, the LILRB2/1 antibody product is used in combination with one or more other anticancer modalities for the treatment of a cancer in a subject. As is well understood in the field, such anticancer modalities can comprise surgery, radiation therapy, chemotherapy, and immunotherapy. In some embodiments, the anticancer modality can be a chemotherapeutic or biologic molecule. In some embodiments, the anticancer modality can be an immunotherapeutic molecule. In some embodiments, the immunotherapeutic can be a checkpoint inhibitor. In some embodiments, the checkpoint inhibitor can be a PD-1 antagonist. In some embodiments, the checkpoint inhibitor can be a PD-L1 antagonist.

Provided herein are methods of treating a patient having a cancer, comprising administering to the patient an LILRB2/1 antibody product as described herein (e.g., as a pharmaceutical composition thereof) in combination with at least one other anticancer product (e.g., as a pharmaceutical composition comprising a chemotherapy or an immunotherapy). In some embodiments, such combination use can comprise administering an effective amount of the LILRB2/1 antibody product and an effective amount of the at least one other anticancer product. In some embodiments, such combination use can comprise administering an effective amount of a combination of the LILRB2/1 antibody product and the at least one other anticancer product. In this case, it is possible that the LILRB2/1 antibody product and the at least one other anticancer product complement each other therapeutically and that one or both of them may be administered in subtherapeutic amounts, yet the combined use may yet be therapeutically effective. Such combination use may provide opportunities for clinical use in situations where a therapeutic window for one or another component of the combination is narrow, so to limit or mitigate adverse effects in the patient.

As an example of a combination with an immunotherapy, methods of treating a patient having a cancer are provided, comprising administering to the patient a therapeutically effective amount of a LILRB2/1 antibody product and one or more immune checkpoint inhibitors.

The term “immune checkpoint inhibitor” or “checkpoint inhibitor” generally refers to an agent that modulates an immune checkpoint protein (a “checkpoint protein”). A checkpoint inhibitor can achieve total or partial reduction, inhibition, interference to an activity of the checkpoint protein, or produce other changes to structure of the checkpoint protein that change binding of a checkpoint protein to a ligand, and/or impact a pathway related to activity of the checkpoint protein such as, for example, by acting as an antagonist to a checkpoint protein or a ligand of the checkpoint protein. The immune checkpoint inhibitor can be a compound such as an antibody or other protein that binds and antagonizes human programmed cell death protein 1 (PD-1; aka PDCD1, CD279) or programmed cell death ligand 1 (PD-L1; aka BZ-H1, CD274). Such immune checkpoint inhibitors are termed, respectively, PD-1 antagonists and PD-L1 antagonists.

“PD-1 antagonist” means any chemical compound or biological molecule that blocks binding of PD-1 expressed on an immune cell (T cell, B cell or NKT cell) to PD-L1 expressed on a cancer cell, and preferably also blocks binding of the immune-cell expressed PD-1 to PD-L2 expressed on a cancer cell. Alternative names or synonyms are given for PD-1 and its ligands. For PD-1: PDCD1, PD1, CD279 and SLEB2; for PD-L1: PDCDIL1, PDL1, B7H1, B7-4, CD274 and B7-H; and for PD-L2: PDCDIL2, PDL2, B7-DC, Btdc, and CD273. In any of the treatment methods, medicaments and disclosed uses in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP 005009. Human PD-L1 and PD-L2 amino acid sequences can be found in NCBI Locus No.: NP 054862 and NP 079515, respectively.

As disclosed herein, checkpoint proteins can, in certain contexts and states, interfere with T-cell mediated killing of cancer cells. Checkpoint inhibitors can reverse the interference of checkpoint proteins but interference with a checkpoint protein is not enough in certain types of cancers (e.g., certain solid tumors). The present disclosure contemplates that combining an antibody product provided herein with another a checkpoint inhibitor relieves macrophage-mediated T-cell exhaustion and stimulates T-cell effector function.

A checkpoint inhibitor can inhibit one or more checkpoint proteins. Non-limiting examples of checkpoint proteins include: PD-1, CD28, CTLA-4, ICOS, TMIGD2, 4-1BB, BTLA, CD160, LIGHT, LAG3, OX40, CD27, CD40L, CD47, GITR, DNAM-1, TIGIT, CD96, PVRIG 2B4, TIM-3, Galectin9, CEACAMI, SIRP alpha, DC-SIGN, CD200R, DR3, CDCHK1, CHK2, A2aR, or B-7 family proteins.

A checkpoint inhibitor can interact with a ligand of a checkpoint protein. For instance, by way of non-limiting example, a ligand of a checkpoint protein includes: PD-L1, PD-L2, ICOS ligand, VISTA, 4-1BBL, Herpesvirus Entry Mediator (HVEM), tumor necrosis factor receptor superfamily member 14 or TNFRSF14, MHC class I, MHC class II, PVR, OX-40L, CD70, CD40, GITRL, CD155, CD48, GAL9; HMGB1, CEASAM-1, phosphatidyl serine (PtdSer), IDO, TDO, CD47, BTN2A1, CD200, TL1A, CD112, CD155, MHCII, LSECtin, CHK1, CHK2, A2aR, or a B-7 family ligand (e.g., CD80 (B7-1), CD86 (B7-2), B7-H3, B7-H4, B7-H7 (HHLA2), etc.).

A checkpoint inhibitor can be an antagonist. For example, a checkpoint inhibitor can antagonize a checkpoint protein. A checkpoint inhibitor can be an antagonist to a ligand of a checkpoint protein. The antagonist can be a biological molecule such as a biological therapeutic. A checkpoint inhibitor can be an antibody or an antigen-binding portion thereof, such as a monoclonal antibody, a humanized antibody, a fully human antibody, a fusion protein, or a combination thereof. A checkpoint inhibitor can be a small molecule. A checkpoint inhibitor can be a rationally designed peptide. A checkpoint inhibitor can be a cell or cell preparation (e.g., cells that express a checkpoint inhibitor).

The checkpoint inhibitor can inhibit PD-1. Programmed cell death 1 (PD-1) is a key checkpoint receptor expressed by activated T and B cells, and mediates immunosuppression. Among other things, PD-1 limits activity of T cells in peripheral tissues during an inflammatory response to infection. In addition, as a checkpoint protein, PD-1 blockade can enhance T-cell proliferation and cytokine production in response to a challenge by specific antigen targets or by allogeneic cells in mixed lymphocyte reactions.

Without intending to be bound by theory, it is believed that blockade of PD-1, in combination with a LILRB2/1 antibody product as disclosed herein, relieves macrophage-mediated T cell suppression/exhaustion, increases T cell proliferation and cytokine production, and improves immune cell effector function. PD-1 blockade can be accomplished by a variety of mechanisms. For instance, PD-1 blockade can be achieved by blocking PD-1 binding to its ligands. PD-1 can be blocked with a checkpoint inhibitor that is a PD-1 antagonist. For instance, a PD-1 antagonist can be a PD-1 antibody (e.g., nivolumab, pembrolizumab, etc.). A PD-1 antagonist can be a small molecule [e.g., INCB-086550 (Incyte) or small molecules disclosed in, e.g., Wu et al., Acta Pharmacol Sin. 2021 42:1-9; Jiao et al., Curr Pharm Des. 2018 24(41):4911-20; and Liu et al., Cancer Cellnt. 2021 21(1):239]. A PD-1 antagonist can be or can comprise rationally designed peptide (e.g., APi2568). A PD-1 antagonist can be or can comprise a cell or cell preparation (e.g., cells that express a PD-1 binding agent, e.g., a PD-1 antibody, e.g., HerinCAR-PD1).

Exemplary PD-1 antibodies suitable for use in the methods include, without limitation, nivolumab (ONO-4538, BMS-936558, MDX1106, Opdivo®; Bristol-Myers Squibb), pembrolizumab (MK-3475, Keytruda®; Merck), cemipilimab (e.g., cemiplimab-rwlc (Libtayo™; Regeneron)), dostarlimab (e.g., dostarlimab-gxly (Jemperli™; GlaxoSmithKline)), pimivalimab (IgG4)(JTX-4014; Jounce Therapeutics), spartalizumab (IgG4)(PDR001; Novartis), camrelizumab (SHR1210; Jiangsu HengRui Medicine), sintilimab (IBI308; Innovent and Eli Lilly), tislelizumab (BGBA317; BeiGene), toripalimab (JS 001; Shanghai Junshi Bioscience), INCMGA00012 (MGA012; Incyte and MacroGenics), AMP-224 (PD-L2/Ig fusion; AstraZeneca and GlaxoSmithKline), AMP-514 (IgG4K) (MEDI0680; AstraZeneca), balstilimab (AGEN2034; Agenus), and/or a PD-1 binding domain of any of them.

Another exemplary PD-1 antagonist is a rationally designed peptide such as, e.g., APi2568, which comprises a B-cell epitope (amino acids 92-110 from PD-1) linked to a promiscuous T-cell epitope (amino acid residues 288-302 from measles virus fusion protein) via a 4-amino acid linker, and combined with Water for Injection (WFI) forms the drug product, IMU-201, which becomes PD1-Vaxx when emulsified with excipient Montanide ISA 720 VG.

Another exemplary PD-1 antagonist is a cell expressing a PD-1 antibody, for example, PD-1 antibody-expressing-CAR-T cells (e.g., HerinCAR-PD1 cells).

In some embodiments, the checkpoint inhibitor is a PD-L1 antagonist, such as a PD-L1 antibody. In some embodiments, the PD-L1 antibody is selected from the group consisting of avelumab, durvalumab, atezolizumab, envafolimab, cosibelimab, LY3300054 CA-170, BMS-936559, and PD-L1-binding fragments or combinations thereof. In some embodiments, the PD-L1 antagonist comprises AUNP-12, BMS-986189, a PD-L1 binding domain comprising CDRs of an antibody selected from the group consisting of avelumab, durvalumab, atezolizumab, envafolimab, cosibelimab (CK-301), LY3300054, CA-170, and BMS-936559, and active fragments thereof, or combinations thereof.

A checkpoint inhibitor can inhibit cytotoxic T-lymphocyte-associated protein 4 (CTLA-4) or a ligand thereof. CTLA-4 antibodies bind to CTLA-4 and block the interaction of CTLA-4 with its ligands CD80/CD86, which ligands are expressed on antigen presenting cells. Accordingly, a CTLA-4 inhibitor that blocks interaction of CTLA-4 and its ligands can block negative down regulation of the immune responses elicited by the interaction of these molecules. Thus, a checkpoint inhibitor can be a CTLA-4 antagonist such as described in U.S. Pat. Nos. 5,811,097; 5,811,097; 5,855,887; 6,051,227; 6,207,157; 6,682,736; 6,984,720; and 7,605,238. Moreover, exemplary CTLA-4 antibodies include: ipilimumab (10D1, MDX-D010, Yervoy™; Bristol-Myers Squibb), tremelimumab (ticilimumab, CP-675,206, Imjudo®; AstraZeneca), and quavonlimab (MK-1308; Merck). A CTLA-4 antagonist can comprise a CTLA-4-binding domain or fragment thereof of any CTLA-4 antagonist. A CTLA-4 antagonist can comprise a small molecule (see, e.g., Wang et al., Biochim Biophys Acta Rev Cancer. 2019 1871(2):199-224).

Lymphocyte-activation gene 3 (LAG-3, also known as CD223) is a CD4− related transmembrane protein that competitively binds MHC II and acts as a co-inhibitory checkpoint for T cell activation [e.g., Goldberg and Drake, Curr Top Microbiol Immunol. 2011 344:269-78]. A checkpoint inhibitor can be a LAG3 antagonist. A LAG3 antagonist can be a LAG-3-binding protein (e.g., an antibody) or a protein that binds to a LAG3 ligand. Non-limiting examples of LAG-3 antibodies include: LAG525 (IMP701, Novartis/Prima Biomed), MK-4280 (Merck Sharp & Dohme), REGN3767 (Regeneron Pharmaceuticals), relatlimab (BMS-986016, Bristol-Myers Squibb), and BI 754111 (Boehringer Ingelheim).

T cell immunoglobulin mucin 3 (TIM-3, also known as Hepatitis A virus cellular receptor (HAVCR2)) is a type I glycoprotein receptor that binds to S-type lectin galectin-9 (Gal-9). TIM-3, is a widely expressed ligand on lymphocytes, liver, small intestine, thymus, kidney, spleen, lung, muscle, reticulocytes, and brain tissue. Binding of Gal-9 by the TIM-3 receptor triggers downstream signaling to negatively regulate T cell survival and function. A checkpoint inhibitor can be an agent that inhibits TIM-3. A checkpoint inhibitor can be a TIM-3 antagonist, such as a TIM3 antibody or an antibody to a TIM-3 ligand. A TIM-3 antagonist can comprise a TIM-3-binding domain or fragment thereof of any TIM-3 antagonist. Non-limiting examples of TIM-3 antagonists include: TSR-022 (AnaptysBiolTesaro, Inc.) and MGB453 (Novartis). Additional exemplary TIM-3 binding proteins (e.g., antibodies) are known in the art and are disclosed, e.g., in U.S. Pat. Nos. 9,103,832, 8,552,156, 8,647,623, 8,841,418; U.S. Patent Pub. Nos. 2016/0200815, 2015/0284468, 2014/0134639, 2014/0044728, 2012/0189617, 2015/0086574, 2013/0022623; and PCT Pub. Nos. WO 2016/068802, WO 2016/068803, WO 2016/071448, WO2011/155607, and WO2013/006490.

T cell immunoglobulin and ITIM domain (TIGIT) is an inhibitory receptor expressed on lymphocytes. TIGIT interacts with CD155 expressed on antigen-presenting cells or tumor cells to down-regulate T cell and natural killer (NK) cell functions. A checkpoint inhibitor can be a TIGIT antagonist. A TIGIT antagonist can bind to TIGIT or to a TIGIT ligand. A TIGIT antagonist can be a TIGIT antibody or an antibody to a TIGIT ligand. A TIGIT antagonist comprises a TIGIT-binding domain or fragment thereof of any TIGIT antagonist. Non-limiting examples of TIGIT antagonists include: Tiragolumab (MTIG7192A; RG6058) (Genentech/Roche), AB154 (Arcus Bioscience), vibostolimab (MK-7684) (Merck), BMS-985207 (Bristol-Myers Squibb), ASP8374 (Astellas Pharma; Potenza Therapeutics), and ASP8374 (Astellas Pharma; Potenza Therapeutics).

Exemplary anti-CD27 agonists include MK-5890 (Merck).

Exemplary ICOS antibodies include vopratelimab (JTX-2011; Jounce).

In some embodiments, the LILRB2/1 antibody product and an immune checkpoint inhibitor are co-formulated. In some embodiments, the LILRB2/1 antibody product and the immune checkpoint inhibitor are in separate formulations. In some embodiments, the LILRB2/1 antibody is administered with a coformulation of a PD-1 antagonist, such as a PD-1 antibody, and a CTLA-4 antagonist, such as a CTLA-4 antibody, with the dosages of each component controlled to provide a safe and effective treatment for the subject. In some embodiments, the LILRB2/1 antibody is administered with a coformulation of pembrolizumab/quavonlimab (MK-1308A; Merck).

Also provided are methods of treating cancer comprising administering a therapeutically effective amount of a LILRB2/1 antibody product provided herein and a colony-stimulating factor 1 (CSF1) antagonist. Colony-stimulating factor 1 receptor (CSF1R) inhibitors are being developed for cancer therapy. Canarile et al., J Immunother Cancer. 2017 5(a):53. For example, pexidartinib (PLX-3397), has been shown to alter the distribution of tumor-associated macrophages in the tumor microenvironment and promote enrichment of macrophages having an M1-like phenotype. A CSF1 antagonist can be a CSF1 antibody or a CSF1R inhibitor. Such CSF1 antagonists include, for example, pexidartinib, PLX7486, ARRY-382, JNJ-40346527, BLZ945, emactuzumab, AMG820, IMC-CS4, MCS110, PD-0360324, and cabiralizumab.

Also provided are methods of treating cancer comprising administering a therapeutically effective amount of a LILRB2/1 antibody product provided herein and an agonistic CD40 antibody.

Also provided are methods of treating cancer comprising administering a therapeutically effective amount of a LILRB2/1 antibody product provided herein and an inhibitory CD47 antibody.

Also provided are methods of treating cancer comprising administering a therapeutically effective amount of a LILRB2/1 antibody product provided herein and an effective amount of a class IIa histone deacetylase (HDAC) inhibitor, e.g., TMP195.

Also provided are methods of treating cancer comprising administering a therapeutically effective amount of a LILRB2/1 antibody product provided herein and a TLR7 or TLR8 agonist. The toll-like receptors TLR7 and TLR8 appear to be involved in macrophage polarization in the tumor microenvironment. Agonists of either or both of these receptors can promote functional orientation of tumor-associated macrophages toward M1-like phenotype. It has been reported that resiquimod-loaded β-cyclodextrin nanoparticles have an anti-tumoral effect, which can be enhanced in the presence of a PD-1 antagonist. Rodell et al., Nat Biomed Eng. 2018 2:578-88. Such TLR7/TLR8 agonists include, for example, resiquimod (B848), motolimod (VTX-2337), and imiquimod.

Methods of treating a disease condition in a subject, in which the disease condition is characterized by or mediated through LILRB2 expression by myeloid cells, are also provided. The myeloid cell can be macrophages. The myeloid cells can be osteoclasts or osteoclast precursors. The methods comprise administering to the subject a therapeutically effective amount of an LILRB2/1 antibody product disclosed herein.

Methods of treatment or prophylaxis of a bone-metabolism disorder in a subject are also provided. The bone-metabolism disorder can be osteoporosis, bone destruction accompanying rheumatoid arthritis, cancerous hypercalcemia, bone destruction accompanying multiple myeloma or cancer metastasis to bone, giant cell tumor, osteopenia, tooth loss due to periodontitis, osteolysis around a prosthetic joint, bone destruction in chronic osteomyelitis, bone Paget's disease, renal osteodystrophy, or osteogenesis imperfecta. The bone metabolism disorder can be osteoporosis. The osteoporosis can be postmenopausal osteoporosis, senile osteoporosis, secondary osteoporosis due to the use of a therapeutic agent such as a steroid or an immunosuppressant, or osteoporosis accompanying rheumatoid arthritis.

### Other Terminology

As used herein, singular forms “a,” “and,” and “the” include plural referents unless the context clearly indicates otherwise. Thus, for example, reference to “an antibody” includes multiple antibodies.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000-fold includes 1-, 2-, 3-, 4-, 5-, 6-, 7-, 8-, 9-, 10-, 11-, 12-, 13-, 14-, 15-, 16-, 17-, 18-, 19-, or 20-fold, etc., as well as 1.1-, 1.2-, 1.3-, 1.4-, or 1.5-fold, etc., 2.1-, 2.2-, 2.3-, 2.4-, or 2.5-fold, etc., and so forth.

“About” a number, as used herein, refers to range including the number and ranging from 10% below that number to 10% above that number. “About” a range refers to 10% below the lower limit of the range, spanning to 10% above the upper limit of the range.

As used herein, “contemplated,” “can,” “can be,” “may” and “may be” all indicate something contemplated by the inventors that is functional and available as part of the subject matter provided.

### EXAMPLES

While the following examples describe specific embodiments, variations and modifications will occur to those skilled in the art. Accordingly, only such limitations as appear in the claims should be placed on the invention.

Antibodies binding to both LILRB2 and LILRB1 were discovered from rabbit B cells immunized with human LILRB2 protein. B cells from immunized rabbits were cultured at clonal density, and IgG antibodies in supernatants were evaluated for binding to human and LILRB1 and LILRB2 proteins by enzyme-linked immunosorbent assay (ELISA).

Top LILRB2/1 clones were identified based on activity in a panel of functional and phenotypic assays using primary human macrophages and T cells. Variable-regions from positive hits were sequenced, cloned, and expressed as recombinant rabbit/human IgG1 and IgG4 Fc chimeras.

Select clones were humanized by in silico methods.

### Example 1: Immunization, Cloning and Initial Screening

Dual LILRB2/1 antibodies were discovered from B cells derived from rabbits immunized with human LILRB2 protein. Briefly, two female New Zealand White rabbits were immunized with purified human LILRB2 extracellular domain (ECD)—rabbit Fc fusion protein (SEQ ID NO: 43) (OncoResponse), using a standard immunization method. Rabbits were given a boost each on day 21, day 42, and day 73 following primary immunizations. The pre-immunization bleeds and test bleeds were evaluated for specific antibody titer by indirect ELISA. On day 83 following primary immunization, heparinized whole blood was collected for rabbit monoclonal antibody development. B cells in peripheral blood from both rabbits were collected after last boost, then isolated, purified, and cultured at clonal density using proprietary methods. Bio-panning was performed using human LILRB2 ECD—human Fc fusion protein (SEQ ID NO: 44). B cell culture supernatants from 80 96-well plates were transferred to ELISA plates coated with the human-Fc-LILRB2 ECD fusion protein. An indirect ELISA was performed probing with secondary anti-rabbit IgG antibody (ImmunoPrecise, Antibodies Ltd.). For the recovery of antibodies specific to target antigen, negative screening of the positive candidates was performed against an unrelated human-Fc fusion protein (ImmunoPrecise). B cell supernatants were also evaluated for ability to block LILRB2-Fc (R&D Systems, No. 2078-T4) and LILRB1-Fc (R&D Systems, No. 2017-T2-050) binding to HLA-G. The top 96 responding wells were preserved in a standard RNA lysis buffer for antibody RNA isolation and generation of recombinant plasmid DNA.

The rabbit antibody heavy and light (kappa) chain variable domains of the top-ranked positive clones were cloned into separate mammalian expression vectors containing the human IgG4 heavy and kappa constant domains. Recombinant monoclonal antibodies (rabbit-human chimeras) were generated using transfected CHO-K1 cells, purified using standard methods and evaluated in several biochemical and cell-based functional assays.

### Example 2: Antibody Humanization

The rabbit/human chimeric clone designated B21A4, an IgG4 clone among those identified as described in Example 1, was selected for humanization. Humanization was conducted in silico using a proprietary methodology (Fusion Antibodies, Belfast, Northern Ireland). In this system, a model of the parental variable domains is generated to enable structure-guided humanization. Sequences are aligned to a panel of human germline sequences selected for preferential manufacturability properties, and the non-human amino acids are grafted onto the human sequence using a proprietary CDRx™ humanization platform. The first round of humanization conducted for the clone met the EC₅₀ criteria, so only one round of humanization was performed.

Five humanized heavy and five light variable domain chains were generated for Clone B21A4. Humanized variants are generally referred to herein as B21H variants.

The humanized amino acid sequences were submitted to GenScript. GenScript reverse translated and codon optimized each variable region sequence for mammalian cell expression using their proprietary methodology. The genes encoding the signal sequence plus variable domains were synthesized at GenScript and cloned into the pTT5 vector in-frame with a human constant IgG4 domain for the heavy chain and a human kappa constant domain for the light chain. The resulting plasmid vectors, one for the light chain and one for the heavy chain, were transiently co-transfected into HEK293-6E cells (National Research Council Canada), and conditioned media were harvested seven days later. Thus, 25 IgG4 variants were expressed in mammalian cell culture as a combinatorial library of the five light and five heavy chains. These IgG4 antibodies are designated in the format “B21H4-XY”, where “B21” refers to the parent clone, “H” signifies humanized, “4” signifies IgG4 isotype, and the suffix -XY distinguishes among VH-VL combinations, where X and Y each vary from 1 to 5, corresponding to the five humanized VH variants (VH1-VH5) and five humanized VL variants (VL1-VL5) set forth in Table 3. Accordingly, these IgG4 variant antibodies are designated B21H4-11 through B21H4-55. In addition, the B21A4 chimera was included as a transfection control. The transfection supernatant concentrations of the 25 humanized IgG4 variants and the parental clone B21A4 chimeric antibody were determined by an IgG4 specific AlphaLISA® immunoassay (PerkinElmer, No. AL310C) (Table 4).

These transfection supernatants were then used to generate binding data for the B21H variants via ELISA using MSD (Meso Scale Diagnostics) instrumentation (Table 4). Success was defined as achieving a binding constant within two-fold of the parental mAb, and in this case, the EC₅₀ was determined by ELISA using a rabbit variable region fused to a human IgG4 constant region as parental chimeric mAbs. Based on the results of the binding data using transfection supernatants, the 25 B21H IgG4 antibodies were winnowed to 15 antibodies for further characterization. These 15 B21H IgG4 antibodies were expressed and purified by affinity chromatography and again assayed for binding.

All five humanized heavy chains were then converted into IgG1 isotypes by using restriction sites common to vectors encoding either the human IgG4 or IgG1 constant regions. These common restriction sites between IgG4 and IgG1 vectors allow in-frame fusion of the variable regions to the constant region. The same set of select 15 humanized IgG4 variants, as well as the original B21A4 chimera, were re-expressed as human IgG1 isotypes and tested for binding (Example 3 and Table 5). The designations of these IgG1 variants follow those for the IgG4 variants above, in the format “B21H1-XY”, where the prefix B21H1 signifies IgG1, while the suffix -XY is employed to identify the same five heavy and 5 light chain variants. Accordingly, the 25 IgG1 variant antibodies are designated B21H1-11 through B21H1-55.

### Example 3: Binding of Antibodies to LILRB1 and LILRB2 (Human and Cynomolqus) by ELISA

To confirm binding to human LILRB1 and human LILRB2, transfection supernatants (obtained in Example 2) were evaluated for binding by ELISA. The conditioned media from these transient transfections were assayed for human IgG concentration by AlphaLISA® immunoassay (PerkinElmer, No. AL310C) (Table 4). A plate-based MSD ELISA was performed in which human LILRB1-His or LILRB2-His was immobilized, and binding of transfection supernatants of humanized mAb variants was detected via an anti-human IgG Sulfo-tag-labeled secondary antibody. The IgG1 version of the parental rabbit/human chimera (designated B21A1) transfection served as the benchmark EC₅₀ positive control. Antibody titers and EC₅₀ values are shown in Table 4.

Recombinant human and cynomolgus LILRB1 proteins (R&D Systems, No. 8989-T2-050; ACROBiosystems, No. CDJ-C52H3) and recombinant human and cynomolgus LILRB2 proteins (ACROBiosystems, No. LI2-H5220; No. L12-C52H3) were diluted in PBS to 4 μg/mL and added to a multi-array 384-well high-binding plates (MSD, No. L21XA4) at 10 μL/well and incubated at 4° C. overnight. The plates were washed several times with Wash Buffer (0.05% Tween® 20 in PBS), using a microplate washer and then blocked with 70 μL/well of 5% BSA in PBS for 1 hour (h) at room temperature (RT) with shaking at 700 rpm. After blocking, 20 μL/well of anti-LILRB2/LILRB1 antibodies or isotype control were added to the plates, and the plates were sealed and incubated for 2 h at RT with shaking at 700 rpm. After primary antibody binding, plates were washed five times with 70 μL/well of Wash Buffer (0.05% Tween® 20 in PBS) using a microplate washer. Secondary detection antibody (SULFO-TAG™-labeled goat anti-human IgG antibody (MSD, No. R32AJ-1)) was diluted to 4 μg/mL in assay diluent (1% BSA in PBS) and 10 μL/well was added to plates and incubated at RT protected from light for 1 h, with shaking at 700 rpm. Following incubation, plates were washed five times with Wash Buffer, using a microplate washer. After removal of the final wash, 35 μL/well of MSD Gold Read Buffer B (MSD, No. R60AM-4) was added to all wells using a Multidrop combo washer/dispenser, and plates were read using MESO Sector 600MM instrument (MSD) within 5 minutes (min) after addition of Read Buffer. Signals as counts per minute (cpm) were recorded. EC₅₀ values were calculated using GraphPad Prism based on log concentration of the primary antibodies versus cpm values.

TABLE 4
Antibody humanization: Humanized IgG4 variants have
similar or better binding to LILRB1 and LILRB2 by
ELISA compared to B21A4 chimera. N.T. = not tested.

| B21H | Heavy | Light | | Human | Human |
|---|---|---|---|---|---|
| Variant VH | Chain | Chain | mAb | LILRB1- | LILRB2- |
| and VL | SEQ ID | SEQ ID | titer | His EC₅₀ | His EC₅₀ |
| Domains | NO: | NO: | (μg/mL) | (ng/mL) | (ng/mL) |
| B21H4-11 | 28 | 38 | 43.3 | 2.24 | 1.45 |
| B21H4-12 | 28 | 39 | 10.9 | 2.41 | 0.74 |
| B21H4-13 | 28 | 40 | 7.3 | 1.50 | 1.01 |
| B21H4-14 | 28 | 41 | 45.1 | 1.30 | 1.46 |
| B21H4-15 | 28 | 42 | 13.7 | 3.10 | 1.25 |
| B21H4-21 | 29 | 38 | 8.8 | 2.32 | 1.93 |
| B21H4-22 | 29 | 39 | 3.7 | 1.85 | 1.06 |
| B21H4-23 | 29 | 40 | 10.8 | 2.85 | 1.08 |
| B21H4-24 | 29 | 41 | 7.7 | 1.95 | 0.84 |
| B21H4-25 | 29 | 42 | 28.8 | 3.95 | 1.42 |
| B21H4-31 | 30 | 38 | 21.4 | 3.58 | 2.02 |
| B21H4-32 | 30 | 39 | 13.1 | 2.46 | 0.95 |
| B21H4-33 | 30 | 40 | 10.5 | 2.66 | 0.72 |
| B21H4-34 | 30 | 41 | N.T. | N.T. | N.T. |
| B21H4-35 | 30 | 42 | 26.4 | 3.91 | 1.73 |
| B21H4-41 | 31 | 38 | 44.3 | 5.71 | 1.38 |
| B21H4-42 | 31 | 39 | 42.9 | 3.12 | 1.08 |
| B21H4-43 | 31 | 40 | 10.0 | 3.04 | 0.63 |
| B21H4-44 | 31 | 41 | 54.1 | 3.23 | 1.63 |
| B21H4-45 | 31 | 42 | 28.3 | 4.70 | 0.26 |
| B21H4-51 | 32 | 38 | 39.8 | 3.88 | 2.51 |
| B21H4-52 | 32 | 39 | N.T. | N.T. | N.T. |
| B21H4-53 | 32 | 40 | 16.0 | 4.70 | 1.67 |
| B21H4-54 | 32 | 41 | 104.0 | 4.85 | 2.75 |
| B21H4-55 | 32 | 42 | 19.9 | 2.92 | 1.57 |
| B21A4 | 4 | 5 | 14.7 | 3.73 | 2.63 |

Binding of 15 purified humanized IgG1 antibodies to human and cynomolgus LILRB1 and human LILRB2 is demonstrated in Table 5, using the MSD ELISA method described above. All antibodies showed specific binding to human LILRB1 and LILRB2, and weaker binding to cynomolgus LILRB1. Only minimal binding of the antibodies to cynomolgus LILRB2 was observed (data not shown). Table 5 presents EC₅₀ values for the MSD ELISA binding studies with purified antibodies. N.T.=not tested.

TABLE 5
ELISA EC₅₀ of humanized LILRB2/1 antibodies (IgG1)

| | Heavy | Light | Human | Cynomolgus | Human |
|---|---|---|---|---|---|
| | Chain | Chain | LILRB1 | LILRB1 | LILRB2 |
| | SEQ ID | SEQ ID | EC₅₀ | EC₅₀ | EC₅₀ |
| Antibody | NO: | NO: | (ng/mL) | (ng/mL) | (ng/mL) |
| B21A1 | 4 | 5 | 10.5 | 306.5 | 38.02 |
| B21H1-45 | 36 | 42 | 13.39 | 284 | 46.14 |
| B21H1-43 | 36 | 40 | 7.34 | 1508 | 24.79 |
| B21H1-33 | 35 | 40 | 6.16 | 5852 | 22.48 |
| B21H1-12 | 33 | 39 | 6.14 | N.T. | 17.43 |
| B21H1-24 | 34 | 41 | 8.64 | 285.7 | 28.7 |
| B21H1-32 | 35 | 39 | 6.46 | 271.5 | 21.87 |
| B21H1-13 | 33 | 40 | 5.45 | N.T. | 20.32 |
| B21H1-22 | 34 | 39 | 8.18 | N.T. | 25.3 |
| B21H1-23 | 34 | 40 | 8.64 | N.T. | 30.66 |
| B21H1-42 | 36 | 39 | 9.19 | N.T. | 28.44 |
| B21H1-15 | 33 | 42 | 5.63 | N.T. | 21.55 |
| B21H1-25 | 34 | 42 | 9.5 | 377.4 | 31.82 |
| B21H1-11 | 33 | 38 | 7.34 | 4402 | 30.62 |
| B21H1-14 | 33 | 41 | 5.93 | 450 | 18.69 |
| B21H1-55 | 37 | 42 | 9.44 | 250.3 | 36.87 |

### Example 4: Affinity of LILRB2/1 Antibodies by Bio-Layer Interferometry (BLI)

Affinity measurements of human LILRB1 and LILRB2 binding to immobilized humanized LILRB2/1 antibody variants was determined by BLI using a 16-channel Octet® RH16 instrument using anti-human IgG Fc capture (ACH2) biosensors (Sartorius) and compared to the IgG1 form of the parent clone, B21A1. Kinetic Buffer was prepared using PBS, pH 7.4 (Bulldog Bio, No. 2N18501) containing 0.1% BSA (Fisher Scientific, No. BP9704-100), 0.05% Tween®-20 (Fisher Scientific, No. BP337-500) and 0.02% sodium azide (NaN₃; RICCA Chemical, No. 7144.8-32). The Kinetic Buffer was filtered through a 0.2 μm filter unit (Thermo Scientific, No. 5690020) before use. All antibodies and analytes (LILRB2 His: Acro Biosystems, No. LI2-H5220; LILRB1 His: R&D Systems, No. 8989-T2-050) were prepared in Kinetic Buffer. A 96-well, black, flat-bottom microplate (Greiner Bio-One, No. 655209) was used to set up the experiment. The biosensor tips were hydrated in Kinetic Buffer for at least 10 min before the start of the assay. Initial baseline in Kinetic Buffer was run for 60 seconds (s). LILRB2/1 antibodies at 4 μg/mL were loaded onto the ACH2 biosensors for 45 s. Following a baseline for 60 s in Kinetic Buffer, the loaded biosensors were exposed to a series of analyte (LILRB1 or LILRB2) concentrations (0-40 nM) for 300 s for the association step. During disassociation, the biosensors were dipped in Kinetic Buffer for 300 s. The regeneration step was performed between each antibody/analyte change by dipping into glycine pH 1.5 buffer (Cytiva, No. BR100354), followed by a neutralization step into Kinetic Buffer for 5 s each and in triplicate, respectively. Background subtraction was used to correct for any sensor drift. All experiments were performed with shaking at 1,000 rpm. Background wavelength shifts were measured from reference biosensors that were loaded only with antibody. Octet® Analysis Studio 12.2 software was used to fit the data to a 1:1 binding model along with Savitzky-Golay filtering to extract association and disassociation rates. The K_{D} was calculated using the ratio K_{off}/Kₒₙ. Kinetic constant ranges for RH16 system are between 1 mM to 1 pM. Any affinity higher than 1 pM will be shown as K_{D}<1 pM. All experiments were performed in triplicate and as independent experiments. The tested humanized LILRB2/1 antibodies have K_{D} values similar to that of the parent B21A1, with K_{D} between 1.3-4.2 pM for LILRB1 protein, and K_{D}<1 pM for LILRB2 protein.

### Example 5: Preparation of Cells Expressing Human LILRB2

Cells stably expressing human LILRB2 were generated using human embryonic kidney cells (HEK293T/17; ATCC, No. CRL-11268), and were cultured per ATCC guidelines in DMEM (Gibco, No. 11965-084) supplemented with 10% FBS (HyClone, No. SH30396.03; heat-inactivated before use). Pre-packaged lentiviral particles containing puromycin-selectable lentivector backbone were purchased from G&P Biosciences: without the gene of interest (negative control; No. LTV0001) with human LILRB1 (No. LTV2991) or with human LILRB2 (No. LTV2992). HEK293T cells (5×10⁴) were transduced with human LILRB2 lentiviral particles at multiplicity of infection (MOI) of 10, in the presence of 8 μg/mL polybrene infection/transfection reagent (Millipore, No. TR-1003-G), for 24 h at 37° C., 5% CO₂. Virus-containing medium was then removed, and cells were allowed to recover in fresh medium for two days prior to selection in media containing 0.75 μg/mL of puromycin (Thermo Fisher, No. A1113803). By selecting for stable LILRB2 expression based on puromycin as a selection marker, populations of LILRB2-expressing cells were isolated and cultured. Expression of LILRB2 on puromycin-resistant cell populations was confirmed by flow cytometry using BD FACSymphony™ cytometer and LILRB2-specific antibodies. Alongside the LILRB2 transductions, cells were also transduced with negative control lentivirus particles, using the same protocol, to generate a stable, puromycin-resistant control cell line (contains lentivector backbone without the gene insert) for use in assays.

Once target expression was confirmed, the cells were expanded. The selected polyclonal pool of cells was banked at 4-5 million cells/mL/vial and master stocks were made. Banking of cells involves pelleting cells from culture by centrifugation, removing the medium, and resuspending cells in 4° C. DMEM+10% FBS first, then adding an equal volume of 2×ATCC recommended freezing medium (DMEM+10% FBS plus 10% DMSO) such that cells were at a density of 4-5 million/mL in 1× freezing medium (DMEM+10% FBS plus 5% DMSO). Aliquots of the cell suspension were placed into a 4° C. Mr. Frosty™ alcohol-based slow freezing system and then immediately stored at −80° C. These frozen cell aliquots were stored at −80° C. for one day and then were moved to liquid nitrogen vapor phase tank.

For use in assays, a master stock vial was thawed in a 37° C. water bath for 1 min and centrifuged to pellet cells. Cells were suspended in DMEM+10% FBS and expression of target protein was checked by flow cytometry. These cells were expanded in culture to make 30 working stocks and expression of target protein was checked by flow cytometry. These cells were expanded in culture to make 30 working stocks.

### Example 6: Dual LILRB2/1 Antibodies Bind to Cells Expressing Human LILRB1 and LILRB2

While LILRB2 expression is mostly restricted to myeloid cells, LILRB1 has a broader expression on immune cell subsets, including B cells, T cells and NK cells. To confirm binding of LILRB2/1 antibodies to cells expressing these receptors, flow cytometry binding assays were performed using HEK293T cells modified to stably express human LILRB2 (Example 5), or the human B cell lymphoma cell line 721.221, which natively expresses human LILRB1. The 721.221 wild type immortalized cancer cell line, a B cell lymphoma line, was used to characterize the LILRB1 binding of anti-LILRB2/1 antibodies.

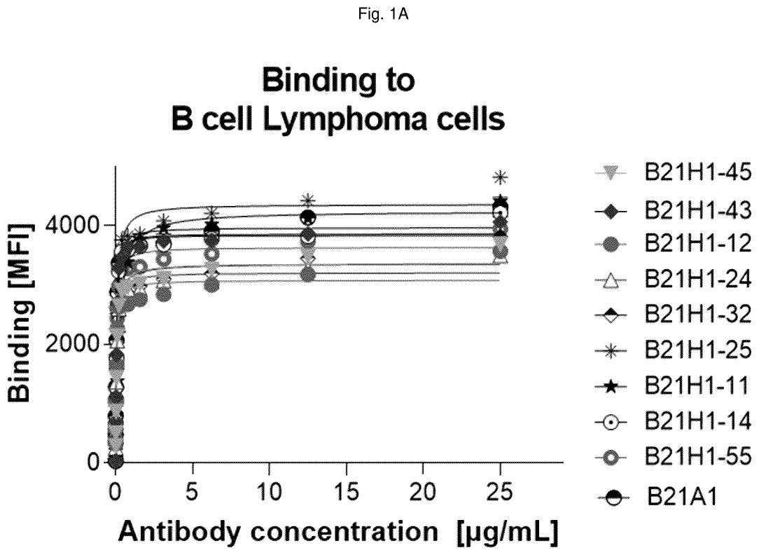

Frozen cells were removed from storage in vapor phase liquid nitrogen and thawed by gentle swirling in a water bath at 37° C., then resuspended in DMEM media with 10% FBS. Cells were placed in a 15 mL conical tube and centrifuged at 300×g for 5 min, then supernatant was removed. Cells were resuspended in DMEM+10% FBS at a cell density of 2×10⁵ cells/mL in a T75 flask for culturing, and incubated at 37° C., 5% CO₂ for 3 days before passaging. Cells were passaged when the cell density reached 1×10⁶ cell/mL. On the day of the assay, cells were centrifuged, then supernatant was removed. Cells were resuspended in Blocking Buffer B (FACS Buffer containing 10% FBS and 0.5 mg/mL human IgG1 (Athens Research, No. 16-16-090707-1M)+0.05% NaN₃, 5% (w/v)) in a 15 mL conical tube, then incubated at 4° C. for 30 min. Cells in 25 μL Blocking Buffer B were transferred to a 384-well plate at 2.5×10⁴ cells/well and 25 μL of titrated AF647-conjugated LILRB2/1 antibodies or AF647-conjugated IgG1 isotype control were added directly to each well at 2× final assay concentration. Cells were incubated with antibodies at 4° C. for 1 h. Cells were washed twice in FACS Buffer (PBS containing 2 mM EDTA+1% FBS+0.05% NaN₃) and then stained with for 10 min with Zombie Violet™ fixable viability dye (BioLegend, No. 423114) at RT, washed with FACS Buffer and resuspend in 75 μL of FACS Buffer for acquisition on a BD FACSymphony™ or BD FACSCanto™ II flow cytometer (BD Biosciences), as described in Example 6. The mean fluorescence intensity (MFI) binding of LILRB2/1 antibodies was gated on live cells using FlowJO software (10.5.3, FlowJO, LLC) and GraphPad prism for EC₅₀ binding calculations (Table 6). The LILRB2/1 antibodies bound LILRB2-expressing HEK293T cells in a dose-dependent manner, with EC₅₀ values of 0.7-2.2 ng/mL. The LILRB2/1 antibodies bound 721.221 B cell lymphoma cells with EC₅₀ values of 0.042-0.2 μg/mL. Binding curves of antibodies to wild type 721.221 cells (FIG. 1A) and HEK293T-LILRB2 (FIG. 1B). In FIG. 1B, an anti-LILRB1 antibody (Comp 1; VH SEQ ID NO: 43 and VL SEQ ID NO: 44) is included as a negative control, showing minimal binding to LILRB2-HEK293T cells, and an anti-LILRB2 antibody (Comp 2; VH SEQ ID NO: 45 and VL SEQ ID NO: 46) is included as a positive control, showing binding to that of the tested LILRB2/1 antibodies. N.T.=not tested

TABLE 6
EC⁵⁰ values of IgG1 antibody binding to LILRB1-expressing
721.221 cells and LILRB2-expressing HEK293T cells.

| | 721.221 B cell | |
|---|---|---|
| | lymphoma | LILRB2-HEK293T |
| Antibody | EC₅₀ (ng/mL) | EC₅₀ (ng/mL) |
| B21A1 | 44.8 | 0.8 |
| B21H1-45 | 61.6 | 0.86 |
| B21H1-43 | 51.5 | 1.35 |
| B21H1-33 | N.T. | 1.44 |
| B21H1-12 | 41.7 | 1.02 |
| B21H1-24 | 59.5 | 1.42 |
| B21H1-32 | 51.8 | 1.23 |
| B21H1-13 | N.T. | 2.23 |
| B21H1-22 | N.T. | 1.07 |
| B21H1-23 | N.T. | 1.63 |
| B21H1-42 | N.T. | 1.25 |
| B21H1-15 | N.T. | 0.96 |
| B21H1-25 | 78.2 | 0.9 |
| B21H1-11 | 202.7 | 1.07 |
| B21H1-14 | 55.2 | 1.1 |
| B21H1-55 | 50.7 | 0.71 |

### Example 7: LILRB2/1 Antibodies Do Not Bind to Other LILRB or LILRA Family Members

To determine that the parent chimera anti LILRB2/1 antibody specifically binds to LILRB2 and LILRB1 and not to other LILRB and LILRA family members, a cross-reactivity assay was performed. Cross-reactivity was assessed by antibody binding to transiently transfected HEK293-6E cells by flow cytometry. Binding was evaluated to HEK293-6E cells transiently transfected with human LILRB-1, -2, -3, -4, and 5 plasmids (Origene, Nos. RC219949, RC217935, RC211228, RC220932, RC206516) or to cells transiently transfected with human LILRA-1, -2, -4, -5, and -6 plasmids (Origene, Nos. RC210808, RC205626, RC220452, RC212310 RC212965). AF647 mouse anti-human IgG-Fc secondary antibody (Jackson ImmunoResearch, No. 109-605-098) was used for the detection of bound human anti-LILRB2/1 antibodies on cells. Murine primary antibodies to LILRA and LILRB targets and corresponding isotype controls were used to confirm specific binding of all targets tested (R&D systems) while an AF647 F(ab′)₂ fragment goat anti-mouse antibody (Jackson ImmunoResearch, No. 115-606-062) was used for the detection of bound positive control antibodies. Binding of parent B21A1 and humanized IgG1 variants to other LILRA and LILRB family members is presented in Tables 7 and 8. At a concentration of 1 μg/mL, no binding of B21A1 or the humanized variants to LILRB-3, -4, or -5 was observed (Table 7). Additionally, no binding to LILRA-1, -2, -4, -5, or -6 was observed, confirming the specificity of B21A1 and its humanized variants to LILRB1 and LILRB2 (Table 8).

TABLE 7
Cross Reactivity of B21A1 and Humanized IgG1
Variants to other LILRA Family Members

| | hLILRA1 | hLILRA2 | hLILRA4 | hLILRA5 | hLILRA6 |
|---|---|---|---|---|---|
| mAb | (MFI) | (MFI) | (MFI) | (MFI) | (MFI) |
| B21H1-45 | 264 | 26.2 | 108 | 71 | 135 |
| B21H1-43 | 220.5 | 25.2 | 114 | 71.8 | 141 |
| B21H1-33 | 203.5 | 25.9 | 112 | 73.1 | 139.5 |
| B21H1-12 | 275.5 | 26.55 | 111.9 | 72.75 | 151 |
| B21H1-24 | 245.5 | 25.65 | 125 | 74.5 | 141 |
| B21H1-32 | 249 | 27.75 | 126 | 74.05 | 142 |
| B21H1-13 | 201 | 25.8 | 129.5 | 75.7 | 141.5 |
| B21H1-22 | 295 | 27.65 | 132 | 76.7 | 139.5 |
| B21H1-23 | 209.5 | 27.5 | 135.5 | 75.35 | 140.5 |
| B21H1-42 | 345 | 27.05 | 133.5 | 74.6 | 147 |
| B21H1-15 | 290.5 | 28.9 | 136.5 | 77.85 | 147 |
| B21H1-25 | 280 | 27.4 | 138.5 | 78.2 | 148.5 |
| B21H1-11 | 244.5 | 27.75 | 140 | 76.05 | 152.5 |
| B21H1-14 | 249 | 28.65 | 140.5 | 78.35 | 151 |
| B21H1-55 | 332.5 | 27.85 | 144.5 | 77.4 | 148 |
| B21A1 | 264 | 30.2 | 143 | 78.45 | 149.5 |
| hIgG1 | 253.5 | 34.65 | 166 | 82.7 | 197.5 |
| hIgG4 | 229 | 36.95 | 153.5 | 86.55 | 195.5 |
| αLILRA1 | 1602 | | | | |
| [mIgG2B] | | | | | |
| αLILRA2 | | 1743.5 | | | |
| [mIgG1] | | | | | |
| αLILRA4 | | | 1756 | | |
| [mIgG2A] | | | | | |
| αLILRA5 | | | | 1593.5 | |
| [mIgG2A] | | | | | |
| αLILRA6 | | | | | 2455.5 |
| [mIgG1] | | | | | |
| mIgG2A | | | 129.5 | 79.9 | |
| mIgG2B | 95 | | | | |
| mIgG1 | | 27.05 | | | 131 |

TABLE 8
Cross Reactivity of B21A1 (Chimera IgG1) and Humanized
IgG1 Variants to other LILRB Family Members

| | hLILRB1 | hLILRB2 | hLILRB3 | hLILRB4 | hLILRB5 |
|---|---|---|---|---|---|
| mAb | (MFI) | (MFI) | (MFI) | (MFI) | (MFI) |
| B21H1-45 | 997 | 6677 | 81 | 27.25 | 83.6 |
| B21H1-43 | 1125 | 6368.5 | 79.25 | 26.5 | 85.45 |
| B21H1-33 | 859.5 | 6294.5 | 89.25 | 27.35 | 85.85 |
| B21H1-12 | 963 | 6208 | 82.2 | 27.3 | 87.8 |
| B21H1-24 | 985 | 6045.5 | 86.25 | 28.65 | 87.05 |
| B21H1-32 | 1042.5 | 5458.5 | 99.2 | 28.75 | 87.9 |
| B21H1-13 | 1002.5 | 5368.5 | 84.7 | 29.1 | 89.85 |
| B21H1-22 | 914.5 | 5591 | 90.1 | 29.9 | 91.4 |
| B21H1-23 | 1055.5 | 5428 | 96.15 | 30.1 | 92 |
| B21H1-42 | 1160 | 5633.5 | 97 | 30.85 | 92.8 |
| B21H1-15 | 1135 | 5705 | 98.65 | 29.9 | 93.85 |
| B21H1-25 | 990.5 | 5324 | 93.9 | 32.4 | 90.6 |
| B21H1-11 | 999.5 | 5085.5 | 102.35 | 32.4 | 93.4 |
| B21H1-14 | 988 | 5762.5 | 104.5 | 33.5 | 93.3 |
| B21H1-55 | 1031 | 4121.5 | 108.5 | 33.45 | 94.8 |
| B21A1 | 940 | 5383 | 107 | 35.25 | 100.95 |
| hIgG1 | 253.5 | 40.5 | 123.5 | 40.15 | 133 |
| hIgG4 | 112.1 | 40.6 | 109 | 46 | 146 |
| αLILRB1 | 1224.5 | | | | |
| [mIgG2B] | | | | | |
| αLILRB2 | | 5750 | | | |
| [hIgG1] | | | | | |
| αLILRB3 | | | 732 | | |
| [mIgG2A] | | | | | |
| αLILRB4 | | | | 2724 | |
| [mIgG2A] | | | | | |
| αLILRB5 | | | | | 2462 |
| [mIgG2B] | | | | | |
| mIgG2A | | | 75.35 | 30.5 | |
| mIgG2B | 30.5 | | | | 76.75 |

### Example 8: LILRB2/1 Antibodies Block LILRB2-Fc and LILRB1-Fc Binding to HLA-G Expressed on Tumor Cells

Binding of LILRB1 or LILRB2 on macrophages to HLA-G on cancer cells enhances immunosuppressive functions of myeloid cells. The ability of the LILRB2/1 antibodies to block HLA-G binding on cancer cells is thought to be crucial to the clinical efficacy of anti-LILRB2 antibodies. LILRB2/1 antibodies were evaluated for blocking the binding of recombinant LILRB1-Fc or LILRB2-Fc protein to HLA-G expressed on 721.221 B lymphoma cells via flow cytometry.

Recombinant human LILRB1-Fc-Avitag protein (ACRO Biosystems, No. CDJ-H82F7) or LILRB2-Fc-Avitag protein (ACRO Biosystems, No. L12-H82F5) were diluted to 7.5 μg/mL in FACS Buffer and combined in equal parts volume with anti-LILRB2/1 antibodies at concentrations of 40, 13, 4, or 1.5 μg/mL (for LILRB1-Fc protein) and a final concentration of 15, 7.5, 3.75 and 1.88 μg/mL (for LILRB2-Fc protein), then incubated at 4° C. for 1 h. B cells (721.221) were washed with PBS and counted, then resuspended at a cell density of 1.3×10⁶/mL (50×10³ cells/well) in Fc Block (10% normal goat serum (Sigma, No. G6767), 2.5% FBS, 1% mouse anti-human CD32 (BD Biosciences, No. 555447), 2 mM EDTA, 0.05% NaN₃ in PBS) in FACS Buffer, then incubated at RT for 30 min. The cells were then washed with FACS Buffer (100 μL/well) and the plates were centrifuged at 450×g for 5 min to pellet. Supernatant was removed and a second wash with FACS Buffer at 250 μL per well was used, then centrifuged again at 450×g for 5 min to pellet.

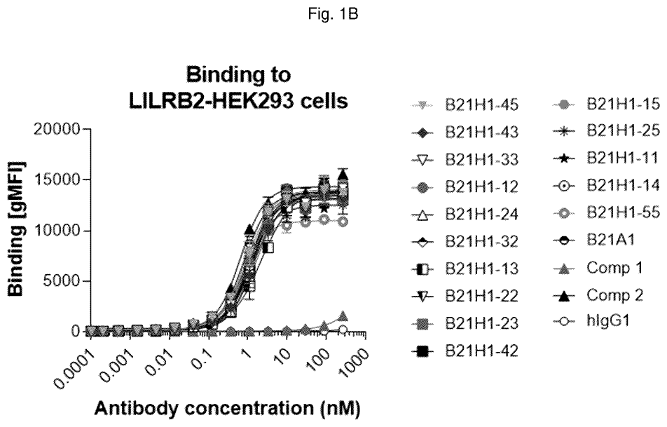

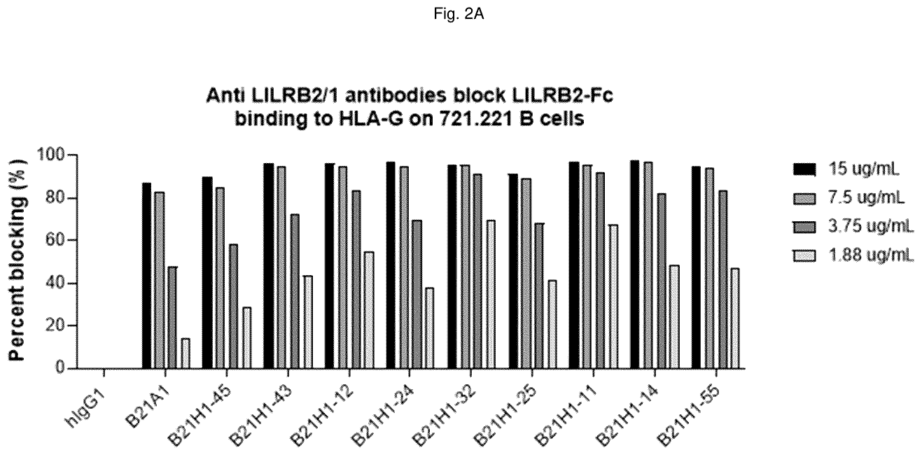

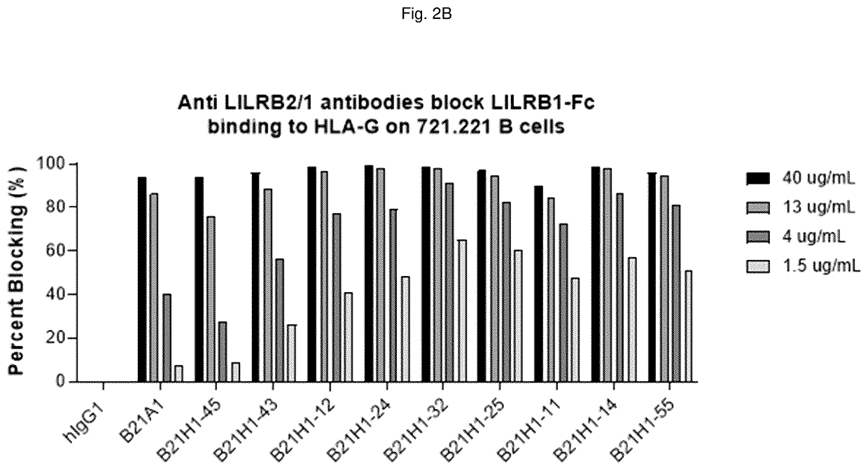

For all blocking assays, Streptavidin-PE (BioLegend, No. 405204) at a 1:250 dilution (75 μL/well) was added, and the cell/antibody mixture was incubated for 30 min at RT, protected from light. Cells were then washed twice in PBS containing 1 mM EDTA at 200 μL/well volume, then centrifuged at 450×g for 5 min to pellet cells. Cells were resuspended in 50 μL cell Zombie Violet™ viability dye at a 1:2000 dilution, and incubated for 10 min at RT, protected from light. A final wash of 200 μL/well FACS Buffer was performed, then cells were pelleted by centrifugation at 450×g for 5 min. Supernatant was removed and cells were resuspended in 100 μL FACS Buffer for flow cytometry analysis, as described in Example 6. The dual anti LILRB2/1 clones blocked the binding of LILRB1-Fc and LILRB2-Fc to HLA-G-expressing 721.221 cells in a dose-dependent manner, whereas the IgG1 isotype control did not affect LILRB2-Fc (FIG. 2A) or LILRB1-Fc (FIG. 2B) binding to 721.221 cells.

### Example 9: Isolation and Differentiation of Human Primary Cells

The evaluation of anti-LILRB2/1 antibodies in immunological assays requires the isolation of human T cells and monocytes, as well as the differentiation of monocytes into immune suppressive macrophages. Various techniques are known in the art, such as that described below for cells used in these examples. Apheresis products are collected from subjects and autologous monocytes and T cells are isolated using the technique described or another technique commonly used in the art. Briefly, human monocytes and T cells are isolated from white blood cells (WBCs) according to standard techniques. (LeukoPak, No. 4510-01 Full LeukoPak, BloodWorks Northwest, Seattle, WA). Peripheral blood mononuclear cells (PBMCs) are purified from LeukoPaks by standard density gradient centrifugation (FicollPaque® Premium 1.073, or 1.077, GE Healthcare, No. 17-5449-52, or Cytiva No. 17144003). The supernatant is discarded, and the pellet resuspended in 20 mL EasySep™ Buffer (STEMCELL Technologies, No. 20144) for counting of PBMCs and for further isolation of monocytes and T cells. Monocytes are isolated using the EasySep™ Human Monocyte Isolation kit (STEMCELL Technologies, No. 19359) following the manufacturer's instructions. Total CD8⁺ T cells are isolated using the EasySep™ Human CD8⁺ T Cell Isolation kit (STEMCELL Technologies, No. 17953), and total NK cells are isolated using the EasySep™ Human NK Cell Isolation kit (STEMCELL Technologies, No. 17955), following the manufacturer's instructions. These negative selection kits use antibodies to label undesired cell types for removal, allowing the desired target cells to be isolated from the sample untouched.

### Example 10: Macrophage Generation

Macrophages can be generated from PBMC-derived monocytes with commonly used techniques, such as that described below.

Generation of M0 Macrophages: At day 0, monocytes from individual subjects (isolated as described in Example 9) were plated in 96-well culture plates (Thermo Fisher (Costar), No. 09-761-175), at 25-50×10³ cells/100 μL/well in M0 Culture Medium (90% X-VIVO™ 15+10% FBS+100 ng/mL human M-CSF (PeproTech, No. 300-25)). Cells were incubated at 37° C. and 5% CO₂ for 5 to 6 days to produce M0 macrophages.

Generation of immune suppressive M2c macrophages: At Day 5 of culture, M0 macrophages were polarized to M2c macrophages by aspirating the medium gently from each plate and replacing it with 100 μL/well of M2c Culture Medium (M0 Culture Medium+20 ng/mL human IL-10 (PeproTech, No. 200-10)). Cells were incubated at 37° C. and 5% CO₂ for 2 days. At Day 7-8 of culture, the M2c macrophages were ready for coculture assay setup. M2c macrophages were detached from the plate by incubation with Macrophage Detachment Solution DXF (PromoCell, No. C-41330) and washed in PBS prior to downstream assays.

### Example 11: Generation of Exhausted T Cells

Exhausted T cells are an indicator of an immune-suppressive tumor microenvironment and contribute to cancer immune evasion. To mimic exhausted T cells in the TME, exhausted T cells having a blast-like morphology were generated from human PBMCs by repeated (3×) phytohemagglutinin (PHA) stimulation. Cells were counted and incubated at 1×10⁶ cells/mL in T cell blast culture medium (90% IMDM (Thermo Fisher (Gibco), No. 12440053)+10% human serum+2 μg/mL PHA-L (Sigma-Aldrich (Roche), No. 11249738001)+4 ng/mL recombinant human IL-2, (R&D Systems, No. 202-IL)). Cells were split 1:2 or 1:3 every 3 to 4 days and cultured for 10 days total (2 splits over 10 days; 3 total PHA stimulations). Fresh T cell blast culture medium was added to the cells at each split. The cells were harvested on day 10 and either set up for coculture assays or frozen for future use. Exhausted T cell phenotype was confirmed by expression of PD-1, TIM-3, and TIGIT, as well as the transcription factor EOMES (data not shown).

### Example 12: LILRB2/1 Antibodies Bind to Monocytes, M0 and M2c Macrophaqes

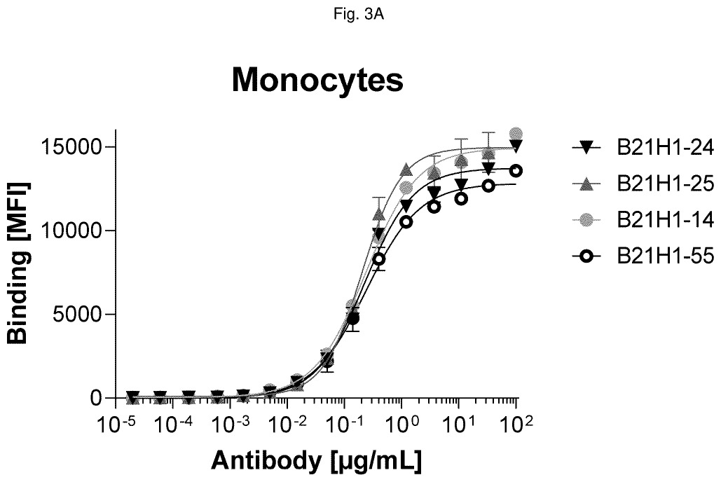

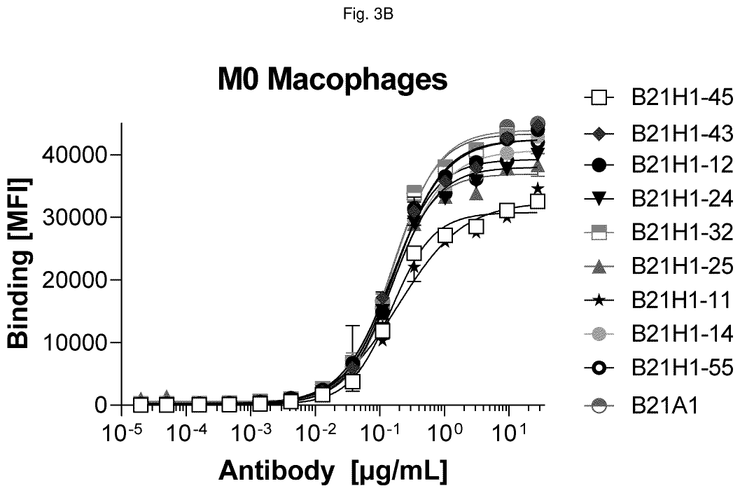

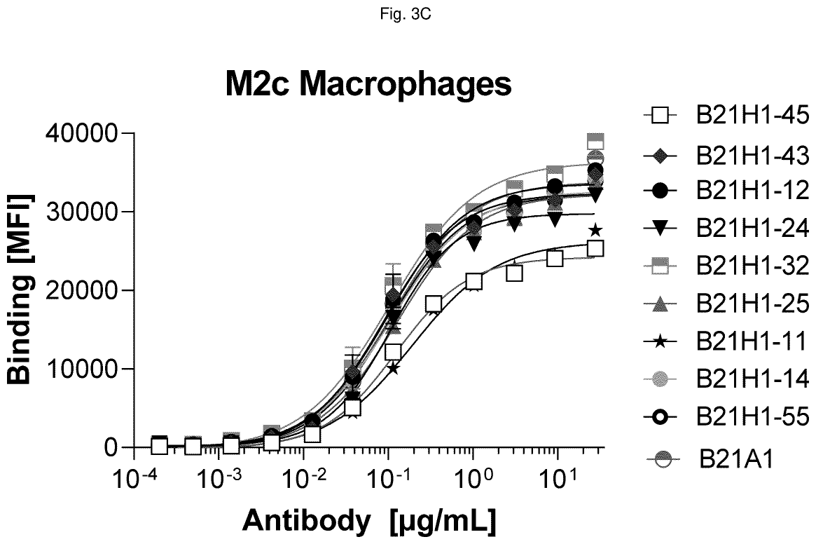

Anti-LILRB2/1 antibodies were evaluated for their ability to bind myeloid cell subsets expressing LILRB1 and LILRB2, including monocytes, M0 and M2c macrophages. M2c macrophages are used as a surrogate for tumor-associated macrophages, a suppressive macrophage commonly found in the tumor microenvironment. Frozen monocytes were removed from storage in vapor phase liquid nitrogen and thawed by gentle swirling in a water bath at 37° C., then resuspended in Assay Medium (X-VIVO™ 15 medium+10% FBS). PBMCs were placed in a 15 mL conical tube and centrifuged at 300×g for 5 min, then supernatant was removed. Cells were resuspended in Assay Medium at a cell density of 2.5×10¹ cells/mL. Cells were plated into 96-well flat bottom plates at 100 μL/well (25K cells/well). M0 and M2c macrophages were generated as described in Example 11 and collected from flasks by incubation for 15 min at RT in Macrophage Detachment Solution DXF, then removed from the flask into Assay Medium. Following centrifugation, the cells were resuspended in Blocking Buffer B (FACS Buffer+10% FBS+0.5 mg/mL human IgG1+0.05% NaN₃, 5% (w/v) then incubated at 4° C. for 30 min. Cells in 25 μL Blocking Buffer B were transferred to a 384-well plate at 2.5×10⁴ cells/well and 25 μL of titrated AF647-conjugated LILRB2 antibodies or AF647-conjugated IgG1 isotype control were added directly to each well at 2× final assay concentration. Cells were incubated with antibodies at 4° C. for 1 h. Cells were washed two times with FACS Buffer, stained with Zombie UV™ viability dye (BioLegend, No. 423107) (1:500 dilution) for 15 min at RT in the dark, washed with FACS Buffer and resuspended in 200 μL FACS Buffer for acquisition by flow cytometry as described in Example 6. GraphPad Prism was used for EC₅₀ binding calculations, showing an average EC₅₀ for two subjects tested (Table 9). B21A1 and humanized LILRB2/1 variants bind to human monocytes (FIG. 3A), M0 macrophages (FIG. 3B), and M2c macrophages (FIG. 3C) in a dose-dependent manner. A representative subject for each cell type is shown in dose titration graphs in FIG. 3.

TABLE 9
EC₅₀ values of LILRB2/1 antibody (IgG1) binding
to monocytes, M0 and M2c macrophages
Cell binding EC₅₀ [ng/mL]

| mAb | Monocytes | M0 macrophages | M2c macrophages |
|---|---|---|---|
| B21A1 | | 307 | 117 |
| B21H1-45 | | 196 | 115 |
| B21H1-43 | | 188 | 90.1 |
| B21H1-33 | | 235 | 132 |
| B21H1-12 | | N/A | 122 |
| B21H1-24 | 228 | 139 | 110 |
| B21H1-32 | | 340 | 124 |
| B21H1-13 | | 190 | 107 |
| B21H1-22 | | 163 | 91.2 |
| B21H1-23 | | 175 | 114 |
| B21H1-42 | | 130 | 83.4 |
| B21H1-15 | | 121 | 120 |
| B21H1-25 | 212 | 152 | 124 |
| B21H1-11 | | 2580 | 204 |
| B21H1-14 | 199 | 163 | 113 |
| B21H1-55 | 207 | 121 | 119 |
| Comp 1 | 823 | 93.7 | 86.2 |

### Example 13: LILRB2/1 Antibodies Bind to Immune Cell Subsets in Human PBMC

To further characterize the binding of LILRB2/1 antibodies to LILRB1 expressing immune cell subsets (namely, B, T, and NK cells), binding assays were performed with primary human PBMCs. Single cell suspensions of peripheral blood mononuclear cells (PBMCs) from healthy donors were seeded at 0.5 million per well in 96-well plates (Thermo Fisher, No. 249946). PBMCs were blocked with 0.5 mg/mL of human IgG1 myeloma plasma (Athens Research, No. 16-16-090707-1M) in FACS Buffer (PBS containing 2% FBS, 2 mM EDTA and 0.05% NaN₃) for 45 min to 1 h at 4° C. The immune cell phenotyping antibodies and indicated AlexaFluor647-labeled test antibodies were then added to the cells and incubated for 45 min to 1 h at 4° C. Antibodies used for immune cell phenotyping included: BUV496 anti-human CD14 (BD Biosciences, No. 741200), BV711 anti-human CD4 (BioLegend, No. 300558), BV786 anti-human CD16 (BD Biosciences, No. 563690), FITC anti-human CD56 (BioLegend, No. 318304), PE anti-human CD8 (BioLegend, No. 301008), PE-Cy7 anti-human CD19 (BioLegend, No. 302216), APC/Fire750 anti-human CD3 (BioLegend, No. 300470).

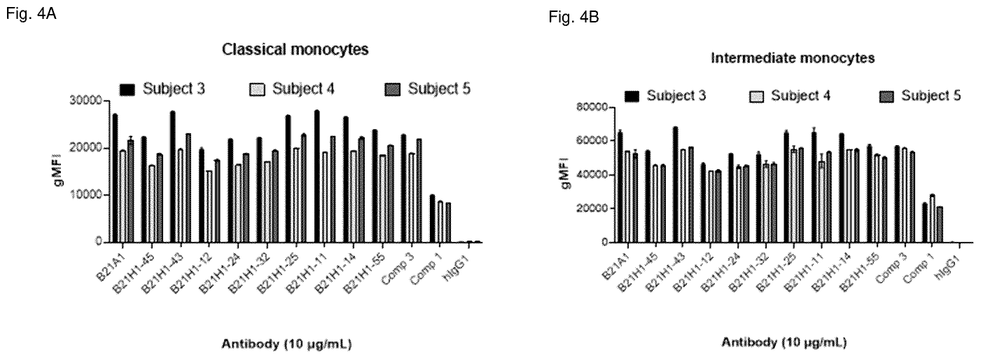

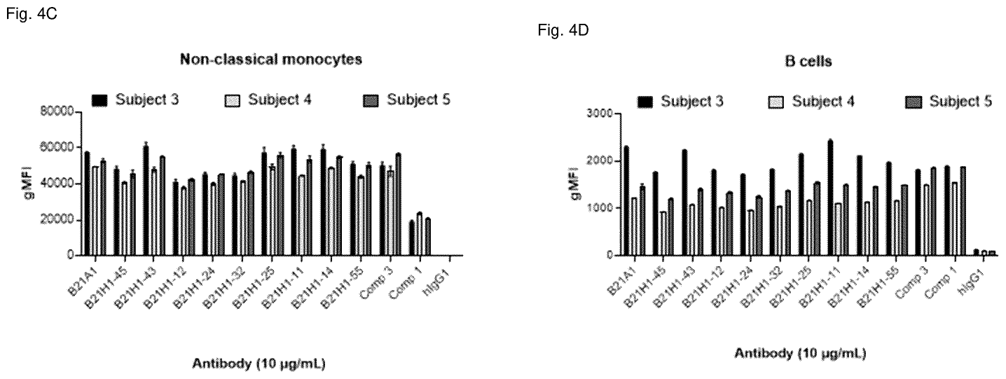

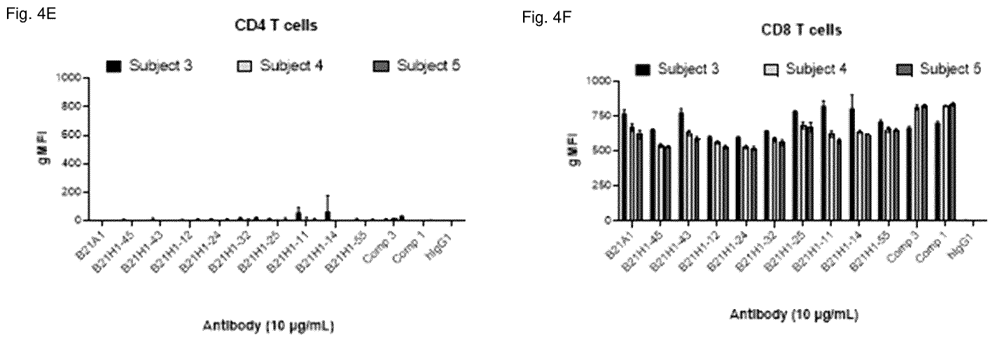

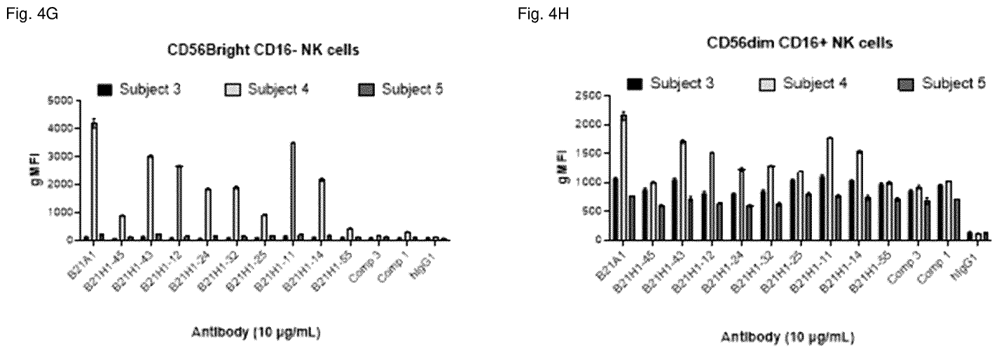

Cells were washed 1× with PBS and stained with the Zombie Violet™ fixable viability dye for 15 min at RT for live cell discrimination. Cells were washed 1× with FACS Buffer, fixed, and acquired on a FACS Symphony cytometer (BD Biosciences). Data analysis was performed using the FlowJo software (v10.8.1). Gating was performed on the live cell population to identify classical monocytes (Side Scatter (SSC)^{High}, CD3⁻, CD19⁻, CD14⁺, CD16⁻), intermediate monocytes (SSC^{High}, CD3⁻, CD19⁻, CD14⁺, CD16⁺), nonclassical monocytes (SSC^{High}, CD3⁻, CD19⁻, CD14⁻, CD16⁺), B cells (SSC^{Low}, CD19⁺), CD4 T cells (SSC^{Low}, CD19⁻, CD3⁺, CD4⁺, CD8⁻), CD8 T cells (SSC^{Low}, CD19⁻, CD3⁺, CD4⁻, CD8⁺), CD56^{Bright} NK cells (SSC^{Low}, CD19⁻, CD3⁻, CD56^{Bright}, CD16⁻), and CD56^{Dim} CD16⁺ NK cells (SSC^{Low}, CD19⁻, CD3⁻, CD56^{Dim}, CD16⁺). LILRB2/1 antibodies were observed to have bound classical, intermediate, and non-classical monocytes, as well as B cells, CD8⁺ T cells, and CD56^{dim} CD16⁺ NK cells (FIG. 4A-D, 4F, 4H). Very little binding was observed on CD4⁺ T cells (FIG. 4E), and variable binding, dependent on subject, was observed on CD56^{bright} CD16⁻ NK cells (FIG. 4G).

### Example 14: LILRB2/1 Antibodies Enhanced IFN-γ Response in LPS-Stimulated PBMC

To determine if LILRB2/1 blockade boosts the innate immune response, which in turn enhances IFN-γ secretion and reduces IL-10 release to abate the immune suppressive TME, we evaluated the effect of LILRB2/1 antibodies on the IFN-γ secretion and IL-10 secretion by PBMC in response to lipopolysaccharide (LPS).

Frozen PBMC were removed from storage in vapor phase liquid nitrogen and thawed by gentle swirling in a water bath at 37° C., then resuspended in Assay Medium. PBMC were placed in a 15 mL conical tube and centrifuged at 300×g for 5 min, then supernatant was removed. Cells were resuspended in Assay Medium at 3×10⁶ cells/mL. Cells were plated into 96-well flat bottom plates at 100 μL/well (300K cells/well). This cell density is optimized for the average level of IFN-γ response per subject. Fewer cells were plated per well if the subject was known to yield a higher cytokine response. Cells were rested at 37° C., 5% CO₂ for 1 h.

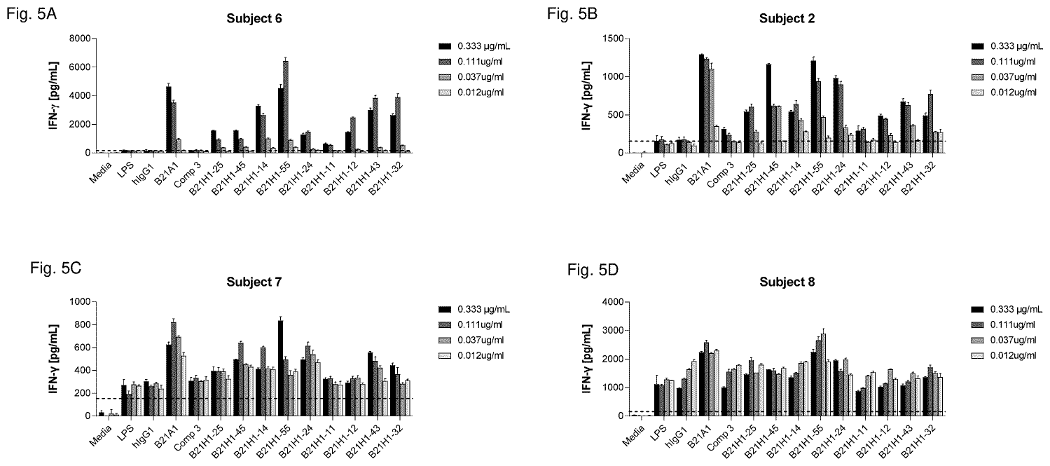

The LILRB2/1 antibodies were diluted to a 4× concentration (final concentration of 1 μg/mL) in X-VIVO™ 15 and 50 μL/well diluted antibody was added. For wells that did not receive antibody, 50 μL/well of X-VIVO™ 15 was added. The plates were incubated at 37° C., 5% CO₂ for 2 h prior to LPS stimulation. LPS (InvivoGen, No. Tlrl-pb5lps) was diluted in X-VIVO™ 15 to a 4× concentration (final concentration of 1 μg/mL in assay), and 50 μL/well was added to the cell/antibody mixtures, except that for subject samples that had a known higher IFN-γ response, LPS final concentration was 0.1 μg/mL. For control wells (no LPS), 50 μL/well of X-VIVO™ 15 was added. Cells were incubated at 37° C., 5% CO₂ for 24 h, at a final volume of 200 μL/well. Supernatant (150 μL) was harvested following 24-hour incubation and either frozen at −80° C. or directly tested for IFN-γ secretion and IL-10 secretion by ELISA (R&D systems). LILRB2/1 antibodies induced a pro-inflammatory innate Th1-like phenotype, in which the PBMC displayed enhanced IFN-γ secretion and reduced IL-10 release when stimulated with LPS. All LILRB2/1 antibodies evaluated showed enhanced IFN-γ secretion compared to isotype control, with variability observed between donors. Data from four representative subjects for enhancement of IFN-γ secretion are shown in FIGS. 5A-5D. Data from three representative subjects for reduced IL-10 release are shown in FIG. 14A-14C.

Combinations of LILRB2/1 antibodies with a Toll-like receptor 2 (TLR) ligand such as heat-killed Listeria monocytogenes (HKLM, InvivoGen, No. Tlrl-hklm, 2.5×10⁷ cells/mL) and Pam3CSK4 (InvivoGen, No. Tlrl-pms, 100 ng/mL) were also tested. The LILRB2/1 antibodies enhanced IFN-γ secretion and reduced IL-10 release by HKLM (2.5×10⁷ cells/mL) or Pam3CSK4 (100 ng/mL) treated PBMCs (data not shown).

### Example 15: LILRB2/1 Antibodies Enhanced TNF-α Secretion in CD40-Activated Macrophaqes

CD40L expressed on T cells is the primary molecule responsible for the activation of macrophages in the TME, through binding to CD40 expressed on macrophages via cell-to-cell contact. CD40/CD40L interactions are important for activating macrophages to behave as effector cells that mediate inflammation in T cell-mediated inflammatory processes. We developed an assay to assess inflammatory cytokine production by macrophages treated with LILRB2/1 antibodies, but without the presence of T cells.

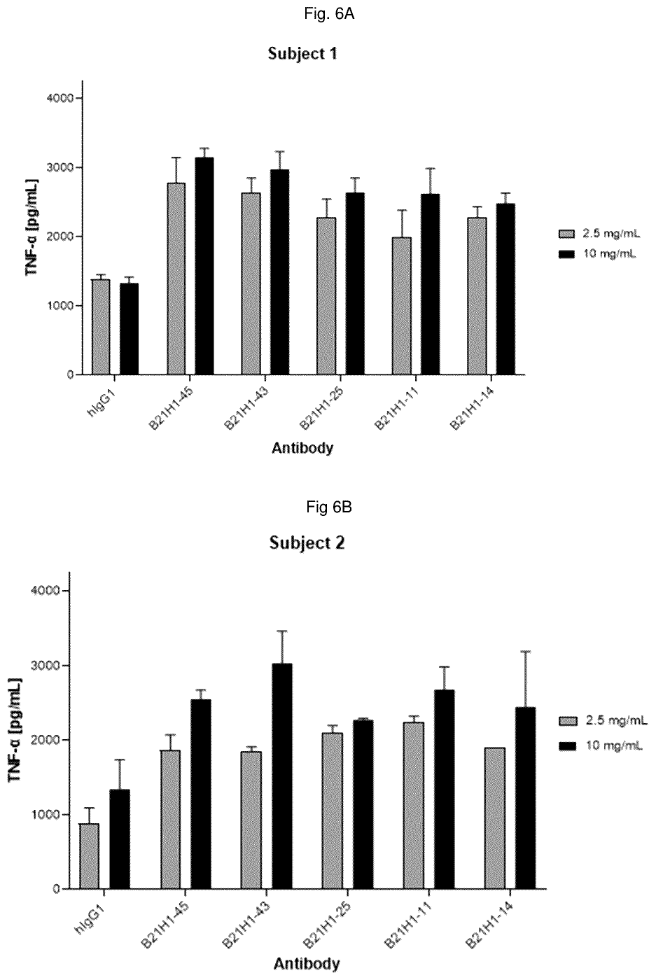

A HEK-293 cell line modified to express CD40L (CrownBio, No. C2041) was used to mimic the binding and subsequent stimulation by an activated T cell. (Alternatively, a stable HEK-293 cell line expressing CD40L can be generated using conventional means.) M0 macrophages were generated from monocytes (25K/well) as described in Example 11. Medium was removed from the wells and 100 μL/well fresh Assay Medium was added. LILRB2/1 antibodies at a 5× concentration (final concentration of 5 and 10 μg/mL) were added at 50 μL/well, and incubated onto macrophages at 37° C., 5% CO₂ for 2 h. During the 2-hour incubation, CD40L-expressing HEK-293 cells were harvested from flasks, and irradiated at 40 grey. Following pre-incubation with LILRB2/1 antibodies, CD40L-expressing HEK-293 cells (5×10¹) were added at a volume of 100 μL/well, bringing the final well volume to 250 μL/well in 96-well plates, and incubated overnight at 37° C., 5% CO₂. Following incubation, 200 μL of supernatant was harvested from wells and either frozen at −80° C. or immediately assayed for TNF-α secretion by Homogenous Time Resolved Fluorescence (HTRF) assay, according to manufacturer instructions (CisBio, No. 62HTNFAPET). All LILRB2/1 antibodies tested show comparable potentiation of TNF-α secretion by CD40L-activated macrophages, shown by two representative subjects, at both 5 and 10 μg/mL (FIG. 6A-6B).

### Example 16: LILRB2/1 Antibodies Relieve M2c-Mediated Immune Suppression in M2c/CD8⁺ T Cell Coculture Assay

The crosstalk between LILRB1- and LILRB2-expressing immunosuppressive myeloid cells and T cells contributes to T cell exhaustion and the lack of anti-tumor immune response in the TME. This crosstalk can be modeled by in vitro co-culture of autologous monocyte-derived M2c macrophages and anti-CD3-stimulated CD8⁺ T cells. Reduction of immune suppression can be evaluated by quantitation of T cell proliferation, and measurement of IFN-γ secretion as a surrogate of T cell activation and anti-tumor activity. After polarization of M0 macrophages to M2c macrophages as described in Example 11, supernatants were removed from the macrophages in 96-well culture plates and replaced with 100 μL Assay Medium containing a final concentration of 0.25 μg/mL OKT3 (BioLegend, No. 317326. LILRB2/1 antibodies (a dose titration from 40-0.63 μg/mL) were added at a volume of 50 μL/well, then plates were incubated at 37° C., 5% CO₂ for 1-2 h. While the M2c macrophages were incubated with the antibodies, autologous CD8⁺ T cells were isolated from PBMCs as described in Example 9. Isolated CD8⁺ T cells were labeled with 1:2000 CellTrace™ violet (Thermo Fisher, No. C34557) diluted in 1×PBS, then incubated at 37° C. for 20 min. Following CellTrace™ incubation, pre-warmed medium is added at five times the volume of labeled cells to quench CellTrace™ dye. Excess CellTrace™ was washed off with a 5× volume of pre-warmed Assay Medium and labeled CD8⁺ T cells were centrifuged at 300×g, then resuspended in Assay Medium, then added to the M2c/antibodies preparation at 5×10⁵ cells/mL in volume of 100 μL at 1:1 ratio of M2c:CD8⁺ T cells. Cells were then incubated at 37° C., 5% CO₂ for 72 h.

Supernatants containing T cells were transferred to a V-bottom 96-well plate, centrifuged to pellet the T cells, and culture supernatants were collected and frozen at −80° C. for quantification of human IFN-γ secretion by MSD-ELISA (MSD, U plex assay). T cell pellets were stained with e780 viability dye for 10 min at RT in the dark, washed with 150 μL FACS Buffer, and resuspended in 100 μL of FACS Buffer for acquisition on BD FACSymphony™ or FACSCanto™ flow cytometer (BD Biosciences). The percentage and overall count of proliferating CD8⁺ T cells was analyzed using FlowJO software and reported as total count of CellTrace⁺ dividing cells.

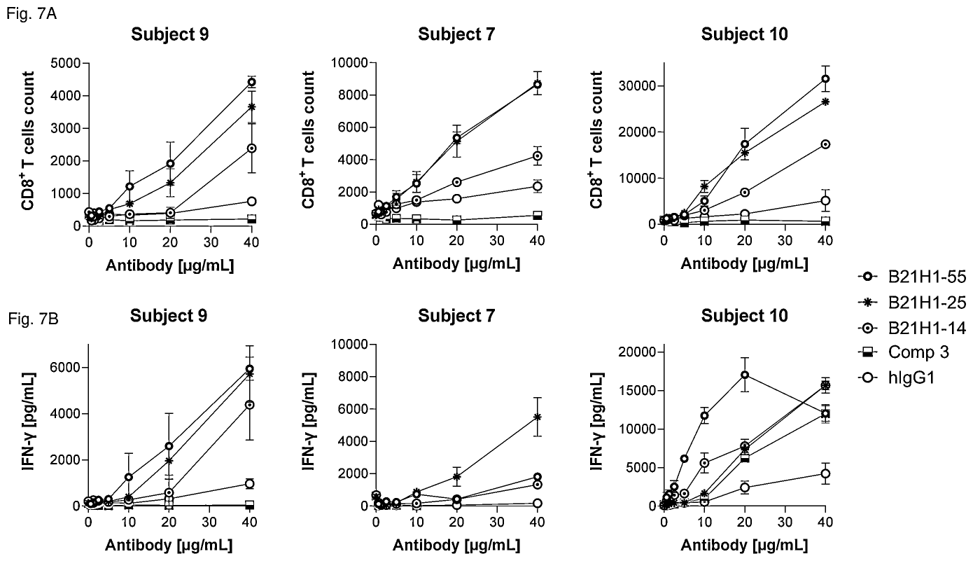

LILRB2/1 antibodies relieved M2c-macrophage-mediated immune suppression in M2c/T cell coculture assays, as measured by restoring CD8⁺ T cell proliferation and IFN-γ secretion (FIGS. 7A and 7B, respectively), for three representative subjects. For comparison, a LILRB2/1 antibody (Comp 3; VH SEQ ID NO: 47 and VL SEQ ID NO: 48) failed to restore T cell proliferation and enhance IFN-γ secretion (FIGS. 7A and 7B, respectively).

### Example 17: LILRB2/1 Antibodies Rescue IFN-γ Responses of Exhausted T Cells in Coculture with M2c Macrophaqes

A hallmark of an ineffective anti-cancer immune response is T cell exhaustion in the tumor microenvironment. Exhausted T cells are T cells with decreased cytokine expression and effector function. Reversing T-cell exhaustion and restoring anti-tumor potential represents a promising strategy to treat cancer. We evaluated the ability of LILRB2/1 antibodies to rescue functional activity of exhausted T cells from LILRB2/1-mediated immune suppression by an assay that utilizes exhausted T cells and M2c cells in a coculture to measure the immune suppression mediated by the macrophages.

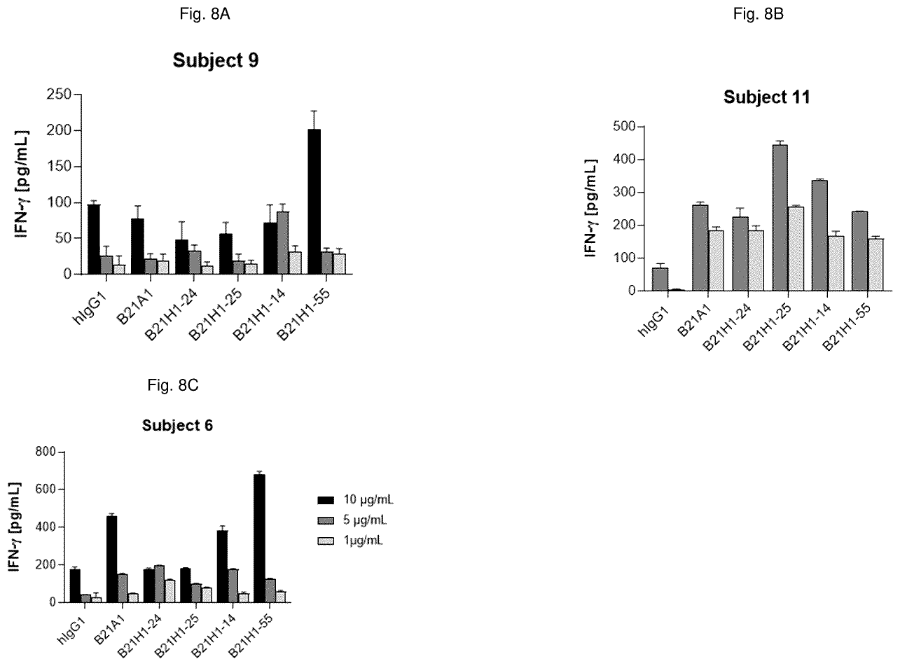

A coculture assay with exhausted T cells and M2c macrophages was used to measure the ability of the LILRB2/1 antibodies to rescue functional activity of T cell blasts from M2c-mediated immune suppression. After polarization of 50,000 cells/well M0 macrophages to M2c macrophages as described in Example 11, supernatants were removed from the macrophages in 96-well culture plates and replaced with Assay Medium containing LILRB2/1 antibodies or isotype controls, and the cells incubated at 37° C., 5% CO₂ for 2 h. OKT3 antibody, at a final concentration of 0.25 μg/mL, was added to wells and incubated at 37° C., 5% CO₂ for 30 min. T cell blasts (Example 12) were added last to M2c/LILRB2/1 antibody mixture+OKT3 at a 1:1 ratio and incubated at 37° C., 5% CO₂ for 72 h. IFN-γ levels were quantitated from supernatants collected 72 h after OKT3 stimulation by ELISA. B21A1 and humanized IgG1 variants rescued the IFN-γ response of exhausted T cells from M2c macrophage-mediated immune suppression in a dose-dependent manner, as shown for three representative subjects (FIG. 8).

### Example 18: B21A1 and Humanized Variants Enhance NK Cell Cytotoxicity

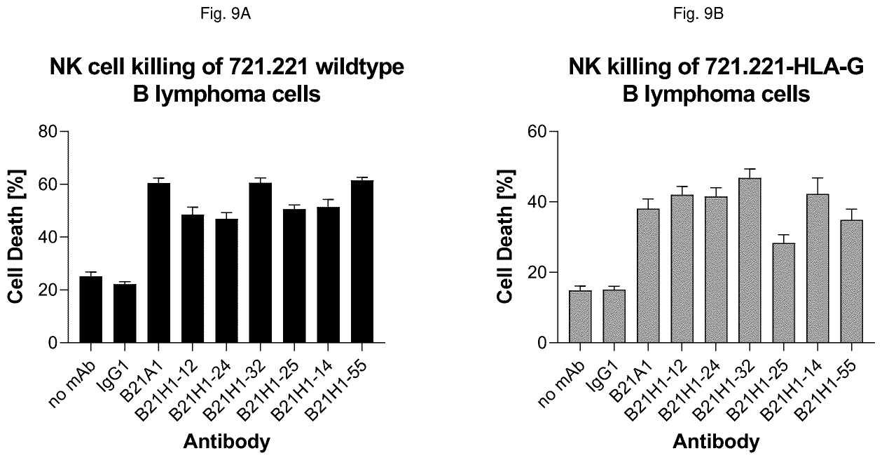

The ability of LILRB2/1 antibodies to enhance the cytotoxic capacity of NK cells was tested in primary human NK cells from healthy donors. Primary NK cells were isolated from frozen PBMC using immunomagnetic negative selection, as described in Example 9. Isolated NK cells were resuspended in 5 mL X-VIVO™ 15 medium+10% FBS+100 IU/mL (50 ng/mL) IL-2 (Peprotech, No. 200-02), then transferred to a T25 flask and incubated at 37° C., 5% CO₂ overnight. Target cancer cells (721.221 wild-type or 721.221 expressing HLA-G) were harvested from flasks and resuspended in Assay Medium, then counted, using a 1:1 dilution with Trypan blue (Thermo Fisher, No. 15250061). 721.221 WT or 721.221-HLA-G cells were labeled with CellTrace™ violet (Thermo Fisher, No. C34557) as described in Example 17. Excess CellTrace™ was washed off with pre-warmed Assay Medium and labeled target tumor cells were resuspended in Assay Medium. NK cells were plated into a 96-well plate in a volume of 50 μL/well at a final cell density of 50,000 cells/well. LILRB2/1 antibodies were plated into wells with NK cells at a 2× concentration in 50 μL/well, and pre-incubated for 1 h at 37° C., 5% CO₂. 721.221 cells were plated onto NK cells at a volume of 50 μL/well, for a final cell density of 10,000 target cells/well and a final ratio of 5:1 NK cells:target cells. The NK:target cell mixture was incubated for 4 h at 37° C., 5% CO₂. Following incubation, cells were transferred to V bottom 96 well plates, centrifuged at 300×g, and the supernatant was removed. Cells were resuspended in 1:2000 dilution of e780 viability dye in PBS (Thermo Fisher, No. 65-0865-18), and incubated at RT for 10 min in the dark. Following viability staining, FACS Buffer was added to wells at 150 μL/well, then centrifuged at 300×g for 5 min to remove dye. Supernatant was removed and 100 μL/well FACS Buffer was added/well to resuspend cells, for acquisition on BD FACSymphony™ or FACSCanto™ flow cytometer (BD Biosciences). The percentage of dead target cells was analyzed using FlowJO software and reported as percent cell death. An increase in NK cell-mediated target cell killing was observed against HLA-G-expressing (FIG. 9B) and wild-type (no HLA-G) (FIG. 9A) 721.221 cancer cells in the presence of LILRB2/1 antibodies, compared to isotype control.

### Example 19: LILRB2/1 Antibodies Elicit Minimal Cytokine Release in Whole Blood from Healthy Subjects

Immunomodulatory therapeutic antibodies carry the risk of cytokine release syndrome, a rapid systemic inflammatory response characterized by the secretion of inflammatory cytokines by immune cells. Antibody target binding may induce cytokines by directly activating lymphocytes and myeloid cells, or by interacting with Fcγ receptors on myeloid cells and NK cells. The whole blood in vitro cytokine release assay is a standard assay used to assess the risk of therapeutic antibody-mediated cytokine release syndrome. We evaluated whether treatment with humanized LILRB2 variants triggers the release of inflammatory cytokines in whole blood from healthy study subjects.

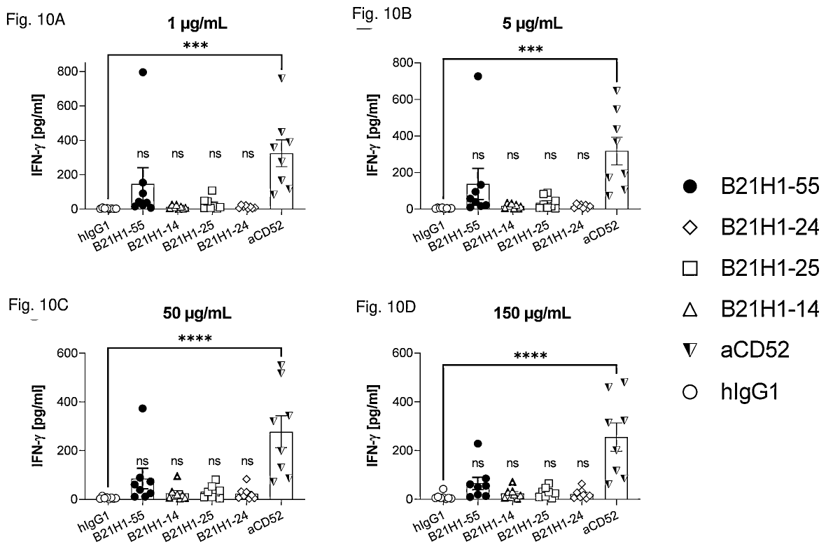

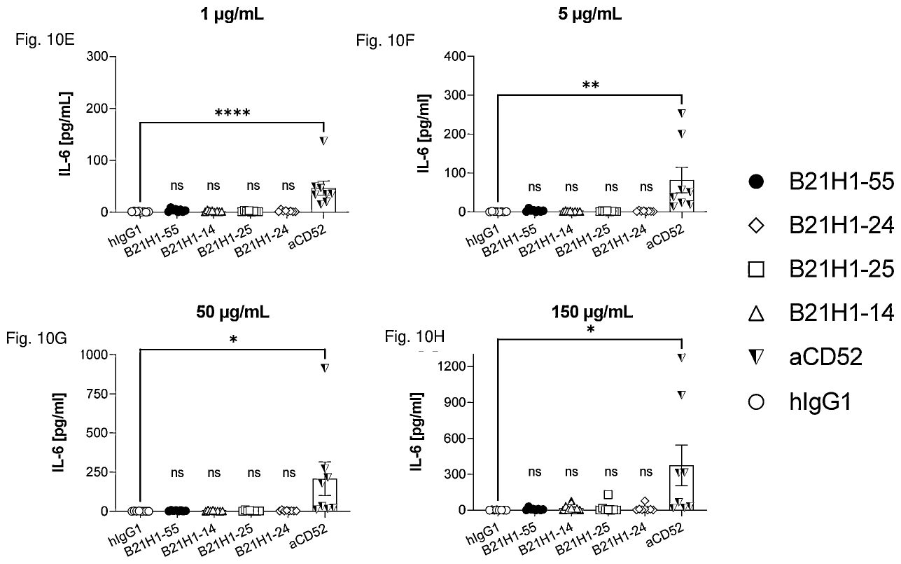

LILRB2/1 antibodies and control antibodies were diluted to a 10× final concentration in PBS in a dilution plate. Diluted LILRB2/1 antibodies (25 μL/well) were transferred to 96-well plates. Whole blood from healthy subjects was purchased (Bloodworks Northwest). Whole blood (225 μL/well) was added to LILRB2/1 antibodies, without mixing, to avoid lysing cells. Blood/antibody mixture was incubated at 37° C., 5% CO₂ for 24-48 h. Following incubation, plates were centrifuged at 350×g for 5 min to pellet cells. Plasma was harvested (65 μL) from the surface of each well, and immediately assessed for IL-6, TNF-α, IFN-γ, and IL-1β cytokine secretion by MSD-ELISA, according to the manufacturer's instructions (MSD). The release of IL-1β, IL-6, IFN-γ, and TNF-α in response to treatment of LILRB2/1 antibodies by whole blood cells from 8 subjects was compared to the cytokine induction by the corresponding human IgG1 isotype, no-treatment control, and anti-CD52 (Alemtuzumab; BOC Sciences, No. B0084-305393) positive control antibody. Treatment with the LILRB2/1 antibodies did not trigger the release of significant amounts of IFN-γ and IL-6 (FIG. 10A-10H) or TNF-α and IL-1β (data not shown) when compared to human IgG1 isotype control treatment. In contrast, the anti-CD52 antibody increased the release of IFN-γ and IL-6 significantly at all doses levels evaluated when compared to the IgG1 isotype control.

### Example 18: LILRB Antibodies Bind to Myeloid Cells, Granulocytes, and Lymohocytes in Whole Blood of Healthy Subjects

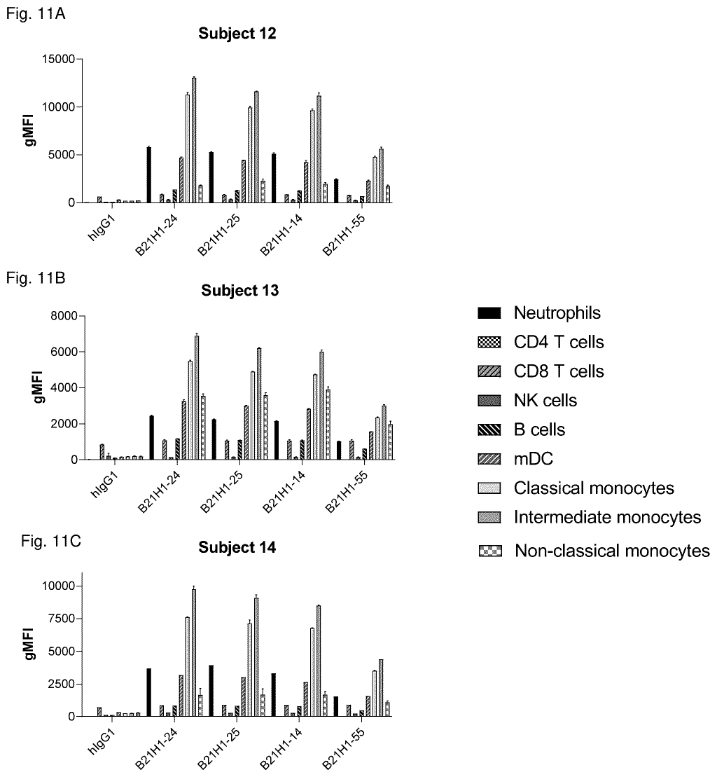

LILRB2 is exclusively expressed on whole blood myeloid cells such as monocytes and neutrophils, while LILRB1 is found on whole blood myeloid cells and lymphocytes. To determine that LILRB2/1 antibodies bind to relevant immune cell populations in whole blood, whole blood immunophenotyping assays were performed. Whole blood from healthy subjects was purchased (Bloodworks Northwest). Tubes containing blood containing heparin were gently inverted to evenly distribute plasma and cells, then blood was mixed with Blocking Mix (10% FBS+500 μg/mL Human IgG1 myeloma plasma (Athens Research)+0.05% NaN₃, 5% (w/v). Blood/Blocking Mix was then mixed thoroughly and pipetted into 96-well deep plates, then incubated for 1 h at 4° C. Cells were resuspended a second time at the 30-minute mark during incubation with Blocking Mix. Antibody titrations were made in FACS Buffer and added to blood at a final concentration of 1 and 0.1 μg/mL. Blood mixture was incubated with primary antibodies for 45 min at 4° C., protected from light. Following incubation, plates were brought to RT for 15 min, for an incubation of 1 h. Red blood cells were lysed by three consecutive RBC lysis steps with 1×RBC lysis buffer (BD Bio, No. 555899) by adding RBC lysis buffer to blood mixture, pipetting up and down to fully mix, then incubating for 10 min at RT, protected from light. Following incubation, cell plates were sealed, and centrifuged at 200×g for 5 min, then supernatant was aspirated from wells. 1×PBS was added to stop RBC lysis, then centrifuged at 200×g for 5 min, and supernatant removed. Cells were then transferred to 96 well V bottom plates. Cells were then resuspended in blocking buffer (10% FBS+1:10 dilution FcX block+FACS Buffer) and incubated at RT for 15 min. Flow cytometry fluorophore-conjugated antibody cocktail (BV421 anti-human CD3 (BioLegend, No. 300434), BV711 anti-human CD4, APC/Cy7 anti-human CD8 (BioLegend, No. 344714), PE anti-human CD11c (BioLegend, No. 337206), BUV496 anti-human CD14, BUV805 anti-human CD15 (BD Biosciences, No. 742057), BV786 anti-human CD16, BV605 anti-human CD19 (BioLegend, No. 302244), FITC anti-human CD56, and PE/Cy7 anti-human HLA-DR (BioLegend, No. 307616) (Table 10A) was added directly to wells on top of blocking buffer, then incubated at RT for 30 min, protected from light. Following incubation, FACS Buffer was added to wells for washing, then plates were centrifuged at 350×g for 5 min and resuspended in 350 μL FACS Buffer for acquisition on FACSymphony™ cytometer. Populations were identified according to gating parameters as described in Table 10B. Representative data from 3 subjects are shown in FIG. 11A-11C, in which LILRB2/1 antibodies were evaluated for binding at 1 μg/mL, to whole blood myeloid and lymphocytes cell populations. The LILRB2/1 antibodies bound to myeloid cells, including classical, non-classical, and intermediate monocytes, myeloid dendritic cells (mDCs), and neutrophils. B21A1 and humanized variants also bound human CD8⁺ T cells, B cells and NK cells (FIG. 11A-11C).

TABLE 10A
Antibody Panel I

| Marker | Antibody Clone | Fluorophore |
|---|---|---|
| CD3 | UCHT1 | BV421 |
| CD8 | SK1 | APC/Cy7 |
| CD4 | RPA-T4 | BV711 |
| HLA-DR | L243 | PE/Cy7 |
| CD19 | HIB19 | BV605 |
| CD56 | HCD56 | FITC |
| CD14 | MϕP9 | BUV496 |
| CD16 | 3G8 | BV786 |
| CD11c | Bu15 | PE |
| CD15 | W6D3 | BUV805 |

TABLE 10B
Immune Cell Lineages

| Cell Type | Gates |
|---|---|
| CD4⁺ T cells | CD15⁻ CD56⁻ CD3⁺ CD4⁺ |
| CD8⁺ T cells | CD15⁻ CD56⁻ CD3⁺ CD8⁺ |
| NK cells | CD15⁻ CD3⁻ CD56⁺ |
| B cells | CD15⁻ CD3⁻ CD56⁻ CD19⁺ HLA-DR⁺ |
| Neutrophils | CD15⁺ CD16⁺ CD14⁻ SSC^{high} |
| Classical | CD3⁻ CD56⁻ CD19⁻ CD15⁻ HLA-DR⁺ |
| monocytes | CD14⁺ CD16⁻ |
| Intermediate | CD3⁻ CD56⁻ CD19⁻ CD15⁻ HLA-DR⁺ |
| monocytes | CD14⁺ CD16⁺ |
| Non-classical | CD3⁻ CD56⁻ CD19⁻ CD15⁻ HLA-DR⁺ |
| monocytes | CD14⁻ CD16⁺ |
| Myeloid | CD3⁻ CD56⁻ CD19⁻ CD15⁻ CD14⁻ |
| dendritic cells | CD16⁻ CD11c⁺ HLA-DR⁺ |

### Example 21: Pharmacokinetic Profiling of LILRB2/1 Antibodies in Humanized FcRn Mice

Therapeutic monoclonal antibodies for cancer treatments are usually of the immunoglobulin G (IgG) subclasses. The half-lives of monoclonal antibodies tested in mice do not correlate with those observed in humans, because human IgG binds mouse neonatal Fc receptor (FcRn) with higher affinity than to human FcRn (hFcRn). FcRn is a major histocompatibility complex (MHC) class I-like heterodimer, containing an Fc binding domain and a 32 microglobulin (β2m). FcRn binds to the Fc portion of an IgG in the acidic environment of the lysosome to prevent its degradation, and FcRn-bound IgG antibodies are recycled back to the extracellular surface where, at physiological pH, the IgG dissociates from FcRn and goes back into circulation, which in turns prolongs half-life of these IgG monoclonal antibodies compared to IgG not bound to FcRn, which otherwise gets processed for degradation by lysosomes (Roopenian et al., Nat Rev Immunol. 2007 7:715-25; Roopenian et al., J Immunol. 2003 170:3528-33; Challa et al., Curr Top Microbiol Immunol. 2014 382:249-72). Pharmacokinetic studies of monoclonal antibodies in WT mice, FcRn humanize (hFcRn) mice and non-human primates (NHP) have demonstrated that the hFcRn mice were predictive of PK profile of antibodies in clinical setting than the WT mice (Avery L B et al., MAbs 2016).

Different FcRn transgenic mice have been created to knockout the murine FcRn and express human FcRn. Several groups have demonstrated that the PK of human therapeutic antibodies in these humanized FcRn mice correlates well with human PK and show comparable PK in non-human primates (Petkova et al., Int Immunol. 2006 18(12):1759-69; Tam et al., MAbs. 2013 5(3):397-405; Wang et al., Drug Metab Dispos 2011 39(9):1469-77; Roopenian et al., Methods Mol Bioll. 2010 602:93-104; Avery et al., MAbs 2016 8(6):1064-78; Proetzel et al., Methods. 2014 65(1):148-53). For example, the Tg32 and Tg276 mouse strains have been engineered on the C57BL/6 background. These mice are immunocompetent and have all murine immune cells; only the mouse FcRn is deleted and human FcRn is expressed in these models.

Male homozygous Tg32 FcRn mice were purchased at age 6-8 weeks (Jackson Laboratory, No. 014565) and housed in microisolator cages under specific pathogen-free conditions at the vivarium of Bloodworks Northwest. All procedures were carried out under the institutional guidelines of Bloodworks Northwest's Institutional Animal Care and Use Committee (IACUC) Protocol #5390-01. Mice were identified using ear tags. All mice were acclimated for a minimum of 5 days prior to start of antibody dosing. On the day of dosing, the initial weight was recorded for each mouse. Mice were assigned to three LILRB2 antibody treatment groups: B21A1 (IgG1 parental chimera), B21H1-25, and B21H1-55 with 3 mice/group for B21A1 and 5 mice per group for the B21H1-25 and B21H1-55 groups. Mice received a single intravenous (IV) bolus dose of 10 mg/kg of B21A1, B21H1-25, or B21H1-55 antibodies. Blood (20-50 μL) was collected from the lateral saphenous vein without application of anesthesia according to the approved IACUC protocol. The saphenous vein puncture method allows for repeat sampling from the same animal until reaching limit of no more than 1% of body weight per 2-week period. Blood was drawn from each mouse at the following time points: 0.15, 1, 2, 5, 24, 48, 100, 168, 240 and 336 h. Whole blood was allowed to clot at RT for a minimum of 30 min. Clots were removed by centrifugation (2000×g) for 10 min at 4° C. Serum was aliquoted into new microtubes and frozen at −80° C. until analysis.

Human LILRB2 capture ELISA (in accordance with the method of Example 3) was performed to determine antibody concentration for each group. Stocks of B21A1 and humanized variants (B21H1-25 and B21H1-55) were diluted in Assay Buffer (1% BSA in PBS) to prepare a 300 ng/mL top standard concentration. A 15-point, 2-fold serial dilution of standards were diluted in 0.01 to 0.2% mouse serum. Serum samples from anti-LILRB2/1 treated mice were also diluted in Assay Buffer. Different dilutions (1:1,000 to 1:40,000) were tested depending on time point of serum collection and dosing antibody. Each standard dilution and mouse serum dilution was assayed in duplicate wells.

Standard curves for B21A1, B21H1-25, and B21H1-55 were generated by plotting CPM vs antibody concentrations using Microsoft Excel and the add-in XLFIT (IDBS). Antibody concentrations for each sample time point (0.15 h to 336 h) at each dilution were extrapolated from the standard curve using XLFIT. The optimal sample dilution or dilutions were chosen such that antibodies are adequately detected by their corresponding standard curve. The final concentration of serum B21A1, B21H1-25, and B21H1-55 was adjusted to multiply by the sample dilution factor.

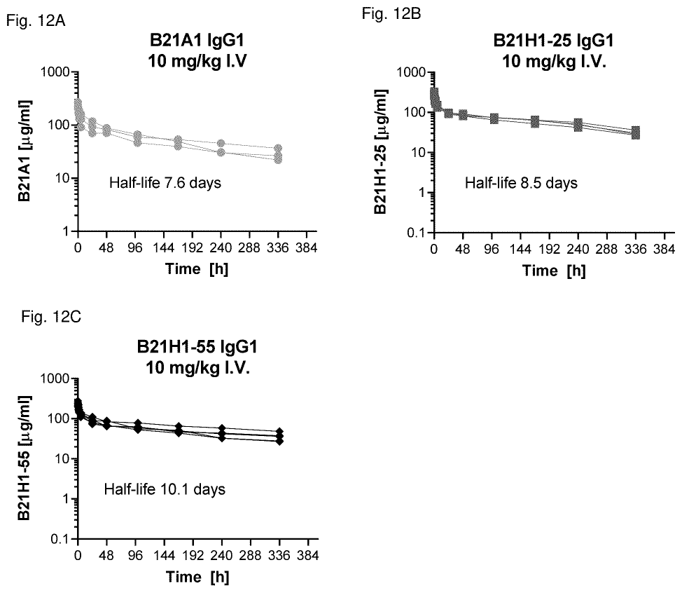

Mean serum concentrations (μg/mL) of B21A1 chimera IgG1 and the humanized variants B21H1-25 and B21H1-55 were calculated from 35 mice to generate a composite PK profiles (FIG. 12A-12C). Non-compartmental PK analysis after IV bolus injection was carried out with PK solver 2.0 add-in for Microsoft Excel® (Table 11). Antibody serum exposures were graphed with GraphPad Prism for Windows (GraphPad Software). The LILRB2/1 antibodies B21A1, B21H1-25, and B21H1-55 demonstrated half-lives of 7.6 days, 8.5 days and 10.1 days, respectively in the humanized FcRn mice.

TABLE 11
Parent and humanized IgG1 variants have typical
PK profiles in humanized FcRn mice

| Parameters | Unit | B21A1 | B21H1-25 | B21H1-55 |
|---|---|---|---|---|
| Dose | mg/kg | 10 | 10 | 10 |
| t_{1/2} | h | 183 | 204 | 242 |
| t_{1/2} | days | 7.6 | 8.5 | 10.1 |
| Tₘₐₓ | h | 0.15 | 0.15 | 0.15 |
| Cₘₐₓ | μg/mL | 235 | 285 | 236 |
| AUC₍₀₋₃₃₆₎ | μg/mL*h | 18218 | 21709 | 19285 |
| AUC_{(0-inf)} | μg/mL*h | 25740 | 30698 | 31662 |

### Example 22: LILRB2/1 Chimera Antibody Inhibits Tumor Growth in Humanized NSG-SGM3 Mice Bearing Subcutaneous Human SK-MEL-5 Melanoma

Anti-tumor efficacy of the B21A chimera IgG4 (B21A4) was tested in an in vivo humanized tumor model. Female humanized NSG-SGM3 mice were purchased from Jackson Laboratory (JAX West). The immune systems of the triple transgenic NSG-SGM3 mice expressing human IL-3, GM-CSF (CSF2) and SCF (KITLG) (Strain No. 013062) were reconstituted by intravenous injection of human UBC CD34⁺ hematopoietic stem cells into irradiated 3-week-old NSG-SGM3 mice. Engraftment of human CD45⁺ cells to assess humanization in peripheral blood was monitored weekly by Jackson Laboratory, and only animals with a minimum engraftment of 25% human CD45⁺ cells were received from Jackson Laboratory and enrolled in the study.

In vivo Tumor Xenograft Model and LILRB2/1 Antibody Dosing:

Female humanized NSG-SGM3 were housed in microisolator cages under specific pathogen-free conditions at the vivarium of Bloodworks Northwest. All procedures were carried out under the institutional guidelines of Bloodworks Northwest's IACUC Protocol #5390-02. Mice were identified using ear tags. All mice were acclimated for a minimum of 5 days prior to study initiation. Mice were inoculated subcutaneously (SC) into the right flank with 2×10⁶ SK-MEL-5 human melanoma cells (HLA class A*02:01; ATCC) in 100 μL PBS with 20% Matrigel (R&D Systems, No. 3632-005-02).

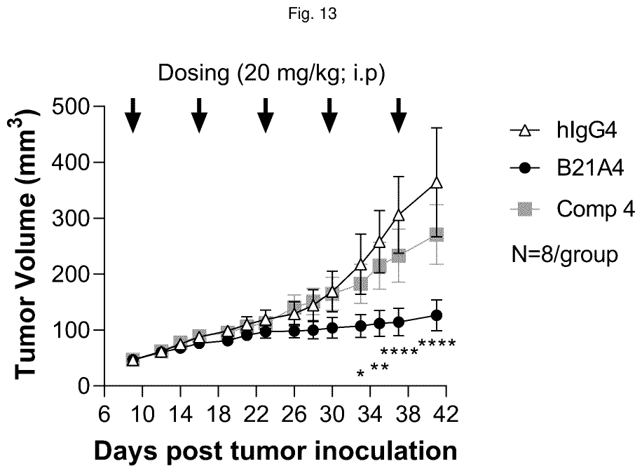

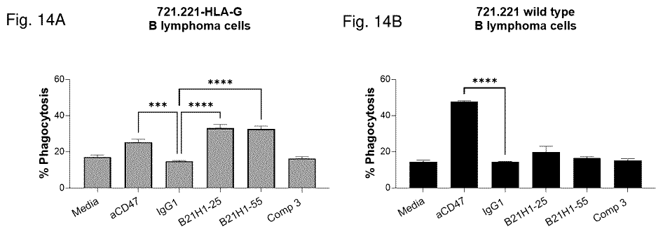

Tumor sizes were measured twice a week using a digital caliper. Tumor volumes were calculated: Tumor volume (mm³)=(L×W²/2) where L is the largest dimension and W is the smallest dimension. When an average tumor size of approximately 50 mm³ was reached, mice were randomized into groups based on both tumor size and engraftment of human CD45+⁺ cells, with each group containing 8-9 mice. Mice were dosed intraperitoneally (IP) with 20 mg/kg B21A4, a LILRB2 antibody (Comp 4; VH SEQ ID NO: 49 and VL SEQ ID NO: 50), or IgG4 isotype control on the day of randomization (Day 7) and again every 7 days for a total of 6 doses (days 9, 16, 23, 30, and 37 after tumor inoculation) (arrows in FIG. 13). Mice were sacrificed on day 41, for final tumor weight measurements.

Mean tumor growth inhibition (TGI), was calculated using the following equation:

$$TGI = [ 1 - \frac{( \overline{X} {Treated}_{( final )} - \overline{X} {Treated}_{( Day 0 )} )}{( \overline{X} {{Control}_{( final )}}_{{{-}^{}}^{}} \overline{X} {Control}_{( Day 0 )} )} ] \times 100 \%$$

Statistical differences in tumor volumes were confirmed using a parametric, RM (Repeated Measure) Two-way ANOVA with Geisser-Greenhouse correction using GraphPad Prism software. Standard errors of the mean were calculated for the tumor volumes daily. The P values were considered significant as follows: *P<0.05; **P<0.01 and ***P<0.001.

Mice treated with anti-LILRB2/1 B21A4 achieved 75% tumor growth inhibition (TGI) and 37.5% tumor regression by day 41 in comparison to IgG4 control-treated group, suggesting the ability of B21A4 to delay tumor growth (Table 12 and FIG. 13) and further suggesting that humanized variants of the antibody would have similar effect.

TABLE 12
In vivo tumor growth inhibition (TGI) and tumor
regression of B21A4 treatment in a SK-MEL-5
tumor model in humanized NSG-SGM3 mice

| | Tumor Growth Inhibition (%) | | | | | | Regression (%) |
|---|---|---|---|---|---|---|---|
| Group | d28 | d30 | d33 | d35 | d37 | d41 | d41 |
| B21A4 | 45 | 53 | 64 | 69 | 74 | 75 | 38% |
| Comp 4 | −5 | 3 | 16 | 17 | 24 | 26 | 11% |

### Example 23: Phagocytosis Assays

LILRB2 and LILRB1 bind to the classical (HLA-A and HLA-B) and non-classical (HLA-G, HLA-E and HLA-F) MHC class I molecules. The expression of the common MHC class I component 32-microglobulin (B2M) by cancer cells directly protect them from phagocytosis by macrophages. This protection is mediated by MHC class I binding to LILRB1 or LILRB2 by providing a “Don't eat me” signal, and disruption of MHC class I binding to LILRB1 or LILRB2 have been shown to potentiate phagocytosis of tumor cells (Barkal et al., Nat Immunol. 2018 January; 19(1):76-84. doi: 10.1038/s41590-017-0004-z. Epub 2017 Nov. 27. PMID: 29180808; PMCID: PMC5832354, Mandel et al., J Immunother Cancer. 2022 September; 10(9):e004859. doi: 10.1 136/jitc-2022-004859. PMID: 36096532; PMCID: PMC9472153, Tian et al., Cancer Immunol Res. 2024 Feb. 23. doi: 10.1158/2326-6066.CIR-23-0568. Epub ahead of print. PMID: 38393969).

The ability of LILRB2/1 antibodies to induce phagocytosis of tumor cells by macrophages was examined in vitro using 721.221 wild type B cell lymphoma tumor cells or 721.221 expressing HLA-G. Human monocytes were isolated from individual subjects (isolated as described in Example 9), plated in flat bottom 96-well plates in macrophage media (X-VIVO™ 15 supplemented with 10% FBS and 100 ng/mL M-CSF as described in Example 9) and incubated at 37° C., 5% CO₂ for 5 days until monocytes were differentiated to M0 macrophages, then polarized to M2c macrophages for 2 days at 37° C. (as described in Example 10). On day 7, the M2c macrophages were ready for phagocytosis assay set up. Medium from wells containing M2c was removed, then wells were rinsed 1× with PBS to remove non-adherent macrophages. LILRB2/1 antibodies or isotype control were added to macrophages at a final concentration of 20 μg/mL in 50 μL/well at 2× concentration (40 μg/mL) and incubated for 1 hr at 37° C. in 5% CO₂.

During the 1 hr incubation of macrophages with antibodies, the tumor target cells (721.221 wild type or 721.221-HLA-G-expressing), were labeled for 20 min at 37° C. with 1:2000 CellTrace™ violet (CTV; Thermo Fisher, No. C34557) diluted in 1×PBS (as described in Example 16). Following 20 min incubation, excess CellTrace™ was washed from target cells and cells were resuspend in media (X-VIVO™ 15+10% FBS), then added to M2c macrophages at 100,000 target tumor cells/well in 50 μL/well for a final 1:2 ratio of macrophages to tumors in a final 100 μL/well. Some wells received anti-CD47 phagocytosis positive control antibody (Magrolimab, Hu5F9, Selleckchem, No. A2036). Target cells, macrophages, and antibodies were incubated overnight for 18 hr at 37° C., 5% CO₂ to allow for phagocytosis.

The next day, media was discarded to remove non-phagocytosed cells; wells were rinsed 1× with PBS then supernatant discarded. Adherent macrophages were detached from wells by adding 50 μL/well 1×TrypLE™ Express Enzyme solution (Thermo Fisher, No. 12604013) and incubated at 37° C., 5% CO₂. Following 30 min incubation, cells were resuspended in 150 μL warm medium and transferred to V bottom 96 well plates, centrifuged at 300×g, and the supernatant was removed. Cells were resuspended in 1:2000 dilution of eFluor™ 780 (eF780) fixable viability dye in PBS (Thermo Fisher, No. 65-0865-18), and incubated at RT for 10 min in the dark. Following viability staining, FACS Buffer was added to wells at 150 μL/well, then centrifuged at 300×g for 5 min to remove dye. Cells were blocked in 25 μL/well Fc block (2.5 μL/well) in FACS buffer, incubated at 4° C. for 20 min, then PE-conjugated anti-CD14 (BioLegend, No. 325606) was added to each well at 2.5 μL/well, and incubated at 4° C. for 30 min. Following staining, FACS Buffer was added to wells at 150 μL/well, then centrifuged at 300×g for 5 min. Supernatant was removed and 100 μL/well FACS Buffer was added to resuspend cells, for acquisition on BD FACSymphony™ or FACSCanto™ flow cytometer (BD Biosciences). The percentage of phagocytosis of tumor cells was determined by reporting the percentage of live (eF780⁻) CTV⁺CD14⁺ cells using flow cytometry. An increase in M2c-mediated tumor phagocytosis was observed against the HLA-G-expressing tumor cells only in the presence of LILRB2/1 antibodies or anti-CD47 phagocytosis control antibody (FIG. 14A), compared to isotype control or media control. Phagocytosis in the presence of LILRB2/1 antibody was dependent on HLA-G as depicted by lack of tumor phagocytosis against wild type (no HLA-G) 721.221 cancer cells.

The ability of macrophages to induce phagocytosis of both the 721.221 wild type and 721.221-HLA-G⁺ target cells was confirmed with anti-CD47 positive control antibody (FIGS. 14A and 14B, in which *** is p=0.001 and *** is p=0.0001). Compared to isotype control, LILRB2/1 antibodies of the invention induced HLA-G-mediated phagocytosis of tumor cells by M2 macrophages (FIG. 14A) but not phagocytosis of the 721.221 wild type parental tumor cells (FIG. 14B). In contrast, a comparator LILRB2/1 antibody (Comp 3; VH SEQ ID NO: 47 and VL SEQ ID NO: 48) failed to induce macrophage phagocytosis of 721.221-HLA-G-expressing tumor cells or 721.221 parental cells.

All documents mentioned in this application are hereby incorporated herein by reference in their entirety.

SEQUENCES

| SEQ | | |
|---|---|---|
| ID NO | Name | Sequence |
| 1 | B21A-VH | QSVEESGGRLVTPGTPLTLTCTVSGFSLNTYAMTWVRQAPGKGLEWIGIIVASGGTYY |
| | | ASWAKGRFTISRTSTTVVMKITSPTTEDTATYFCARGGMGSDWGESLDAFDPWGQGTL |
| | | VTVSS |
| 2 | B21A-VL | DIVMTQTPASVEAAVGGTVTINCQASGNIYSFLAWYQQKPGQPPKLLIYTASNLETGV |
| | | PSRFSGSGSGTEYTLTISGVQCDDAATYYCLGGLYSSDVSYAFGGGTEVVVK |
| 3 | B21A-HC4 | QSVEESGGRLVTPGTPLTLTCTVSGFSLNTYAMTWVRQAPGKGLEWIGIIVASGGTYY |
| | | ASWAKGRFTISRTSTTVVMKITSPTTEDTATYFCARGGMGSDWGESLDAFDPWGQGTL |
| | | VTVSSASTKGPSVFPLAPCSRSTSESTAALGCLVKDYFPEPVTVSWNSGALTSGVHTF |
| | | PAVLQSSGLYSLSSVVTVPSSSLGTKTYTCNVDHKPSNTKVDKRVESKYGPPCPPCPA |
| | | PEFLGGPSVFLFPPKPKDTLMISRTPEVTCVVVDVSQEDPEVQFNWYVDGVEVHNAKT |
| | | KPREEQFNSTYRVVSVLTVLHQDWLNGKEYKCKVSNKGLPSSIEKTISKAKGQPREPQ |
| | | VYTLPPSQEEMTKNQVSLTCLVKGFYPSDIAVEWESNGQPENNYKTTPPVLDSDGSFF |
| | | LYSRLTVDKSRWQEGNVFSCSVMHEALHNHYTQKSLSLSLGK |
| 4 | B21A-HC1 | QSVEESGGRLVTPGTPLTLTCTVSGFSLNTYAMTWVRQAPGKGLEWIGIIVASGGTYY |
| | | ASWAKGRFTISRTSTTVVMKITSPTTEDTATYFCARGGMGSDWGESLDAFDPWGQGTL |
| | | VTVSSASTKGPSVFPLAPSSKSTSGGTAALGCLVKDYFPEPVTVSWNSGALTSGVHTF |
| | | PAVLQSSGLYSLSSVVTVPSSSLGTQTYICNVNHKPSNTKVDKRVEPKSCDKTHTCPP |
| | | CPAPELLGGPSVFLFPPKPKDTLMISRTPEVTCVVVDVSHEDPEVKENWYVDGVEVHN |
| | | AKTKPREEQYNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKTTPPVLDSDG |
| | | SFFLYSKLTVDKSRWQQGNVFSCSVMHEALHNHYTQKSLSLSPGK |
| 5 | B21A-LC | DIVMTQTPASVEAAVGGTVTINCQASGNIYSFLAWYQQKPGQPPKLLIYTASNLETGV |
| | | PSRFSGSGSGTEYTLTISGVQCDDAATYYCLGGLYSSDVSYAFGGGTEVVVKRIVAAP |
| | | SVFIFPPSDEQLKSGTASVVCLLNNFYPREAKVQWKVDNALQSGNSQESVTEQDSKDS |
| | | TYSLSSTLTLSKADYEKHKVYACEVTHQGLSSPVTKSFNRGEC |
| 6 | B21H-VH1 | QVQLVESGGGVVQPGTSLRLSCAASGFSLNTYAMTWVRQAPGKGLEWVAIIVASGGTY |
| | | YASWAKGRFTISRDTSKNTVVMQITSLRAEDTAVYFCARGGMGSDWGESLDAFDPWGQ |
| | | GTLVTVSS |
| 7 | B21H-VH2 | EVQLLESGGGLVQPGGSLRLSCAASGFSLNTYAMTWVRQAPGKGLEWVSIIVASGGTY |
| | | YASWAKGRFTISRDNSKNTLYLQMNSLTVEDTAVYFCARGGMGSDWGESLDAFDPWGQ |
| | | GTLVTVSS |
| 8 | B21H-VH3 | EVQLVESGGDLAQPGGSLRLSCAVSGFSLNTYAMTWVRQAPGKGLEWVSIIVASGGTY |
| | | YASWAKGRFTISRDNSKNTVYLQMTSLRAEDTALYFCARGGMGSDWGESLDAFDPWGQ |
| | | GTLVTVSS |
| 9 | B21H-VH4 | EVQLVESGGGLVQPGGSLRLSCAASGFSLNTYAMTWVRQAPGKGLEWISIIVASGGTY |
| | | YASWAKGRFTISRDNAKSSVYLQLTSLTTEDTAVYYCARGGMGSDWGESLDAFDPWGQ |
| | | GTMVTVSS |
| 10 | B21H-VH5 | QVQLEESGGRLVQPGTSLRLSCAVSGFSLNTYAMTWVRQAPGKGLEWIGIIVASGGTY |
| | | YASWAKGRFTISRDTSKNTLYLQITSLRAEDTATYFCARGGMGSDWGESLDAFDPWGQ |
| | | GTLVTVSS |
| 11 | B21H-VL1 | DIQMTQSPSTLSASVGDRVTITCQASGNIYSFLAWYQQKPGKAPKLLIYTASNLETGV |
| | | PSRFSGSGSGTEYTLTISSLQPDDFATYYCLGGLYSSDVSYAFGPGTKVEIK |
| 12 | B21H-VL2 | DIQMTQSPSSLSASVGDRVTITCQASGNIYSFLAWYQQKPGKVPKLLIYTASNLETGV |
| | | PSRFSGSGSGTDYTLTISSLQSEDAATYYCLGGLYSSDVSYAFGGGTKVVIK |
| 13 | B21H-VL3 | DIVMTQSPSSVSASVGDRVTITCQASGNIYSFLAWYQQKPGKAPKLLIYTASNLETGV |
| | | PSRFSGSGSGTDFTLTISSLQPEDFATYYCLGGLYSSDVSYAFGGGTKVVIK |
| 14 | B21H-VL4 | DVVMTQSPSTVSASVGDRVTLTCQASGNIYSFLAWYQQKPGQPPKLLIYTASNLETGV |
| | | PDRFSGSGSGTDYTLTISSLQADDFATYYCLGGLYSSDVSYAFGQGTKVEIK |
| 15 | B21H-VL5 | DIQMTQTPSTVEASVGGTVTITCQASGNIYSFLAWYQQKPGQPPKLLIYTASNLETGV |
| | | PSRFSGSGSGTEYTLTISGLQSDDAATYYCLGGLYSSDVSYAFGGGTEVVVK |
| 16 | B21A CDR- | GFSLNTYA |
| | H1 IMGT | |
| 17 | B21A CDR- | IVASGGT |
| | H2 IMGT | |
| 18 | B21A CDR- | ARGGMGSDWGESLDAFDP |
| | H3 IMGT | |
| 19 | B21A CDR- | GNIYSF |
| | L1 IMGT | |
| 20 | B21A CDR- | TAS |
| | L2 IMGT | |
| 21 | B21A CDR- | LGGLYSSDVSYA |
| | L3 IMGT | |
| 22 | B21A CDR- | TYAMT |
| | H1 Kabat | |
| 23 | B21A CDR- | IIVASGGTYYASWAKG |
| | H2 Kabat | |
| 24 | B21A CDR- | GGMGSDWGESLDAFDP |
| | H3 Kabat | |
| 25 | B21A CDR- | QASGNIYSFLA |
| | L1 Kabat | |
| 26 | B21A CDR- | TASNLET |
| | L2 Kabat | |
| 27 | B21A CDR- | LGGLYSSDVSYA |
| | L3 Kabat | |
| 28 | B21H-H1C4 | QVQLVESGGGVVQPGTSLRLSCAASGFSLNTYAMTWVRQAPGKGLEWVAIIVASGGTY |
| | | YASWAKGRFTISRDTSKNTVVMQITSLRAEDTAVYFCARGGMGSDWGESLDAFDPWGQ |
| | | GTLVTVSSASTKGPSVFPLAPCSRSTSESTAALGCLVKDYFPEPVTVSWNSGALTSGV |
| | | HTFPAVLQSSGLYSLSSVVTVPSSSLGTKTYTCNVDHKPSNTKVDKRVESKYGPPCPP |
| | | CPAPEFLGGPSVFLFPPKPKDTLMISRTPEVTCVVVDVSQEDPEVQFNWYVDGVEVHN |
| | | AKTKPREEQFNSTYRVVSVLTVLHQDWLNGKEYKCKVSNKGLPSSIEKTISKAKGQPR |
| | | EPQVYTLPPSQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK |
| 29 | B21H-H2C4 | EVQLLESGGGLVQPGGSLRLSCAASGFSLNTYAMTWVRQAPGKGLEWVSIIVASGGTY |
| | | YASWAKGRFTISRDNSKNTLYLQMNSLTVEDTAVYFCARGGMGSDWGESLDAFDPWGQ |
| | | GTLVTVSSASTKGPSVFPLAPCSRSTSESTAALGCLVKDYFPEPVTVSWNSGALTSGV |
| | | HTFPAVLQSSGLYSLSSVVTVPSSSLGTKTYTCNVDHKPSNTKVDKRVESKYGPPCPP |
| | | CPAPEFLGGPSVFLFPPKPKDTLMISRTPEVTCVVVDVSQEDPEVQFNWYVDGVEVHN |
| | | AKTKPREEQFNSTYRVVSVLTVLHQDWLNGKEYKCKVSNKGLPSSIEKTISKAKGQPR |
| | | EPQVYTLPPSQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK |
| 30 | B21H-H3C4 | EVQLVESGGDLAQPGGSLRLSCAVSGFSLNTYAMTWVRQAPGKGLEWVSIIVASGGTY |
| | | YASWAKGRFTISRDNSKNTVYLQMTSLRAEDTALYFCARGGMGSDWGESLDAFDPWGQ |
| | | GTLVTVSSASTKGPSVFPLAPCSRSTSESTAALGCLVKDYFPEPVTVSWNSGALTSGV |
| | | HTFPAVLQSSGLYSLSSVVTVPSSSLGTKTYTCNVDHKPSNTKVDKRVESKYGPPCPP |
| | | CPAPEFLGGPSVFLFPPKPKDTLMISRTPEVTCVVVDVSQEDPEVQFNWYVDGVEVHN |
| | | AKTKPREEQFNSTYRVVSVLTVLHQDWLNGKEYKCKVSNKGLPSSIEKTISKAKGQPR |
| | | EPQVYTLPPSQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK |
| 31 | B21H-H4C4 | EVQLVESGGGLVQPGGSLRLSCAASGFSLNTYAMTWVRQAPGKGLEWISIIVASGGTY |
| | | YASWAKGRFTISRDNAKSSVYLQLTSLTTEDTAVYYCARGGMGSDWGESLDAFDPWGQ |
| | | GTMVTVSSASTKGPSVFPLAPCSRSTSESTAALGCLVKDYFPEPVTVSWNSGALTSGV |
| | | HTFPAVLQSSGLYSLSSVVTVPSSSLGTKTYTCNVDHKPSNTKVDKRVESKYGPPCPP |
| | | CPAPEFLGGPSVFLFPPKPKDTLMISRTPEVTCVVVDVSQEDPEVQFNWYVDGVEVHN |
| | | AKTKPREEQFNSTYRVVSVLTVLHQDWLNGKEYKCKVSNKGLPSSIEKTISKAKGQPR |
| | | EPQVYTLPPSQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK |
| 32 | B21H-H5C4 | QVQLEESGGRLVQPGTSLRLSCAVSGFSLNTYAMTWVRQAPGKGLEWIGIIVASGGTY |
| | | YASWAKGRFTISRDTSKNTLYLQITSLRAEDTATYFCARGGMGSDWGESLDAFDPWGQ |
| | | GTLVTVSSASTKGPSVFPLAPCSRSTSESTAALGCLVKDYFPEPVTVSWNSGALTSGV |
| | | HTFPAVLQSSGLYSLSSVVTVPSSSLGTKTYTCNVDHKPSNTKVDKRVESKYGPPCPP |
| | | CPAPEFLGGPSVFLFPPKPKDTLMISRTPEVTCVVVDVSQEDPEVQFNWYVDGVEVHN |
| | | AKTKPREEQFNSTYRVVSVLTVLHQDWLNGKEYKCKVSNKGLPSSIEKTISKAKGQPR |
| | | EPQVYTLPPSQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK |
| 33 | B21H-H1C1 | QVQLVESGGGVVQPGTSLRLSCAASGFSLNTYAMTWVRQAPGKGLEWVAIIVASGGTY |
| | | YASWAKGRFTISRDTSKNTVVMQITSLRAEDTAVYFCARGGMGSDWGESLDAFDPWGQ |
| | | GTLVTVSSASTKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKRVEPKSCDKTHT |
| | | CPPCPAPELLGGPSVFLFPPKPKDTLMISRTPEVTCVVVDVSHEDPEVKENWYVDGVE |
| | | VHNAKTKPREEQYNSTYRVVSVLTVLHQDWLNGKEYKCKVSNKALPAPIEKTISKAKG |
| | | QPREPQVYTLPPSREEMTKNQVSLTCLVKGFYPSDIAVEWESNGQPENNYKTTPPVLD |
| | | SDGSFFLYSKLTVDKSRWQQGNVFSCSVMHEALHNHYTQKSLSLSPGK |
| 34 | B21H-H2C1 | EVQLLESGGGLVQPGGSLRLSCAASGFSLNTYAMTWVRQAPGKGLEWVSIIVASGGTY |
| | | YASWAKGRFTISRDNSKNTLYLQMNSLTVEDTAVYFCARGGMGSDWGESLDAFDPWGQ |
| | | GTLVTVSSASTKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKRVEPKSCDKTHT |
| | | CPPCPAPELLGGPSVFLFPPKPKDTLMISRTPEVTCVVVDVSHEDPEVKENWYVDGVE |
| | | VHNAKTKPREEQYNSTYRVVSVLTVLHQDWLNGKEYKCKVSNKALPAPIEKTISKAKG |
| | | QPREPQVYTLPPSREEMTKNQVSLTCLVKGFYPSDIAVEWESNGQPENNYKTTPPVLD |
| | | SDGSFFLYSKLTVDKSRWQQGNVFSCSVMHEALHNHYTQKSLSLSPGK |
| 35 | B21H-H3C1 | EVQLVESGGDLAQPGGSLRLSCAVSGFSLNTYAMTWVRQAPGKGLEWVSIIVASGGTY |
| | | YASWAKGRFTISRDNSKNTVYLQMTSLRAEDTALYFCARGGMGSDWGESLDAFDPWGQ |
| | | GTLVTVSSASTKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKRVEPKSCDKTHT |
| | | CPPCPAPELLGGPSVFLFPPKPKDTLMISRTPEVTCVVVDVSHEDPEVKENWYVDGVE |
| | | VHNAKTKPREEQYNSTYRVVSVLTVLHQDWLNGKEYKCKVSNKALPAPIEKTISKAKG |
| | | QPREPQVYTLPPSREEMTKNQVSLTCLVKGFYPSDIAVEWESNGQPENNYKTTPPVLD |
| | | SDGSFFLYSKLTVDKSRWQQGNVFSCSVMHEALHNHYTQKSLSLSPGK |
| 36 | B21H-H4C1 | EVQLVESGGGLVQPGGSLRLSCAASGFSLNTYAMTWVRQAPGKGLEWISIIVASGGTY |
| | | YASWAKGRFTISRDNAKSSVYLQLTSLTTEDTAVYYCARGGMGSDWGESLDAFDPWGQ |
| | | GTMVTVSSASTKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKRVEPKSCDKTHT |
| | | CPPCPAPELLGGPSVFLFPPKPKDTLMISRTPEVTCVVVDVSHEDPEVKENWYVDGVE |
| | | VHNAKTKPREEQYNSTYRVVSVLTVLHQDWLNGKEYKCKVSNKALPAPIEKTISKAKG |
| | | QPREPQVYTLPPSREEMTKNQVSLTCLVKGFYPSDIAVEWESNGQPENNYKTTPPVLD |
| | | SDGSFFLYSKLTVDKSRWQQGNVFSCSVMHEALHNHYTQKSLSLSPGK |
| 37 | B21H-H5C1 | QVQLEESGGRLVQPGTSLRLSCAVSGFSLNTYAMTWVRQAPGKGLEWIGIIVASGGTY |
| | | YASWAKGRFTISRDTSKNTLYLQITSLRAEDTATYFCARGGMGSDWGESLDAFDPWGQ |
| | | GTLVTVSSASTKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKRVEPKSCDKTHT |
| | | CPPCPAPELLGGPSVFLFPPKPKDTLMISRTPEVTCVVVDVSHEDPEVKENWYVDGVE |
| | | VHNAKTKPREEQYNSTYRVVSVLTVLHQDWLNGKEYKCKVSNKALPAPIEKTISKAKG |
| | | QPREPQVYTLPPSREEMTKNQVSLTCLVKGFYPSDIAVEWESNGQPENNYKTTPPVLD |
| | | SDGSFFLYSKLTVDKSRWQQGNVFSCSVMHEALHNHYTQKSLSLSPGK |
| 38 | B21H-L1 | DIQMTQSPSTLSASVGDRVTITCQASGNIYSFLAWYQQKPGKAPKLLIYTASNLETGV |
| | | PSRFSGSGSGTEYTLTISSLQPDDFATYYCLGGLYSSDVSYAFGPGTKVEIKRTVAAP |
| | | SVFIFPPSDEQLKSGTASVVCLLNNFYPREAKVQWKVDNALQSGNSQESVTEQDSKDS |
| | | TYSLSSTLTLSKADYEKHKVYACEVTHQGLSSPVTKSFNRGEC |
| 39 | B21H-L2 | DIQMTQSPSSLSASVGDRVTITCQASGNIYSFLAWYQQKPGKVPKLLIYTASNLETGV |
| | | PSRFSGSGSGTDYTLTISSLQSEDAATYYCLGGLYSSDVSYAFGGGTKVVIKRTVAAP |
| | | SVFIFPPSDEQLKSGTASVVCLLNNFYPREAKVQWKVDNALQSGNSQESVTEQDSKDS |
| | | TYSLSSTLTLSKADYEKHKVYACEVTHQGLSSPVTKSFNRGEC |
| 40 | B21H-L3 | DIVMTQSPSSVSASVGDRVTITCQASGNIYSFLAWYQQKPGKAPKLLIYTASNLETGV |
| | | PSRFSGSGSGTDFTLTISSLQPEDFATYYCLGGLYSSDVSYAFGGGTKVVIKRTVAAP |
| | | SVFIFPPSDEQLKSGTASVVCLLNNFYPREAKVQWKVDNALQSGNSQESVTEQDSKDS |
| | | TYSLSSTLTLSKADYEKHKVYACEVTHQGLSSPVTKSFNRGEC |
| 41 | B21H-L4 | DVVMTQSPSTVSASVGDRVTLTCQASGNIYSFLAWYQQKPGQPPKLLIYTASNLETGV |
| | | PDRFSGSGSGTDYTLTISSLQADDFATYYCLGGLYSSDVSYAFGQGTKVEIKRTVAAP |
| | | SVFIFPPSDEQLKSGTASVVCLLNNFYPREAKVQWKVDNALQSGNSQESVTEQDSKDS |
| | | TYSLSSTLTLSKADYEKHKVYACEVTHQGLSSPVTKSFNRGEC |
| 42 | B21H-L5 | DIQMTQTPSTVEASVGGTVTITCQASGNIYSFLAWYQQKPGQPPKLLIYTASNLETGV |
| | | PSRFSGSGSGTEYTLTISGLQSDDAATYYCLGGLYSSDVSYAFGGGTEVVVKRTVAAP |
| | | SVFIFPPSDEQLKSGTASVVCLLNNFYPREAKVQWKVDNALQSGNSQESVTEQDSKDS |
| | | TYSLSSTLTLSKADYEKHKVYACEVTHQGLSSPVTKSFNRGEC |
| 43 | COMP1 VH | QVQLKESGPGLVAPSQSLSITCTVSGFSLTNYGVSWVRQPPGKGLEWLGIIWGDGSTN |
| | | YHSALISRLSISKDNSKSQVFLKLNSLQADDTATYYCAKPNWDTYAMDFWGQGTSVTV |
| | | SSASTKGPSVFPLAPSSKSTSGGTAALGCLVKDYFPEPVTVSWNSGALTSGVHTFPAV |
| | | LQSSGLYSLSSVVTVPSSSLGTQTYICNVNHKPSNTKVDKRVEPKSCDKTHTCPPCPA |
| | | PELLGGPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQ |
| | | VYTLPPSREEMTKNQVSLTCLVKGFYPSDIAVEWESNGQPENNYKTTPPVLDSDGSFF |
| | | LYSKLTVDKSRWQQGNVFSCSVMHEALHNHYTQKSLSLSPGK |
| 44 | COMP1 VL | DIQMTQTTSSLSASLGDRVTISCRASQDISNFLNWYQQKPDGTVKLLIYCTSKLHSGV |
| | | PSRFSGSGSETDYSLTISNLEQEDIATYFCQQGNTLPPTFGGGTKLEIIRTVAAPSVE |
| | | IFPPSDEQLKSGTASVVCLLNNFYPREAKVQWKVDNALQSGNSQESVTEQDSKDSTYS |
| | | LSSTLTLSKADYEKHKVYACEVTHQGLSSPVTKSFNRGEC |
| 45 | COMP2 VH | QIQLVQSGPELKKPGETVKISCKASGYTFTNYGMNWVKQAPGKGLKWMGWINTYIGEP |
| | | IYADDFKGRFAFSLETSASTAYLQINNLKNEDMATYFCARRSDYDGYAMDYWGQGTSV |
| | | TVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPC |
| | | PAPELLGGPSVFLFPPKPKDTLMISRTPEVTCVVVDVSHEDPEVKENWYVDGVEVHNA |
| | | KTKPREEQYNSTYRVVSVLTVLHQDWLNGKEYKCKVSNKALPAPIEKTISKAKGQPRE |
| | | PQVYTLPPSREEMTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| 46 | COMP2 VL | DIVMSQSPSSLAVSVGERVTMSCKSSQSLLYSGNQKNYLAWYQQKPGQSPKLLIYWAS |
| | | TRESGVPDRFTGSGSGTDFTLTISSVKAEDLAVYYCQQHDSYPTFGGGSRLEIKRTVA |
| | | APSVFIFPPSDEQLKSGTASVVCLLNNFYPREAKVQWKVDNALQSGNSQESVTEQDSK |
| | | DSTYSLSSTLTLSKADYEKHKVYACEVTHQGLSSPVTKSFNRGEC |
| 47 | COMP3 VH | QVQLVQSGAEVKKPGASVKVSCKASGYTFTDYYINWVRQAPGQGLEWMGNVNPNDGGT |
| | | TYNQKFKGRVTMTTDTSTSTAYMELRSLRSDDTAVYYCARREIYFYGTIYYYAMDYWG |
| | | QGTLVTVSSASTKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDKKVEPKSCDKTH |
| | | TCPPCPAPELLGGPSVFLFPPKPKDTLMISRTPEVTCVVVDVSHEDPEVKENWYVDGV |
| | | EVHNAKTKPREEQYGSTYRVVSVLTVLHQDWLNGKEYKCKVSNKALPAPIEKTISKAK |
| | | GQPREPQVYTLPPSREEMTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| 48 | COMP3 VL | DIQLTQSPSFLSASVGDRVTITCRASESVDYYGNSFMYWYQQKPGKAPKLLIYFASNL |
| | | ESGVPSRFSGSGSGTEFTLTISSLQPEDFATYYCQQNNEDPWTFGGGTKVEIKRTVAA |
| | | PSVFIFPPSDEQLKSGTASVVCLLNNFYPREAKVQWKVDNALQSGNSQESVTEQDSKD |
| | | STYSLSSTLTLSKADYEKHKVYACEVTHQGLSSPVTKSFNRGEC |
| 49 | COMP4 VH | EVQLQQWGAGLLKPSETLSLTCAVYGGSFSGYYWSWIRQPPGKGLEWIGEINHAGSTN |
| | | YNPSLKSRVTISVDTSKNQFSLKLSSVTAADTAVYYCARLPTRWVTTRYFDLWGRGTL |
| | | VTVSSASTKGPSVFPLAPCSRSTSESTAALGCLVKDYFPEPVTVSWNSGALTSGVHTF |
| | | PAVLQSSGLYSLSSVVTVPSSSLGTKTYTCNVDHKPSNTKVDKRVESKYGPPCPPCPA |
| | | PEFLGGPSVFLFPPKPKDTLMISRTPEVTCVVVDVSQEDPEVQFNWYVDGVEVHNAKT |
| | | KPREEQFNSTYRVVSVLTVLHQDWLNGKEYKCKVSNKGLPSSIEKTISKAKGQPREPQ |
| | | VYTLPPSQEEMTKNQVSLTCLVKGFYPSDIAVEWESNGQPENNYKTTPPVLDSDGSFF |
| | | LYSRLTVDKSRWQEGNVFSCSVMHEALHNHYTQKSLSLSLGK |
| 50 | COMP4 VL | ESVLTQPPSVSGAPGQRVTISCTGSSSNIGAGYDVHWYQQLPGTAPKLLIYGDSNRPS |
| | | GVPDRFSVSKSGASASLAITGLQAEDEADYYCQSFDNSLSAYVFGGGTQLTVLGQPKA |
| | | APSVTLFPPSSEELQANKATLVCLISDFYPGAVTVAWKADSSPVKAGVETTTPSKQSN |
| | | NKYAASSYLSLTPEQWKSHRSYSCQVTHEGSTVEKTVAPTECS |

1. An antibody product comprising a variable region that specifically binds human LILRB2 and LILRB1, the variable region comprising:
a) a first domain comprising a CDR-H1 set forth in SEQ ID NO: 16, a CDR-H2 set forth in SEQ ID NO: 17, and a CDR-H3 set forth in SEQ ID NO: 18, and a second domain comprising a CDR-L1 set forth in SEQ ID NO: 19, a CDR-L2 with the sequence TAS, and a CDR-L3 set forth in SEQ ID NO: 21, as specified using the IMGT CDR definition; or
b) a first domain comprising a CDR-H1 set forth in SEQ ID NO: 22, a CDR-H2 set forth in SEQ NO: 23, and a CDR-H3 set forth in SEQ ID NO: 24, and a second domain comprising a CDR-L1 set forth in SEQ ID NO: 25, a CDR-L2 set forth in SEQ ID NO: 26, and a CDR-L3 set forth in SEQ ID NO: 27, as specified using the Kabat CDR definition.

2. The antibody product of claim 1, in which the variable region comprises a heavy chain variable domain comprising:
a) an amino acid sequence at least 80% identical to SEQ ID NO: 1, 6, 7, 8, 9 or 10; or
b) an amino acid sequence set forth in SEQ ID NO: 1, 6, 7, 8, 9 or 10.

3. The antibody product of claim 1, in which the variable region comprises a light chain variable domain comprising:
a) an amino acid sequence at least 80% identical to SEQ ID NO: 2, 11, 12, 13, 14 or 15; or
b) an amino acid sequence set forth in SEQ ID NO: 2, 11, 12, 13, 14 or 15.

4. The antibody product of claim 1, in which the variable region comprises:
a) a heavy chain variable domain comprising SEQ ID NO: 1; and a light chain variable domain SEQ ID NO: 2;
b) a heavy chain variable domain comprising SEQ ID NO: 6; and a light chain variable domain comprising SEQ ID NO: 11;
c) a heavy chain variable domain comprising SEQ ID NO: 6; and a light chain variable domain comprising SEQ ID NO: 12;
d) a heavy chain variable domain comprising SEQ ID NO: 6; and a light chain variable domain comprising SEQ ID NO: 13;
e) a heavy chain variable domain comprising SEQ ID NO: 6; and a light chain variable domain comprising SEQ ID NO: 14;
f) a heavy chain variable domain comprising SEQ ID NO: 6; and a light chain variable domain comprising SEQ ID NO: 15;
g) a heavy chain variable domain comprising SEQ ID NO: 7; and a light chain variable comprising SEQ ID NO: 11;
h) a heavy chain variable domain comprising SEQ ID NO: 7; and a light chain variable domain comprising SEQ ID NO: 12;
i) a heavy chain variable domain comprising SEQ ID NO: 7; and a light chain variable domain comprising SEQ ID NO: 13;
j) a heavy chain variable domain comprising SEQ ID NO: 7; a light chain variable domain comprising SEQ ID NO: 14;
k) a heavy chain variable domain comprising SEQ ID NO: 7; and a light chain variable domain comprising SEQ ID NO: 15;
l) a heavy chain variable domain comprising SEQ ID NO: 8; and a light chain variable domain comprising SEQ ID NO: 11;
m) a heavy chain variable domain comprising SEQ ID NO: 8; and a light chain variable domain comprising SEQ ID NO: 12;
n) a heavy chain variable domain comprising SEQ ID NO: 8; and a light chain variable domain comprising SEQ ID NO: 13;
o) a heavy chain variable domain comprising SEQ ID NO: 8; and a light chain variable domain comprising SEQ ID NO: 14;
p) a heavy chain variable domain comprising SEQ ID NO: 8; and a light chain variable domain comprising SEQ ID NO: 15;
q) a heavy chain variable domain comprising SEQ ID NO: 9; a light chain variable domain comprising SEQ ID NO: 11;
r) a heavy chain variable domain comprising SEQ ID NO: 9; and a light chain variable domain comprising SEQ ID NO: 12;
s) a heavy chain variable domain comprising SEQ ID NO: 9; and a light chain variable domain comprising SEQ ID NO: 13;
t) a heavy chain variable domain comprising SEQ ID NO: 9; and a light chain variable domain comprising SEQ ID NO: 14;
u) a heavy chain variable domain comprising SEQ ID NO: 9; and a light chain variable domain comprising SEQ ID NO: 15;
v) a heavy chain variable domain comprising SEQ ID NO: 10; and a light chain variable domain comprising SEQ ID NO: 11;
w) a heavy chain variable domain comprising SEQ ID NO: 10; and a light chain variable domain comprising SEQ ID NO: 12;
x) a heavy chain variable domain comprising SEQ ID NO: 10; and a light chain variable domain comprising SEQ ID NO: 13;
y) a heavy chain variable domain comprising SEQ ID NO: 10; and a light chain variable domain comprising SEQ ID NO: 14; or
z) a heavy chain variable domain comprising SEQ ID NO: 10; and a light chain variable domain comprising SEQ ID NO: 15.

5. (canceled)

6. (canceled)

7. (canceled)

8. (canceled)

9. (canceled)

10. (canceled)

11. The antibody product of claim 10, in which the heavy chain amino acid sequence comprises:
a) an amino acid sequence at least 80% identical to SEQ ID NO: 3, SEQ ID NO: 28, SEQ ID NO: 29, SEQ ID NO: 30, SEQ ID NO: 31, or SEQ ID NO: 32; or
b) an amino acid sequence set forth in SEQ ID NO: 3, SEQ ID NO: 28, SEQ ID NO: 29, SEQ ID NO: 30, SEQ ID NO: 31, or SEQ ID NO: 32.

12. The antibody product of claim 9, in which the heavy chain constant domain is an IgG4 constant domain.

13. The antibody product of 12, in which the heavy chain amino acid sequence comprises:
a) an amino acid sequence at least 80% identical to SEQ ID NO: 4, SEQ ID NO: 33, SEQ ID NO: 34, SEQ ID NO: 35, SEQ ID NO: 36, or SEQ ID NO: 37; or
b) an amino acid sequence set forth in SEQ ID NO: 4, SEQ ID NO: 33, SEQ ID NO: 34, SEQ ID NO: 35, SEQ ID NO: 36, or 37.

14. (canceled)

15. (canceled)

16. The antibody product of 12, in which the light chain amino acid sequence comprises:
a) an amino acid sequence at least 80% identical to SEQ ID NO: 5, SEQ ID NO: 38, SEQ ID NO: 39, SEQ ID NO: 40, SEQ ID NO: 41, or SEQ ID NO: 42; or
b) an amino acid sequence set forth in SEQ ID NO: 5, SEQ ID NO: 38, SEQ ID NO: 39, SEQ ID NO: 40, SEQ ID NO: 41, or SEQ ID NO: 42.

17. An antibody product that binds human LILRB2, comprising:
a) an IgG1 heavy chain having an amino acid sequence comprising SEQ ID NO: 1; and a kappa light chain having an amino acid sequence comprising SEQ ID NO: 2;
b) an IgG1 heavy chain having an amino acid sequence comprising SEQ ID NO: 6; and a kappa light chain having an amino acid sequence comprising SEQ ID NO: 11;
c) an IgG1 heavy chain having an amino acid sequence comprising SEQ ID NO: 6; and a kappa light chain having an amino acid sequence comprising SEQ ID NO: 12;
d) an IgG1 heavy chain having an amino acid sequence comprising SEQ ID NO: 6; and a kappa light chain having an amino acid sequence comprising SEQ ID NO: 13;
e) an IgG1 heavy chain having an amino acid sequence comprising SEQ ID NO: 6; and a kappa light chain having an amino acid sequence comprising SEQ ID NO: 14;
f) an IgG1 heavy chain having an amino acid sequence comprising SEQ ID NO: 6; and a kappa light chain having an amino acid sequence comprising SEQ ID NO: 15;
g) an IgG1 heavy chain having an amino acid sequence comprising SEQ ID NO: 7; and a kappa light chain having an amino acid sequence comprising SEQ ID NO: 11;
h) an IgG1 heavy chain having an amino acid sequence comprising SEQ ID NO: 7; and a kappa light chain having an amino acid sequence comprising SEQ ID NO: 12;
i) an IgG1 heavy chain having an amino acid sequence comprising SEQ ID NO: 7; and a kappa light chain having an amino acid sequence comprising SEQ ID NO: 13;
j) an IgG1 heavy chain having an amino acid sequence comprising SEQ ID NO: 7; and a kappa light chain having an amino acid sequence comprising SEQ ID NO: 14;
k) an IgG1 heavy chain having an amino acid sequence comprising SEQ ID NO: 7; and a kappa light chain having an amino acid sequence comprising SEQ ID NO: 15;
l) an IgG1 heavy chain having an amino acid sequence comprising SEQ ID NO: 8; and a kappa light chain having an amino acid sequence comprising SEQ ID NO: 11;
m) an IgG1 heavy chain having an amino acid sequence comprising SEQ ID NO: 8; and a kappa light chain having an amino acid sequence comprising SEQ ID NO: 12;
n) an IgG1 heavy chain having an amino acid sequence comprising SEQ ID NO: 8; and a kappa light chain having an amino acid sequence comprising SEQ ID NO: 13;
o) an IgG1 heavy chain having an amino acid sequence comprising SEQ ID NO: 8; and a kappa light chain having an amino acid sequence comprising SEQ ID NO: 14;
p) an IgG1 heavy chain having an amino acid sequence comprising SEQ ID NO: 8; and a kappa light chain having an amino acid sequence comprising SEQ ID NO: 15;
q) an IgG1 heavy chain having an amino acid sequence comprising SEQ ID NO: 9; and a kappa light chain having an amino acid sequence comprising SEQ ID NO: 11;
r) an IgG1 heavy chain having an amino acid sequence comprising SEQ ID NO: 9; and a kappa light chain having an amino acid sequence comprising SEQ ID NO: 12;
s) an IgG1 heavy chain having an amino acid sequence comprising SEQ ID NO: 9; and a kappa light chain having an amino acid sequence comprising SEQ ID NO: 13;
t) an IgG1 heavy chain having an amino acid sequence comprising SEQ ID NO: 9; and a kappa light chain having an amino acid sequence comprising SEQ ID NO: 14;
u) an IgG1 heavy chain having an amino acid sequence comprising SEQ ID NO: 9; and a kappa light chain having an amino acid sequence comprising SEQ ID NO: 15;
v) an IgG1 heavy chain having an amino acid sequence comprising SEQ ID NO: 10; and a kappa light chain having an amino acid sequence comprising SEQ ID NO: 11;
w) an IgG1 heavy chain having an amino acid sequence comprising SEQ ID NO: 10; and a kappa light chain having an amino acid sequence comprising SEQ ID NO: 12;
x) an IgG1 heavy chain having an amino acid sequence comprising SEQ ID NO: 10; and a kappa light chain having an amino acid sequence comprising SEQ ID NO: 13;
y) an IgG1 heavy chain having an amino acid sequence comprising SEQ ID NO: 10; and a kappa light chain having an amino acid sequence comprising SEQ ID NO: 14; or
z) an IgG1 heavy chain having an amino acid sequence comprising SEQ ID NO: 10; and a kappa light chain having an amino acid sequence comprising SEQ ID NO: 15.

18. An antibody product that binds human LILRB2, comprising:
a) an IgG4 heavy chain having an amino acid sequence comprising SEQ ID NO: 1; and a kappa light chain having an amino acid sequence comprising SEQ ID NO: 2;
b) an IgG4 heavy chain having an amino acid sequence comprising SEQ ID NO: 6; and a kappa light chain having an amino acid sequence comprising SEQ ID NO: 11;
c) an IgG4 heavy chain having an amino acid sequence comprising SEQ ID NO: 6; and a kappa light chain having an amino acid sequence comprising SEQ ID NO: 12;
d) an IgG4 heavy chain having an amino acid sequence comprising SEQ ID NO: 6; and a kappa light chain having an amino acid sequence comprising SEQ ID NO: 13;
e) an IgG4 heavy chain having an amino acid sequence comprising SEQ ID NO: 6; and a kappa light chain having an amino acid sequence comprising SEQ ID NO: 14;
f) an IgG4 heavy chain having an amino acid sequence comprising SEQ ID NO: 6; and a kappa light chain having an amino acid sequence comprising SEQ ID NO: 15;
g) an IgG4 heavy chain having an amino acid sequence comprising SEQ ID NO: 7; and a kappa light chain having an amino acid sequence comprising SEQ ID NO: 11;
h) an IgG4 heavy chain having an amino acid sequence comprising SEQ ID NO: 7; and a kappa light chain having an amino acid sequence comprising SEQ ID NO: 12;
i) an IgG4 heavy chain having an amino acid sequence comprising SEQ ID NO: 7; and a kappa light chain having an amino acid sequence comprising SEQ ID NO: 13;
j) an IgG4 heavy chain having an amino acid sequence comprising SEQ ID NO: 7; and a kappa light chain having an amino acid sequence comprising SEQ ID NO: 14;
k) an IgG4 heavy chain having an amino acid sequence comprising SEQ ID NO: 7; and a kappa light chain having an amino acid sequence comprising SEQ ID NO: 15;
l) an IgG4 heavy chain having an amino acid sequence comprising SEQ ID NO: 8; and a kappa light chain having an amino acid sequence comprising SEQ ID NO: 11;
m) an IgG4 heavy chain having an amino acid sequence comprising SEQ ID NO: 8; and a kappa light chain having an amino acid sequence comprising SEQ ID NO: 12;
n) an IgG4 heavy chain having an amino acid sequence comprising SEQ ID NO: 8; and a kappa light chain having an amino acid sequence comprising SEQ ID NO: 13; or
o) an IgG4 heavy chain having an amino acid sequence comprising SEQ ID NO: 8; and a kappa light chain having an amino acid sequence comprising SEQ ID NO: 14.

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. A method of providing a cancer immunotherapy to a subject in need thereof, in which the cancer is associated with a presence of immunosuppressive macrophages, the method comprising administering to the subject a therapeutically effective amount of a pharmaceutical composition comprising an antibody product of claim 1.

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. An isolated nucleic acid, comprising a nucleotide sequence encoding a heavy chain variable domain of claim 2.

74. An isolated nucleic acid, comprising a nucleotide sequence encoding a light chain variable domain of claim 3.

75. (canceled)

76. (canceled)

77. An expression vector comprising the nucleic acids of claims 73 and 74.

78. A host cell comprising the expression vector of claim 77.

79. (canceled)

80. A method of producing an antibody product that binds human LILRB2, comprising:
a) growing a host cell comprising the expression vector of claim 77 under conditions such that the host cell expresses a protein comprising the immunoglobulin heavy chain variable domain and the immunoglobulin light chain variable domain, thereby producing the antibody product; and
b) purifying the antibody product.

81. A composition, comprising the antibody product of claim 1 and an excipient.

82. An article of manufacture, comprising the composition of claim 81 and a container.

83. (canceled)

84. A pharmaceutical composition, comprising an antibody product of claim 1 and a pharmaceutically acceptable excipient.

85. (canceled)

86. (canceled)

87. A method of detecting LILRB2 in a sample, tissue, or cell using the antibody product according to claim 1, comprising contacting the sample, tissue or cell with the antibody product and detecting the antibody product.

88. A method of reducing a biological activity of LILRB1 or LILRB2 in a subject in need thereof, said method comprising administering a therapeutically effective amount of the antibody product of claim 1.

89. (canceled)

90. A method of promoting an immune response in a subject in need thereof, the method comprising administering a therapeutically effective amount of the antibody product of claim 1.

91. A method of providing a cancer immunotherapy to a subject in need thereof, in which cells of the cancer express LILRB2, the method comprising administering to the subject a therapeutically effective amount of the antibody product of claim 1.

92. (canceled)

93. (canceled)

94. The method of claim 91, further comprising administering to the subject a PD-1 antagonist or a PD-L1 antagonist in an amount sufficient to relieve PD-1/PD-L1 axis-mediated immunosuppression of T cells in the subject, or a CTLA-4 antagonist in an amount sufficient to relieve CTLA-4 axis-mediated immunosuppression of T cells in the subject.

95. (canceled)

96. (canceled)

97. (canceled)

98. The antibody product of claim 1, in which the antibody product is detectably labeled or comprises a conjugated toxin, drug, receptor, enzyme, receptor ligand.

99. The antibody product claim 98, in which the antibody product comprises a therapeutic or cytotoxic moiety.

100. (canceled)

101. (canceled)

102. (canceled)

103. (canceled)

104. (canceled)

105. (canceled)

106. (canceled)

107. (canceled)

108. (canceled)

109. (canceled)

110. The antibody product of claim 1, comprising:
a) a heavy chain comprising SEQ ID NO: 1; and a light chain comprising SEQ ID NO: 2;
b) a heavy chain comprising SEQ ID NO: 6; and a light chain comprising SEQ ID NO: 11;
c) a heavy chain comprising SEQ ID NO: 6; and a light chain comprising SEQ ID NO: 12;
d) a heavy chain comprising SEQ ID NO: 6; and a light chain comprising SEQ ID NO: 13;
e) a heavy chain comprising SEQ ID NO: 6; and a light chain comprising SEQ ID NO: 14;
f) a heavy chain comprising SEQ ID NO: 6; and a light chain comprising SEQ ID NO: 15;
g) a heavy chain comprising SEQ ID NO: 7; and a light chain variable comprising SEQ ID NO: 11;
h) a heavy chain comprising SEQ ID NO: 7; and a light chain comprising SEQ ID NO: 12;
i) a heavy chain comprising SEQ ID NO: 7; and a light chain comprising SEQ ID NO: 13;
j) a heavy chain comprising SEQ ID NO: 7; a light chain comprising SEQ ID NO: 14;
k) a heavy chain comprising SEQ ID NO: 7; and a light chain comprising SEQ ID NO: 15;
l) a heavy chain comprising SEQ ID NO: 8; and a light chain comprising SEQ ID NO: 11;
m) a heavy chain comprising SEQ ID NO: 8; and a light chain comprising SEQ ID NO: 12;
n) a heavy chain comprising SEQ ID NO: 8; and a light chain comprising SEQ ID NO: 13;
o) a heavy chain comprising SEQ ID NO: 8; and a light chain comprising SEQ ID NO: 14;
p) a heavy chain comprising SEQ ID NO: 8; and a light chain comprising SEQ ID NO: 15;
q) a heavy chain comprising SEQ ID NO: 9; a light chain comprising SEQ ID NO: 11;
r) a heavy chain comprising SEQ ID NO: 9; and a light chain comprising SEQ ID NO: 12;
s) a heavy chain comprising SEQ ID NO: 9; and a light chain comprising SEQ ID NO: 13;
t) a heavy chain comprising SEQ ID NO: 9; and a light chain comprising SEQ ID NO: 14;
u) a heavy chain comprising SEQ ID NO: 9; and a light chain comprising SEQ ID NO: 15;
v) a heavy chain comprising SEQ ID NO: 10; and a light chain comprising SEQ ID NO: 11;
w) a heavy chain comprising SEQ ID NO: 10; and a light chain comprising SEQ ID NO: 12;
x) a heavy chain comprising SEQ ID NO: 10; and a light chain comprising SEQ ID NO: 13;
y) a heavy chain comprising SEQ ID NO: 10; and a light chain comprising SEQ ID NO: 14; or
z) a heavy chain comprising SEQ ID NO: 10; and a light chain comprising SEQ ID NO: 15.

111. The antibody product of claim 1, comprising:
a) a heavy chain comprising SEQ ID NO: 1; and a light chain comprising SEQ ID NO: 2;
b) a heavy chain comprising SEQ ID NO: 28 or SEQ ID NO: 33; and a light chain comprising SEQ ID NO: 38;
c) a heavy chain comprising SEQ ID NO: 28 or SEQ ID NO: 33; and a light chain comprising SEQ ID NO: 39;
d) a heavy chain comprising SEQ ID NO: 28 or SEQ ID NO: 33; and a light chain comprising SEQ ID NO: 40;
e) a heavy chain comprising SEQ ID NO: 28 or SEQ ID NO: 33; and a light chain comprising SEQ ID NO: 41;
f) a heavy chain comprising SEQ ID NO: 28 or SEQ ID NO: 33; and a light chain comprising SEQ ID NO: 42;
g) a heavy chain comprising SEQ ID NO: 29 or SEQ ID NO: 34; and a light chain comprising SEQ ID NO: 38;
h) a heavy chain comprising SEQ ID NO: 29 or SEQ ID NO: 34; and a light chain comprising SEQ ID NO: 39;
i) a heavy chain comprising SEQ ID NO: 29 or SEQ ID NO: 34; and a light chain comprising SEQ ID NO: 40;
j) a heavy chain comprising SEQ ID NO: 29 or SEQ ID NO: 34; and a light chain comprising SEQ ID NO: 41;
k) a heavy chain comprising SEQ ID NO: 29 or SEQ ID NO: 34; and a light chain comprising SEQ ID NO: 42;
l) a heavy chain comprising SEQ ID NO: 30 or SEQ ID NO: 35; and a light chain comprising SEQ ID NO: 38;
m) a heavy chain comprising SEQ ID NO: 30 or SEQ ID NO: 35; and a light chain comprising SEQ ID NO: 39;
n) a heavy chain comprising SEQ ID NO: 30 or SEQ ID NO: 35; and a light chain comprising SEQ ID NO: 40;
o) a heavy chain comprising SEQ ID NO: 30 or SEQ ID NO: 35; and a light chain comprising SEQ ID NO: 41;
p) a heavy chain comprising SEQ ID NO: 30 or SEQ ID NO: 35; and a light chain comprising SEQ ID NO: 42;
q) a heavy chain comprising SEQ ID NO: 31 or SEQ ID NO: 36; and a light chain comprising SEQ ID NO: 38;
r) a heavy chain comprising SEQ ID NO: 31 or SEQ ID NO: 36; and a light chain comprising SEQ ID NO: 39;
s) a heavy chain comprising SEQ ID NO: 31 or SEQ ID NO: 36; and a light chain comprising SEQ ID NO: 40;
t) a heavy chain comprising SEQ ID NO: 31 or SEQ ID NO: 36; and a light chain comprising SEQ ID NO: 41;
u) a heavy chain comprising SEQ ID NO: 31 or SEQ ID NO: 36; and a light chain comprising SEQ ID NO: 42;
v) a heavy chain comprising SEQ ID NO: 32 or SEQ ID NO: 37; and a light chain comprising SEQ ID NO: 38;
w) a heavy chain comprising SEQ ID NO: 32 or SEQ ID NO: 37; and a light chain comprising SEQ ID NO: 39;
x) a heavy chain comprising SEQ ID NO: 32 or SEQ ID NO: 37; and a light chain comprising SEQ ID NO: 40;
y) a heavy chain comprising SEQ ID NO: 32 or SEQ ID NO: 37; and a light chain comprising SEQ ID NO: 41; or
a heavy chain comprising SEQ ID NO: 32 or SEQ ID NO: 37; and a light chain comprising SEQ ID NO: 42.
